(19) United States
(12) Patent Application Publication
(10) Pub. No.: US 2009/0275727 A1
(43) Pub. Date: Nov. 5, 2009

(54) Peptide turn mimetics

(75) Inventors: Peter J. Cassidy, Ashgrove (AU); Paul Francis Alewood, Pullenvale (AU); Andrea Joy Vernall, Taringa (AU)

Correspondence Address: SCHWEGMAN, LUNDBERG & WOESSNER, P.A., P.O. BOX 2938, MINNEAPOLIS, MN, 55402, US

(73) Assignee: MIMETICA PTY LIMITED, Brisbane (AU)

(21) Appl. No.: 12/386,988

(22) Filed: Apr. 24, 2009

Related U.S. Application Data
(63) Continuation-in-part of application No. 09/647,054, filed on Feb. 6, 2001.

(30) Foreign Application Priority Data
Mar. 24, 1998 (AU) AUPP2548

(51) Int. Cl. C07K 5/12; C07K 7/08; C07K 5/083; C07D 243/08; C07D 487/04; C07D 233/02

(52) U.S. Cl. 530/317; 530/327; 530/331; 540/492; 540/502; 548/300.1

(57) ABSTRACT

This invention provides novel compounds useful for the synthesis of peptide mimetics, and describes the use of these compounds for the synthesis of novel reverse turn mimetics. The reverse turn mimetics of the invention have the general structure X wherein the variables are as defined herein.

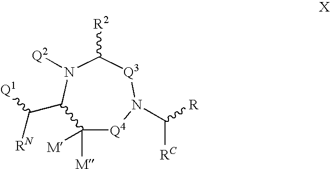

### CROSS REFERENCE TO RELATED APPLICATIONS

This application is a continuation in part of U.S. Ser. No. 09/647,054, a U.S. National Stage entry on Feb. 6, 2001, of PCT/AU99/00207, filed Mar. 24, 1999, published at WO1999/048913 on Sep. 30, 1999, which claims the priority of AU PP2548, filed Mar. 24, 1998, the priorities of which are claimed, and the disclosures of which are incorporated by reference herein in their entireties.

### FIELD OF THE INVENTION

THIS INVENTION relates to new compounds designed to be peptide turn mimetics, and to new compounds useful for the synthesis of peptide mimetics, especially turn mimetics. Peptide mimetics are used to reproduce the important structural and functional elements contained in a bio-active peptide sequence principally in order to develop novel pharmaceuticals with increased binding affinity, selectivity, stability and/or oral bioavailability compared to the bio-active peptide.

### BACKGROUND OF THE INVENTION

Reverse turns (beta and gamma turns and beta bulges) are localised on the protein surface (Kuntz 1972) and are of importance in protein interactions (Rose, Gierasch et al. 1985; Chalmers and Marshall 1995) (and references contained therein). In addition reverse turns are important structures of peptide hormones and other biologically active peptides and cyclic peptides (Giannis and Kolter 1993; Olson, Bolin et al. 1993; Kessler, Diefenbach et al. 1995).

Peptide mimetics and peptide turn mimetics have as their object the replacement of a peptide sequence (a peptide turn) with a new compound which retains the elements essential for biological activity, thereby enabling or facilitating the development of novel pharmaceuticals devoid of the inherent problems of peptides—namely flexibility and poor pharmacodynamics. The essential elements for biological activity are thought to be the peptide sidechain groups (Farmer and Ariëns 1982; Ball and Alewood 1990), therefore a peptide mimetic should include the side chain groups to have the best chance of retaining biological activity. A peptide mimetic may then take the form of a framework for displaying sidechain groups in an appropriate arrangement.

The majority of reverse turns are beta turns. The generally accepted definition of the beta turn is a sequence of four residues where the distance between the alpha carbons of residue (i) and residue (i+3) (defined as d) is less than 7 Å, and the central residues (i+1, i+2) are non-helical (Lewis, Momany et al. 1973). The general structure is shown below and includes the phi (φ) and psi (ψ) backbone dihedral angles that are used to describe the conformation of the peptide backbone. A schematic conversion of the beta turn to a beta turn mimetic is also shown—the peptide backbone is here replaced by an undefined framework.

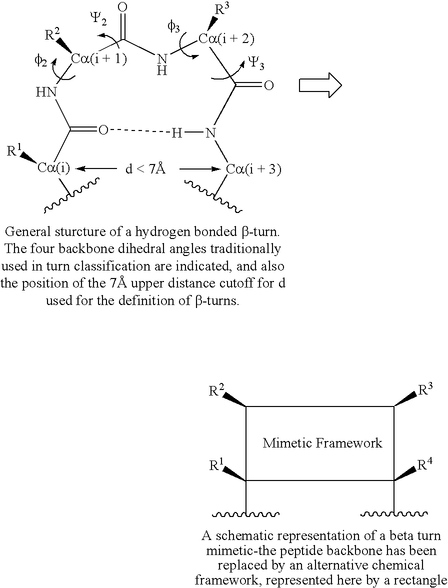

The gamma turn is generally defined by the presence of a hydrogen bond between C═O (i) and N—H (i+2) to form a pseudo seven membered ring as illustrated below (Milner-White 1988). Where the equivalent hydrogen bond is present in a beta turn a pseudo ten membered ring is formed.

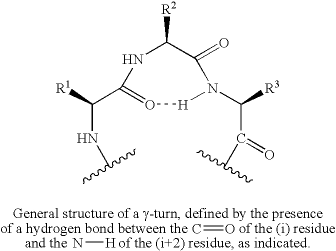

The chemical synthesis of a framework having four independent chiral groups each with a wide range of possible functionality (for example a beta turn mimetic) is a very significant synthetic challenge (Nakanishi and Kahn 1996) as illustrated by the fact that most proposed beta turn mimetics either do not provide for the incorporation of any sidechain functionality, or provide for a limited range of functionality, and at a limited number of positions. Reference may be made to reviews by Ball and by Hölzemann for illustration of these points (Ball and Alewood 1990; Hölzemann 1991; Hölzemann 1991). In the case of mimetics that do provide for the incorporation of sidechain functionality, the syntheses are often complex and lengthy, and most seriously may require a different synthetic method for different sidechain sequences (i.e. the synthetic method is not generic). For example in the work of Callahan, Huffman and Newlander on gamma turn mimetics the synthetic method varied depending on the sidechain sequence required—a 10 step sequence for a Gly-Phe-Leu mimetic, 13 steps for Phe-Gly-Val and 21 steps for Ala-Phe-Ala (Huffman, Callahan et al. 1988; Callahan, Bean et al. 1992; Newlander, Callahan et al. 1993). Given that the possible combinations of three residue sequences of the 20 natural amino acids is 8000 (20×20×20), and 160,000 for the four residue beta turn sequence, such non-generic methods are of limited use. The methods of Callahan and Huffman were further hampered by a lack of chiral control, as are most methods in the art.

In the development of peptide turn mimetics a further important issue is the reproduction of the variety of different turn conformations, particularly of the beta turn. Several different methods of describing turn conformation have been proposed, the traditional method having several turn types based on the backbone dihedral angles of the (i+1) and (i+2) residues i.e. I, I′, II, II′, III, III′, IV, V, VIa, VIb, VII and VIII, with even this diversity of types being insufficient to adequately describe turn conformations (Richardson 1981; Wilmont and Thornton 1990; Ball, Hughes et al. 1993). No single mimetic framework can accurately mimic this diversity of turns; a selection of mimetic frameworks is required.

The problems encountered in the development of peptide turn mimetic syntheses are discussed in a review by Kahn (Kahn 1993) and reference may also be made to a review article entitled “Design of Peptidomimetics” (Nakanishi and Kahn 1996) which discusses aspects of mimetic design and developments regarding peptide mimetics.

The uses of reverse turn mimetics (and peptides or other compounds containing reverse turn mimetics) in drug development have been described in the art, notably in publications by Kahn and co-workers (Kahn 1996; Nakanishi and Kahn 1996; Qabar, Urban et al. 1996) and references contained therein. An important example of the application of reverse turn mimetics is the production of mimetics of known biologically active cyclic peptides (typically penta- or hexapeptides), as illustrated by Hirschmann and co-workers with α-D-glucose based mimetics (Hirschmann, Nicolau et al. 1992; Hirschmann, Nicolau et al. 1993).

Other beta turn mimetics having biological activity are known in the art. For example U.S. Pat. No. 4,535,169 discloses a method for the synthesis of beta turn mimetics which can incorporate a functional substitution for the (i+3) sidechain (only), and Krystenansky et al. disclose a leucine enkephalin mimetic based on this method which had analgesic activity one third the potency of morphine (Krstenansky, Baranowski et al. 1982).

Reference may also be made to U.S. Pat. Nos. 5,475,085 and 5,618,914 and International Publication WO96/22304 (all Kahn, M) which describe methods for the synthesis of a range of reverse turn mimetics. These mimetics are all produced by a modular synthesis technique (that may be applied to solid phase synthesis) which involves amino acid derivatives and various dipeptide azetidinones synthesised by a variety of techniques. An important common step in all of the syntheses of these mimetics is the cyclisation reaction which involves the azetidinone as activated ester component. Conformational variation is introduced to these mimetics by the inclusion of a variable component (“X”) in the ring of the cyclic turn mimetics. It should be noted that with two exceptions (the parent mimetics which have X═NH and have a ten or eleven membered ring) the beta turn mimetics produced by these methods have ring sizes of twelve members and above. Such large rings allow many conformations with d>7 Å, the mimetic conformations are therefore biased away from the accepted definition of a beta turn (d less than 7 Å), or more importantly the conformations are biased away from the most common reverse turn conformations which have d in the range of 4.5 Å to 6 Å (Rose, Gierasch et al. 1985; Gardner, Nakanishi et al. 1993). Enkephalin mimetics have been made (Gardner, Nakanishi et al. 1993) and also mimetics of a loop of CD4 that inhibit binding of HIV gp120 and infection of human lymphocytes (Chen, Chrusciel et al. 1992). The synthetic methods described for the majority of these mimetics appear to be limited with respect to the possible functionality at the (i) and (i+1) positions, and indeed no mimetic with any functionality at the (i+1) position (other than —H=glycine=no sidechain) appears to have been described at this time.

Reference may also be made to International Publication WO97/15577 (Kahn, M) which describes the synthesis of bicyclic reverse turn mimetics and chemical libraries containing such reverse turn mimetics. While concise, the synthetic methods do not provide for control of chirality at all positions, and the degree of sidechain function generality is questionable at two of the four positions. Furthermore the structure of the mimetics means they are not able to be easily incorporated in a peptide sequence, nor do they reproduce the relative positioning of the sidechain groups in the ideal manner (each sidechain attachment position should ideally be separated by three covalent bonds, as in a peptide).

Reference may also be made to the turn mimetics of Virgilio et al. (Valle, Crisma et al. 1989; Virgilio and Ellman 1994; Virgilio, Schüjrer et al. 1996) that incorporate functionality at the (i+1), (i+2) and (i+3) positions (but not the (i) position), and that do not allow for incorporation of the mimetic in a peptide sequence (i.e. no amino and carboxy terminal groups in addition to the sidechains are present).

Reference may be made to U.S. Pat. Nos. 5,438,188 and 5,470,849 (Callahan and Huffman) that describe biologically active compounds containing gamma turn mimetics, providing further illustration of the general utility of reverse turn mimetics.

Reference may also be made to International Publication WO95/25120 that describes the use of turn mimetics in the synthesis of peptide vaccines for generating a protective immune response in warm blooded animals.

In the methods and mimetics of the aforementioned references several common problems are evident: limited numbers of sidechains are able to be reproduced, there is limited control of chirality in the syntheses and a limited range of sidechain functions could be included. In addition, many of the syntheses of turn mimetics described are relatively long and complex, even when not all the sidechain functions are included, for example the syntheses of certain enkephalin mimetics were in the range of approximately 15 to 21 steps (Gardner, Nakanishi et al. 1993). There is therefore still a need in the art for peptide mimetics that can incorporate a wide range of sidechain functions in all positions, that can be readily synthesised with control of chirality, and that have a wide range of conformations corresponding to those found in native peptides.

### OBJECT OF THE INVENTION

It is the object of the invention to provide novel compounds useful as, and useful for the synthesis of, conformationally constrained mimetics of biologically active peptides and proteins (peptide mimetics). In particular the invention provides new compounds and methods for the synthesis of new peptide reverse turn mimetics that can display a wide range of sidechain functions at all sidechain positions, can be incorporated in a peptide sequence, can be readily synthesised, and have a variety of conformations.

### SUMMARY OF THE INVENTION

This invention provides novel compounds useful for the synthesis of peptide mimetics, and describes the use of these compounds for the synthesis of novel reverse turn mimetics. The reverse turn mimetics of the invention have the general structure X wherein the variables are as defined herein.

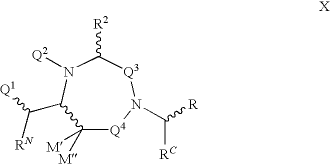

### BRIEF DESCRIPTION OF THE FIGURES

FIG. 1 shows the general structure of the mimetic systems and preferred cyclic turn and loop mimetic systems. Refer to the main text for a full description of the Q, R, Pg, Z and M groups.

FIG. 2 shows bicyclic beta turn mimetic systems. Refer to the main text for a full description of the R, Pg, Z and M groups.

FIG. 3 shows the structures of selected allylboron reagents.

FIG. 4 shows a NMR ROESY spectra of 210a R isomer (CDCl₃, 600 MHz)

FIG. 5 shows a NMR ROESY spectra of 210b S isomer (CDCl₃, 600 MHz)

FIG. 6 shows a comparison of Gal(1-16) 231 (first eluting peak), (R)-Gal(1-16) mimetic 232a (second eluting peak), and (S)-Gal(1-16) mimetic 232b (third eluting peak) peptides by analytical HPLC

FIG. 7 shows CD Spectra of Galanin Linear Reference Pentapeptide A (230=linear), (R)-Pentapeptide Helix Mimetic A (213a=R) and (S)-Pentapeptide Helix Mimetic A (213b=S): pH 7.4, 25° C.; buffer=10 mM phosphate buffer

FIG. 8 shows CD Spectra of Galanin(1-16) Linear Reference Peptide (231=Gal) and Galanin(1-16) Analogs Incorporating (R)-Helix Pentapeptide Mimetic A (232a=Gal R) and (S)-Helix Pentapeptide Mimetic A (232b=Gal S): pH 7.4, 25° C.; buffer=10 mM phosphate buffer, TFE=30% TFE in phosphate buffer

FIG. 9 shows Variable Temperature CD Spectra of Galanin(1-16) Linear Reference Peptide (231=Galanin) and Galanin(1-16) Analog Incorporating (S)-Helix Pentapeptide Mimetic A (232b=Gal S isomer): pH 7.4, 30% TFE in phosphate buffer

FIG. 10 shows CD Spectra of Temporin A V8G/I12L Linear Reference Peptide (234=linear) and Temporin A Analogs Incorporating (R)-Helix Pentapeptide Mimetic B (235a=R) and (S)-Helix Pentapeptide Mimetic B (235b=S): pH 7.4, 25° C.; buffer=10 mM phosphate buffer, TFE=30% TFE in phosphate buffer

FIG. 11 shows Variable Temperature CD Spectra of Temporin A V8G/I12L Linear Reference Peptide (234=Temporin), and Temporin A Analog Incorporating (S)-Helix Pentapeptide Mimetic B (235b=TA S isomer): pH 7.4, 30% TFE in phosphate buffer

FIG. 12 shows CD Spectra: Molar Ellipticity of Galanin(1-16) Linear Reference Peptide (231=Gal), Galanin(1-16) Analog Incorporating (S)-Helix Pentapeptide Mimetic A (232b=Gal S), Temporin A V8G/I12L Linear Reference Peptide (234=TA), and Temporin A Analog Incorporating (S)-Helix Pentapeptide Mimetic B (235b=TA S): pH 7.4, 30% TFE in phosphate buffer

### DETAILED DESCRIPTION OF THE INVENTION

The peptide mimetics of this invention have the general structure X, shown below and defined as follows:

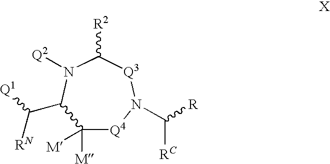

wherein R and R² and other R groups referred to hereinafter inclusive of R¹, R³, R⁴, Rⁿ⁺³ and Rⁿ⁺⁴ etc. unless otherwise indicated, are amino acid side chain groups, each independently chosen and therefore the same or different (two separate R groups in the same mimetic do not require a different suffix to indicate that they are independently chosen and can be the same or different). The definition of “amino acid side chain group” as used in this document is the same as the definition of “amino acid side chain moiety or derivative” as described in International Publication WO97/15577, pages 7-9 (Kahn, M), incorporated herein by reference. Amino acid side chain groups typically correspond to, but are not limited to, those found in natural amino acids and derivatives and in common unnatural amino acids. Thus for glycine R=hydrogen; for alanine R=methyl; for phenylalanine R=—CH₂Ph; for homophenylalanine R=—CH₂CH₂Ph; for valine R=—CH(CH₃)₂; for leucine R=—CH₂CH(CH₃)₂; p-nitrophenylalanine R=—CH₂((4-NO₂)Ph); naphthylalanine R=—CH₂-naphthyl etc. Also included are cyclic amino acid sidechains such as for proline, hydroxyproline and homoproline which involve a cyclisation to the adjcent backbone nitrogen atom or the equivalent position, but only where this is possible (i.e. the amine or equivalent atom is not already substituted as part of the heterocyclic mimetic framework).

Z is normally hydrogen, methyl, ethyl, formyl or acetyl, and may alternatively be R or —CH₂R or —C(O)R where R is an amino acid side chain group, or alternatively Z is part of a cyclic amino acid side chain group joined to R² (for example to mimic a proline residue at position (i+1)). For II(i), Z cannot be hydrogen due to compound instability.

R^{C} is the carboxy terminal part of the mimetic, typically —C(O)Pg^{C} or alternatively hydrogen or an amino acid side chain group R or —CH₂R.

Pg^{C} (and Pg^{C}′ etc.) is a protecting group for carboxylic acid, typically including, but not limited to: alkoxy, benzyloxy, allyloxy and fluorenyl methyloxy, amines forming easily removable amides, or alternatively an appropriate cleavable linker to a solid phase support, or such a support itself, or alternatively hydroxy or —OR or —NHR where R is an amino acid side chain group, or alternatively part or all of the remaining C-terminal portion of the mimetic system as described below.

R^{N} is the amino terminal part of the mimetic, i.e. —N(Z′)Pg^{N}.

Z′ is normally hydrogen, alternatively methyl (to mimic an N-methyl amino acid residue at position (i)), or alternatively part of a cyclic amino acid side chain group joined to R¹ (for example to mimic a proline residue at position (i)).

Pg^{N} (and Pg^{N′}) is a protecting group for amine, typically including, but not limited to: Boc, Cbz, Fmoc, Alloc, trityl; or alternatively an appropriate cleavable linker to a solid phase support, or such a support itself, or alternatively hydrogen or R or —C(O)R where R is an amino acid side chain group, or alternatively part or all of the remaining N-terminal portion of the mimetic system, as described below.

M, M′, M″ are normally hydrogen, alternatively one or more may be C₁-C₄ alkyl (preferred methyl), chloro, C₁-C₄ alkoxy (preferred methoxy).

Q¹=R¹ and Q²=Z; alternatively there is a cyclisation from Q¹ to Q² and then in preferred embodiments of the invention Q¹Q²=—CH(R)C(O)— or —CH₂CH(R)C(O)— or —CH₂CH₂CH(R)C(O)—. Q¹Q² can also be: —CH(R)CH₂— or —CH₂CH(R)CH₂— or —CH₂CH₂CH(R)CH₂— or —CH₂CH(R)— or —CH₂CH₂CH(R)— or —CH(R)CH₂CH₂— or —CH₂CH(R)CH₂CH₂— or —CH(R)CH₂C(O)— or —CH₂CH(R)CH₂C(O)—; Q³=—C(O)NHCH(R)Y— or —C(O)ENHCH(R)Y—;

Y is C(O);

Q⁴ is CHM′;

E=-(AA)ₙ- where n=1 and AA is an amino acid residue (e.g. AA=—NHCH(CH₃)C(O)— for alanine).

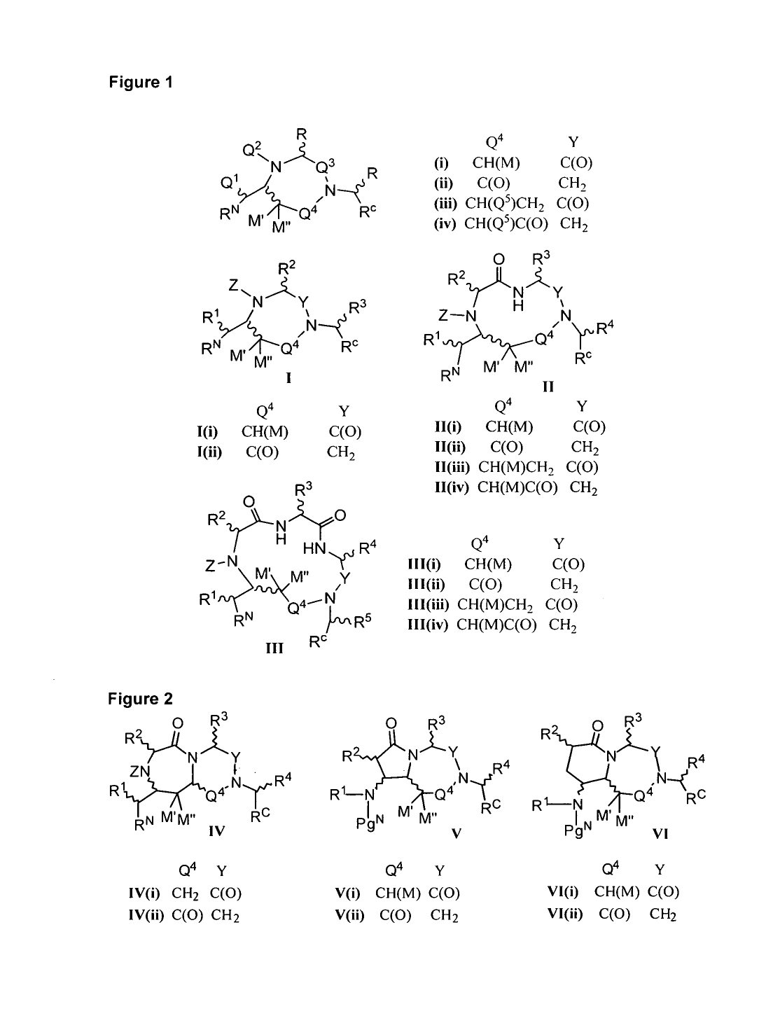

Preferred embodiments of the invention are the structures II-III, as illustrated in FIGS. 1 and 2 and defined in Table 1:

TABLE 1

| Mimetic | Q¹ | Q² | Q³ |
|---|---|---|---|
| II | R¹ | Z | —C(O)NHCH(R)Y— |
| III | R¹ | Z | —C(O)NHCH(R)C(O)—NHCH(R)Y— |

The compounds of this invention have been designed to allow for incorporation in a peptide or protein chain, or for covalent attachment to any molecule or group that may be useful for the enhancement of the biological activity, or other property, of the peptide mimetic. Thus the mimetics typically contain amino and carboxy termini independent of the sidechain functions. The term “remaining C- (or N-) terminal portion of the mimetic” is any group, molecule, linker, support, peptide, protein, nucleoside, glycoside or combination of these, covalently linked to the mimetic. Typically such remaining portions would be peptides or combinations of peptides and other mimetics, or compounds to facilitate detection or identification, or to improve the pharmacodynamics or other useful feature of the mimetic system.

In addition, any R group (an amino acid side chain group) may serve as an attachment point to a solid support, or to a linker to a solid support, or as a covalent attachment point for another molecule that may be useful for the enhancement of the biological activity, or other property, of the mimetic, as described above for the remaining C- or N-terminal portions of the mimetic.

An “easily removable amide” as the term is used herein with respect to Pg^{C} or Pg^{C}′ refers to an amide group that can be cleaved to yield the corresponding amine under conditions that do not degrade the substrate molecule.

The term “cleavable linker” and “solid phase support” are as defined in International Publication WO97/1557

The use of a wavy line for one of the bonds at a chiral centre in the general structures X and I-VI and in the other structures in the Figures and Schemes indicates that the centre may be in either the (R) or (S) configuration, or be a mixture in any proportion of the (R) and (S) configurations. In most circumstances it is preferable to avoid mixtures of configurations unless the intention is to provide a mixture of diastereomers for example for the purpose of more efficient screening (by the use of a mixture) or for synthetic expediency. Chirality at the amino acid side chain positions in the compounds of the invention (e.g. at R¹ to R⁴) is controlled by the use of chiral starting materials (L or D amino acids) and the avoidance of synthetic conditions which cause racemisation. The configuration at chiral centres formed in the mimetic synthesis is dependent on several factors and can be controlled in several cases, but in other cases mixtures of diastereomers will result, which can potentially be separated by physical means. A significant advantage of the invention is the superior level of chiral control possible at the chiral centres in the mimetics.

### EXAMPLES OF PREFERRED EMBODIMENTS OF THE MIMETICS

β-Turn mimetics III(i)a (M, M′, M″ and Z′=hydrogen, Z=H or Me):

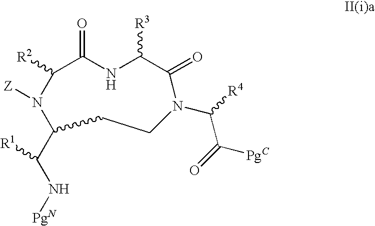

β-Bulge mimetics III(i)a, (M, M′, M″ and Z′=hydrogen, Z=H or Me):

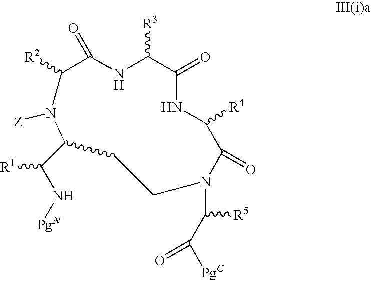

The synthesis of all the mimetics described in this specification may proceed initially by the same general synthetic procedure for formation of the common intermediates—reaction of imines 3 with allyl metal reagents Rg¹ (allyl boranes preferred) to give the allyl diamines 4, which are new, as described in Scheme 1. The other compounds of Scheme 1 (i.e. 5-8) may all be derived from the allyl diamines 4, as described in Scheme 1 and in the comments below. The allylation reaction of imines 3, which falls within the scope of the invention, is remarkable for its mildness and selectivity—allowing a wide range of functional groups to be present in the rest of the molecule, a very important consideration in the synthesis of peptide mimetics. Another important feature of the reaction of allylboranes with the imines 3 is that it proceeds in good yield (e.g. >50% isolated yield) in the sterically hindered general case where R¹ and R² are both not hydrogen—i.e. for all mimetics of dipeptides not containing glycine. Scheme 1 and all subsequent Schemes describe the preferred case of R^{N}═NHPg^{N} and R^{C}═C(O)Pg^{C} (FIGS. 1 and 2), analogous methods apply in the general case.

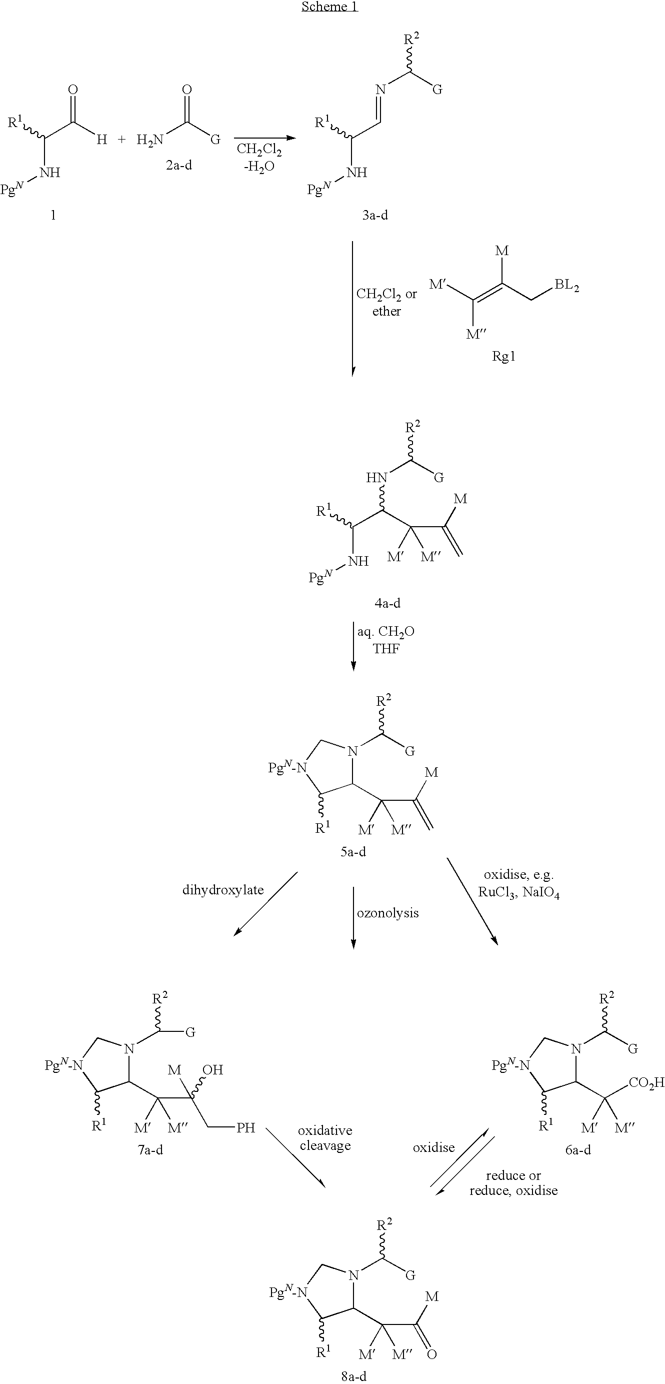

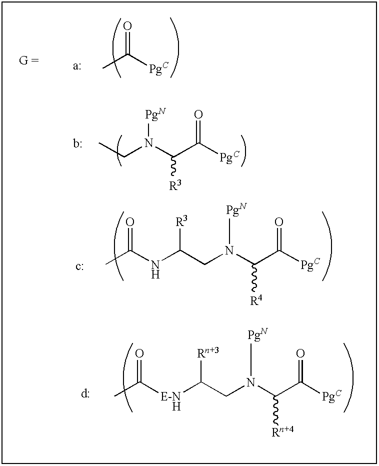

In relation to Scheme 1, preparation of the imines 3 is completed by condensation of an amino acid aldehyde (compound I) with an amine (2a-d). The aldehydes 1 may be prepared by either oxidative procedures from the corresponding N-protected amino alcohol, or reduction of an N-protected amino acid derivative (Fehrentz and Castro 1983), the different approaches have been reviewed, (Jurczak and Golebiowski 1989) (see also Goel et al., Org. Syn. 67:69, 1988). The amines 2a are amino acid esters (or other acid protected amino acid derivatives), which are commercially available or may be synthesised by standard procedures from amino acids. Amines 2b-2d are prepared by reductive amination of an amine 2a and an amino acid aldehyde 1:

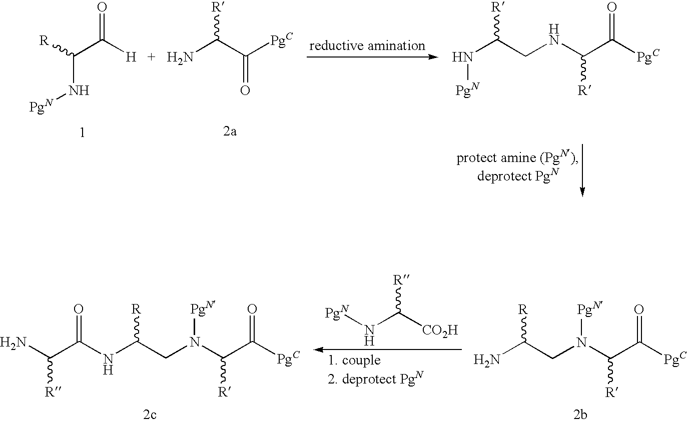

Amines 2d are prepared by repeated coupling/deprotection steps (as in conversion of 2b to 2c), standard techniques of peptide synthesis.

The reductive amination procedure for the alkylation of amines by aldehydes is well established in the art (see for example Sasaki and Coy, Peptides, 8:119, 1987), the preferred reagents are sodium cyanoborohydride (Borch, Bernstein et al. 1971; Hutchins and Natale 1979; Gribble and Nutatits 1985), or more preferred sodium triacetoxyborohydride in dichloroethane (Abdel-Magid, Carson et al. 1996).

Methods for the formation of amide bonds (coupling) are well established in the art. For coupling at more hindered amines the use of certain reagents, for example those based on 1-hydroxy-7-azabenzotriazole (Ehrlich, Rothemund et al. 1993; Carpino, El-Faham et al. 1994), or the use of amino acid fluorides (Carpino, Sadat-Aalaee et al. 1990; Wenschuh, Beyermann et al. 1994) is advantageous.

Protecting strategies for the synthesis of peptides and peptide mimetics are well established in the art, for example a five dimensional orthogonal strategy was used by Hirschmann and co-workers in the synthesis of a somatostatin mimetic (Hirschmann, Yao et al. 1996). A more general reference work on protection/deprotection is the monograph by Greene and Wuts (Greene and Wuts 1991).

The example syntheses described in this document use solution phase chemistry. The mimetics may also be synthesised by analogous solid phase techniques, or by a combination of solid phase and solution phase techniques, or the mimetics may be incorporated in normal solid phase peptide synthesis in the same way as a standard protected amino acid derivative. A review by Früchtel and Jung (Früchtel and Jung 1996) details the state of the art in solid phase organic synthesis (in 1996).

It will be clear to those skilled in the art that the mimetics of the invention, due to their generic methods of synthesis, are suited to the application combinatorial chemistry techniques (more specifically combinatorial organic synthesis) and certain associated identification and screening techniques. The application of combinatorial and associated technologies to drug discovery are well known in the art and have been reviewed, see for example papers by Gallop et al. and by Gordon et al., and references therein, incorporated herein by reference (Gallop, Barrett et al. 1994; Gordon, Barrett et al. 1994). Additionally, reference may be made to a review by Thompson and Ellman on the synthesis and application of small molecule libraries, and references therein, incorporated herein by reference (Thompson and Ellman 1996).

The imines 3 form rapidly at room temperature on mixing of the amine and aldehyde in an appropriate solvent, e.g. CH₂Cl₂ or diethyl ether, with liberation of water. The water is removed with a drying agent, e.g. dried MgSO₄, which is subsequently removed by filtration. The imines are then reacted with an allyl metal reagent (Rg¹) to give, after work-up, compounds 4 (Scheme 1).

In relation to reagents Rg1: standard allyl organometals, such as allyl magnesium bromide, are unsuitable for reaction with imines 3 due to a lack of selectivity for the imine function over the carboxylic acid derived groups (esters, amides) also present in 3. Allyl copper and zinc reagents have been used in selective reactions with imines (Bocoum, Boga et al., 1991; Basile, Bocoum et al., 1994) but in the case of imines 3 these reagents result in extensive racemisation at the α-imine chiral centre, and attack esters present in the imine to a significant extent. While some of the desired target 4 may be produced by many allyl metal reagents on reaction with 3, the reaction product typically contains a mixture of four diastereomers and also by-products from reaction at the carboxylic acid derived groups (especially esters). In contrast to these results, reaction of the imines 3 with allyl boranes, such as B-allyl-9-borabicyclo[3.3.1]nonane (allyl-9-BBN), Rg1a, gives excellent results and reasonable diastereoselectivity (>50% isolated yield based on crude aldehyde, and ˜80:20 diastereoselectivity where R¹ is not H).

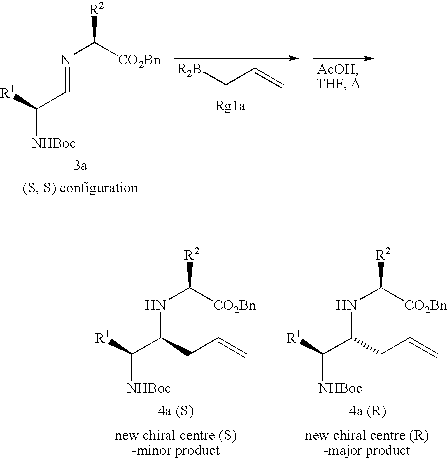

By the use of allyl trialkylboranes with appropriate chiral alkyl groups such as B-allyl-diisopinocampheylborane (allyl-DIP, Rg1b and Rg1c), or the diisocaranylboranes Rg1d-e it is possible to produce only the major product (one diastereomer, >99:1) in good yield and purity. The configuration at the new stereocentre was determined to be (R) when using aldehyde derived from natural (S) configuration amino acids, and the stereocontrol exerted by the α-aldehyde chiral centre was dominant over the effect of chiral boron ligands and over the effect of the other amino acid chirality in all cases examined. The (+)DIP reagent Rg1b gave higher diastereoselectivity on imines derived from natural (S) configuration aldehydes than Rg1c (from (−)DIP). The purity of the allylation products 4a may also be improved by the removal of the ester protecting group Pg^{C} to give a crystalline amino acid which can be recrystallised (e.g. from ethanol/water) to the desired level of purity and then reprotected.

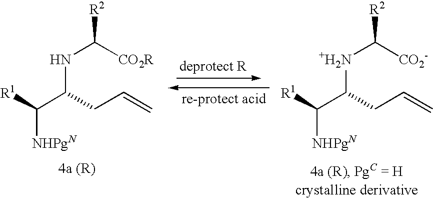

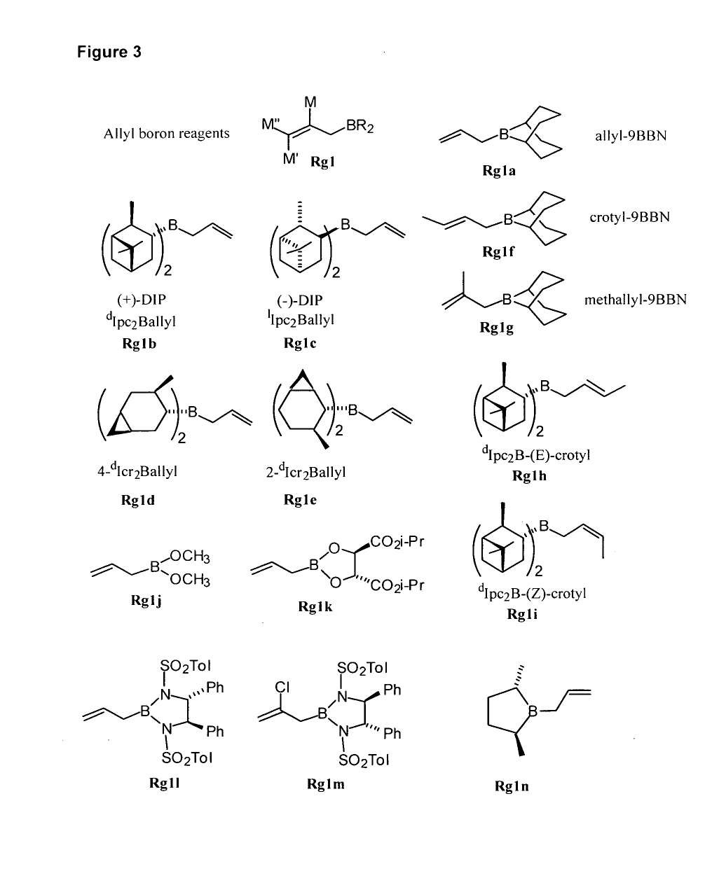

The use of crotyl (Rg1f, Rg1h-i), methallyl (Rg1g) or other substituted allyl derivatives leads to bridge substituted mimetics (mimetics where at least one of M, M′ and M″ is not hydrogen) with further opportunities for stereocontrol. The less reactive allyl boronate allyldimethoxyboron (Rg1j) was found to give inferior results (significant epimerisation at Cα(i)) compared to the allyltrialkylboranes. Many allylboronate and related reagents (e.g. Rg1k-m) are described in the literature, and some of these may be more effective than allydimethoxyboron for the conversion of 3 to 4. Selective reactions using allylic metals have been reviewed by Yamamoto and Asao, Tables IV and V in the review (pp. 2224-2230) list a wide variety of allyl boron reagents (Yamamoto and Asao 1993). The preparation of allyl-9-BBN and other allyltrialkylboranes has been described by Brown and coworkers (Kramer and Brown 1977; Brown and Jadhav 1983; Brown and Jadhav 1984; Brown and Bhat 1986; Brown, Randad et al. 1990). Allyltrialkylboranes may also be prepared by the reaction of the corresponding B-chloro or B-methoxy derivative with an allylmagnesium bromide (−78° C., diethyl ether), and reacted in situ with the imine (Yamamoto and Asao 1993). The imines 3 formed from two non-glycine derivatives (i.e. R¹ and R² not H) are significantly hindered about the imine nitrogen, and the use of bulky boron ligands (such as diisopinocampheyl) can reduce the reaction yield. For high yield and selectivity smaller chiral B-allyl compounds, e.g. those based on 2,5-dimethylboracyclopentane are preferred (e.g. Rg1n, FIG. 3).

In relation to protection and deprotection of compounds 4 and 5: addition of formaldehyde solution to 4 results in the rapid formation of imidazolidines 5; the relative configuration in the major allylation products 4 results in a 4,5-cis-substituted imidazolidine 5. This protection strategy is important for further reaction of these compounds. The protecting group is removed by treatment with aqueous acid (e.g. aqueous methanolic acetic acid).

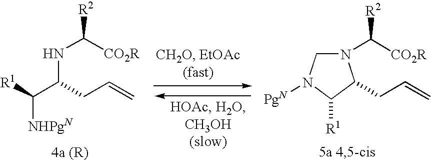

A similar protection system is the dibenzyltriazone group of Knapp and co-workers (Knapp, Hale et al. 1992), the paper describes other deprotection conditions and is incorporated herein by reference. An alternative deprotection method involves the hydrogenation of the imidazolidine system to an amine N-methyl group (40 psi H₂, Pd—C, MeOH, 48 hrs), a conversion that can be used to give mimetics where Z=Me.

In relation to oxidation of alkenes 5: acids 6 can be synthesised directly by oxidative cleavage of the alkenes 5, e.g. by RuCl₃/NaIO4; aldehydes/ketones 8 may be synthesised directly from 5 by ozonolysis (for oxidation methods see for example the monograph by Hudlicky (Hudlicky 1990) and references therein), but this process is not sufficiently selective and results in over-oxidation and the formation of other by-products. Preferred is the two step process of dihydroxylation (OsO₄, N-methylmorpholine-N-oxide (NMO),tBuOH/water) (VanRheenen, Kelly et al. 1976; Ray and Matteson 1980) to 7 followed by oxidative cleavage (Pb(OAc)₄ in benzene or H₅IO₆ in THF) (Hudlicky 1990). Examination of the products of the oxidation reactions led to the surprising discovery that cleavage with Pb(OAc)₄ resulted in isomerised product with the 4,5-substituents now trans, not cis as in the starting material. It was further discovered that oxidation of the diol with H₅IO₆ in dry THF resulted in retention of the 4,5-cis configuration in the aldehyde product 8. The cis aldehydes can also be isomerised to the trans by treatment with catalytic acid, e.g. HCl in CHCl₃.

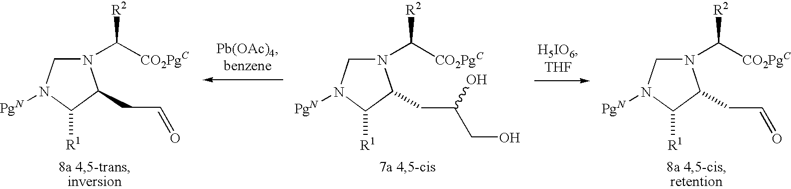

These important discoveries now allow selective access to all of the eight possible diastereomers of the aldehydes 8 and the acids 6, and therefore control of the majority of the chirality in all the mimetic systems described in the invention.

In relation to the oxidation of aldehydes 8 to acids 6: many oxidation reagents may effect this conversion, e.g. pyridinium dichromate (Hudlicky 1990). Glycols 7 may also be oxidised directly to acids, e.g. by RuCl₃/NaIO₄. In relation to reduction of acids 6 to aldehydes 8: carboxylic acids 6 can be converted to aldehydes by the same general methods used for the formation of protected α-amino aldehydes described above (Jurczak and Golebiowski 1989). The carboxylic acid can be selectively reduced to the alcohol in the presence of carboxylic esters by the use of borane (Brown and Krishnamurthy 1979), then oxidised to the aldehyde as previously described (Jurczak and Golebiowski 1989).

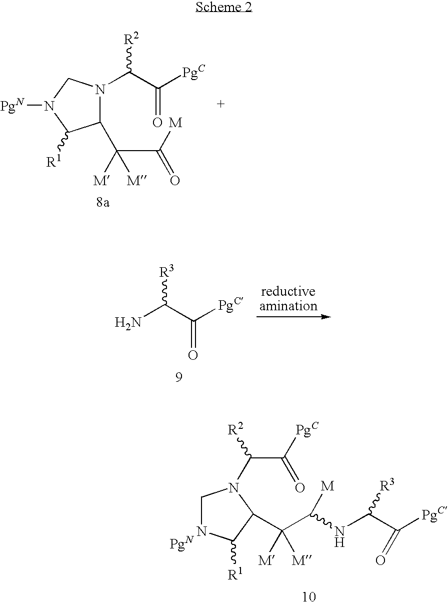

In relation to Scheme 2: Aldehydes/ketones 8 undergo reductive amination with amino esters 9 by the methods previously described. The preferred method is NaBH(OAc)₃ in dichloroethane (room temperature). Surprisingly, it was discovered that the reductive amination of 4,5-cis imidazolidine aldehydes 8 resulted in the formation of the 4,5-trans amines 10 (−9:1 trans:cis). This isomerisation reaction is rapid (much faster than that of aldehydes 8) as the reductive amination reaction is complete in only a few minutes. It was further discovered that the isomerisation reaction could be prevented by the pre-formation of the imine between the aldehyde 8 and amine 9 (in MeOH, 2-4 h at room temperature) with rigorous exclusion of acid, followed by reduction with sodium borohydride to give the cis amine 10 from the cis aldehyde. This discovery allows the selective synthesis of either the 4,5-cis diastereomer or 4,5-trans (9:1 with cis) diastereomer of the amines 10 starting from the 4,5-cis aldehyde 8.

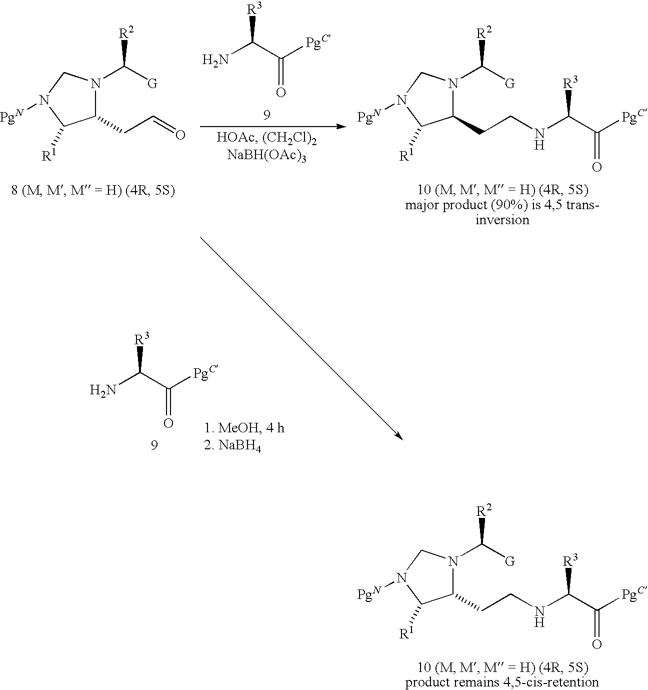

It is important to appreciate that the methods described above allow the selective synthesis of all sixteen relative and absolute diastereomers of compounds 8 and 6, and all thirty two diastereomers of compounds 10. The ability to selectively synthesise these diastereomers is a significant advantage of the invention.

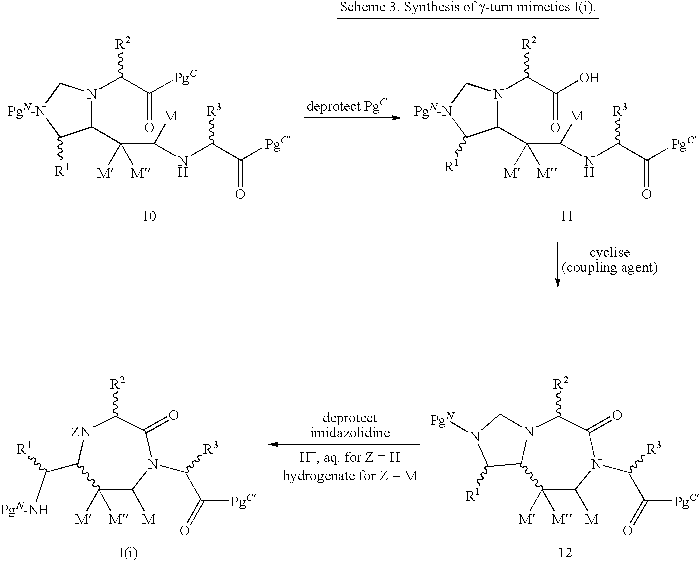

In relation to Scheme 3: Deprotection of 10 is by standard methods consistent with the overall protecting strategy, as previously discussed. Many coupling agents are suitable for effecting the cyclisation of 11 to 12, typical conditions: THF, BOP or HBTU or HATU, EtN(i-Pr)₂ (DIEA). The imidazolidine group is then deprotected (as previously described) by hydrogenation (MeOH, H₂—Pd/C) when Z=Me, and by hydrolysis (H⁺, H₂O) for Z=H (other Z groups may be introduced by acylation or alkylation of the deprotected secondary amine).

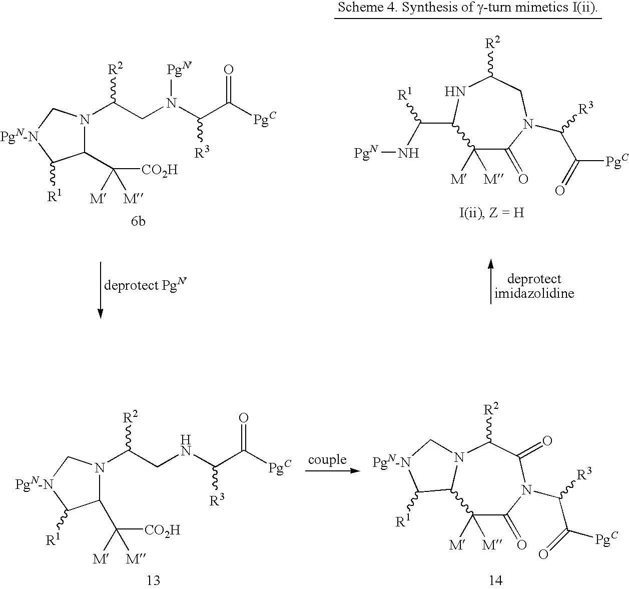

In relation to Scheme 4: Deprotection and cyclisation of 6b to 13, 14 and I(ii):—standard deprotection and coupling (cyclisation) methods are used. Other conversions are as previously described.

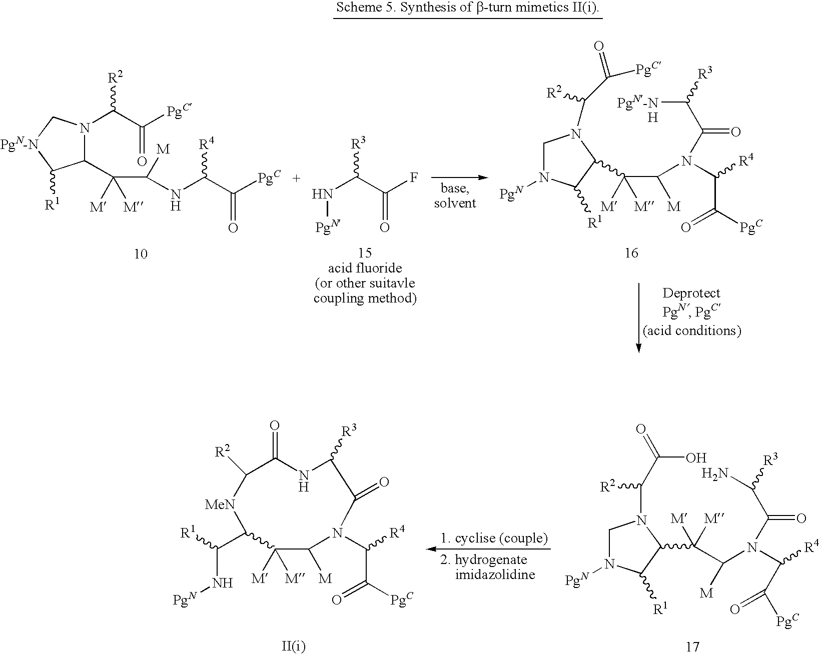

In relation to Scheme 5: As previously discussed, coupling reactions to relatively hindered (usually secondary) amines often require the use of specialised coupling conditions such as acid fluorides 15, as described by Carpino et al. (Carpino, Sadat-Aalaee et al. 1990; Wenschuh, Beyermann et al. 1994) Protecting groups Pg^{N′} and Pg^{C′} (in 16) are typically benzyloxycarbonyl (Cbz) and benzyl ester, simultaneously deprotected by hydrogenation (0.1M HCl in EtOH, H₂—Pd/C), cyclised using the BOP coupling reagent in THF or DMF, followed by conversion (deprotection) of the imidazolidine group to N-Me by hydrogenation as previously described.

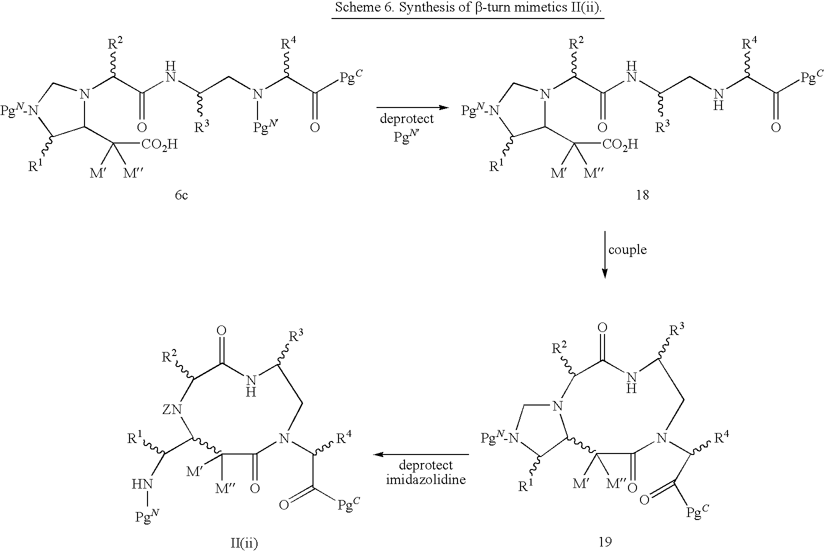

In relation to Scheme 6: Standard deprotection/coupling conditions as previously described.

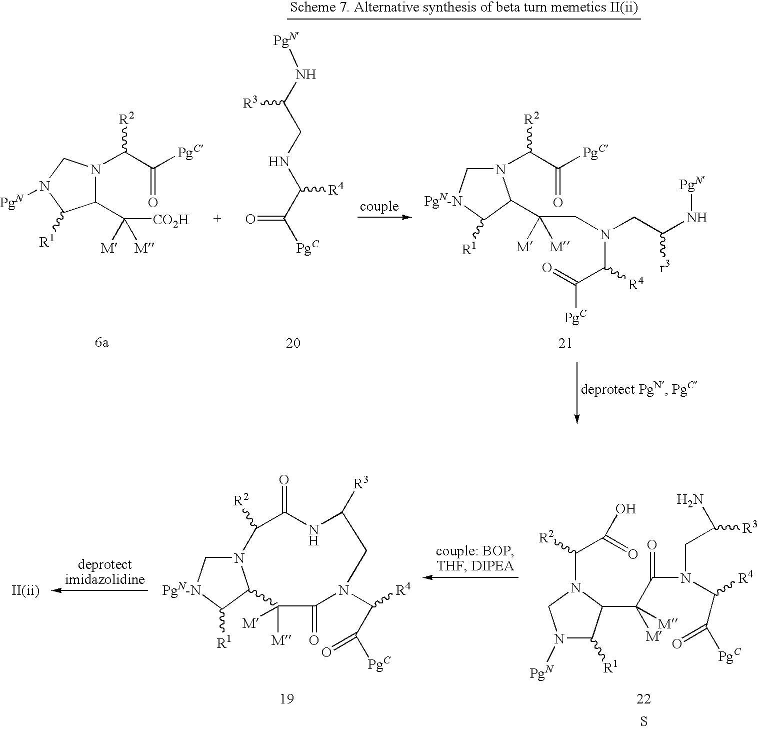

In relation to Scheme 7: Where R⁴ is a β-branched amino acid side chain (such as in Valine) then the coupling of 6a and 20 may require the use of HATU or other system suitable for a hindered coupling when bulky sidechain groups are present, as previously discussed. Conditions and protecting groups for the conversion of 21 to 19 are the same as for the conversion of 16 to II(i), Scheme 5.

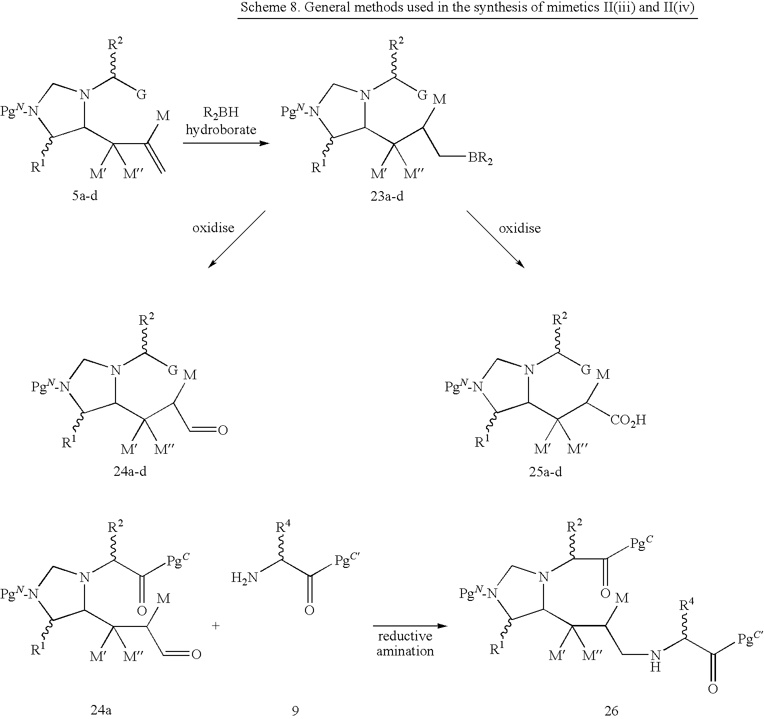

In relation to Scheme 8: Hydroboration of alkenes is well known in the art, see for example monographs by Brown (Brown 1975; Pelter, Smith et al. 1988). The resulting alkyl boranes can be oxidised to alcohols (using alkaline hydrogen peroxide, or in a preferred embodiment using trimethylamine oxide, or other amine oxide, to form the borate with subsequent liberation of the alcohol by transesterification) (Soderquist and Najafi 1986). Alternatively, treatment of the borane with acid dichromate or, in a preferred embodiment, with pyridinium chlorochromate (PCC) gives the aldehyde (Brown, Kulkarni et al. 1980; Brown and al. 1986). The aldehydes so formed may be reductively aminated on to amines 9 by the methods previously described.

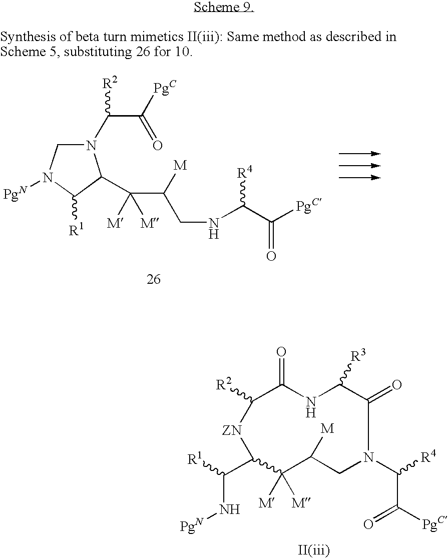

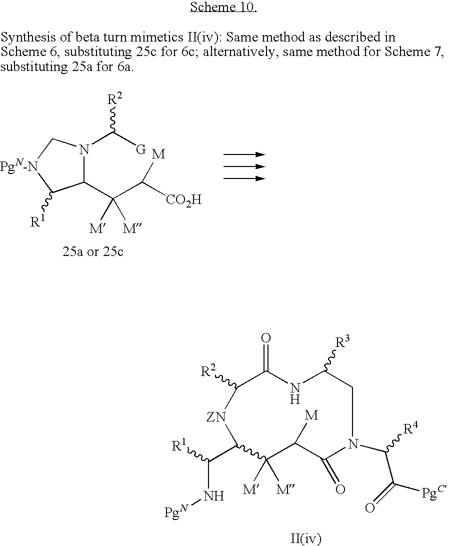

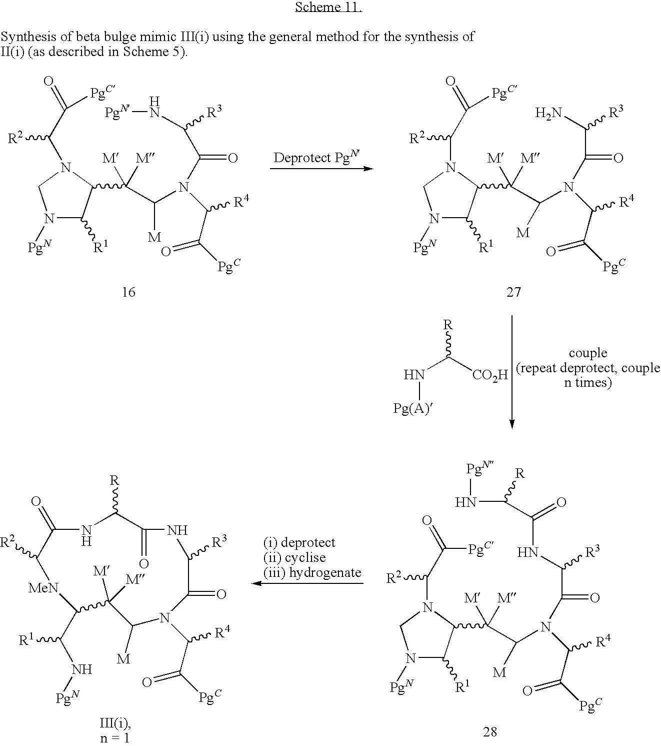

In relation to Schemes 9-11: Standard synthetic techniques, previously described.

Methods for the synthesis of beta bulge (n=1, III(i-iv)) and higher loop mimetics (n>1), follow the corresponding methods for the synthesis of beta turn mimetics II(i-iv). Appropriate protecting groups are chosen so that extra residues can be added to the system prior to cyclisation, as illustrated in Scheme 11 for the synthesis of a III(i) mimetic.

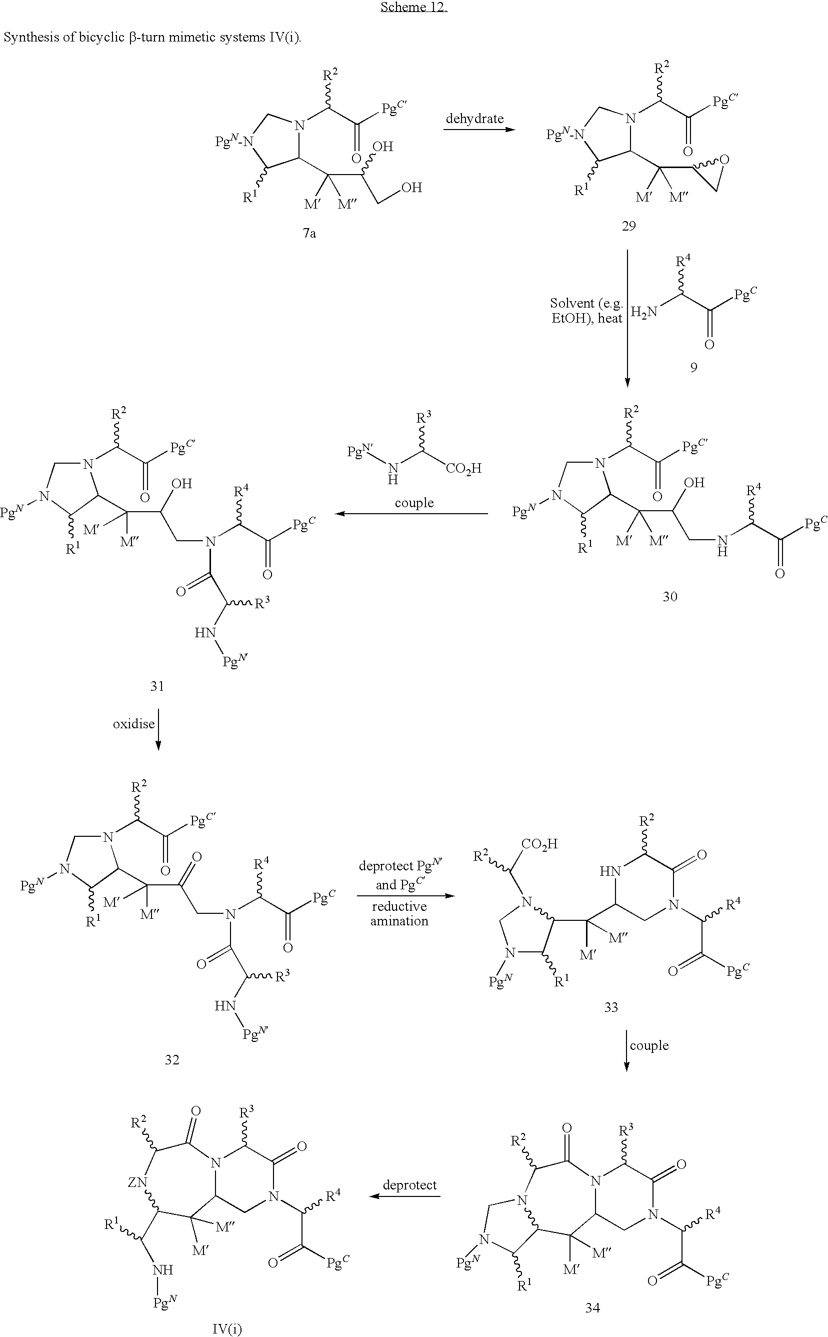

In relation to Scheme 12: Conversion of 1,2-diols 7 to epoxides 29 (dehydration) may be achieved with a number of reagents, for example triphenylphosphine and a dialkylazodicarboxylate (the Mitsunobu reagents) (Carlock and Mack 1978; Robinson, Barry et al. 1983) or TsCl/NaOH/PhCH₂NEt₃⁺Cl⁻ (Szeja 1985). The epoxides 29 alkylate amines 9 on warming in ethanol or DMSO solution to give the amino alcohols 30. The alcohol may then be oxidised to the ketone 32 by the use of TPAP (tetrapropylammonium perruthenate) with N-methylmorpholine-N-oxide in CH₂Cl₂/acetonitrile by the method of Griffith and Ley (Griffith and Ley 1990). For 32 typically Pg^{N′}=Cbz and Pg^{C′}=O-benzyl, then by the use of catalytic hydrogenation conditions (EtOH, H₂—Pd/C) the protecting groups are both removed and intramolecular reductive amination of the free amine to the ketone occurs to give 33. Coupling using the BOP reagent (or other suitable conditions) followed by deprotection of the imidazolidine group as previously described gives the bicyclic mimetic IV(i). Alternative syntheses are possible with the use of mild oxidising reagents to convert the glycols to carbonyl compounds, followed by reductive amination (Frigerio and Sangostino 1994).

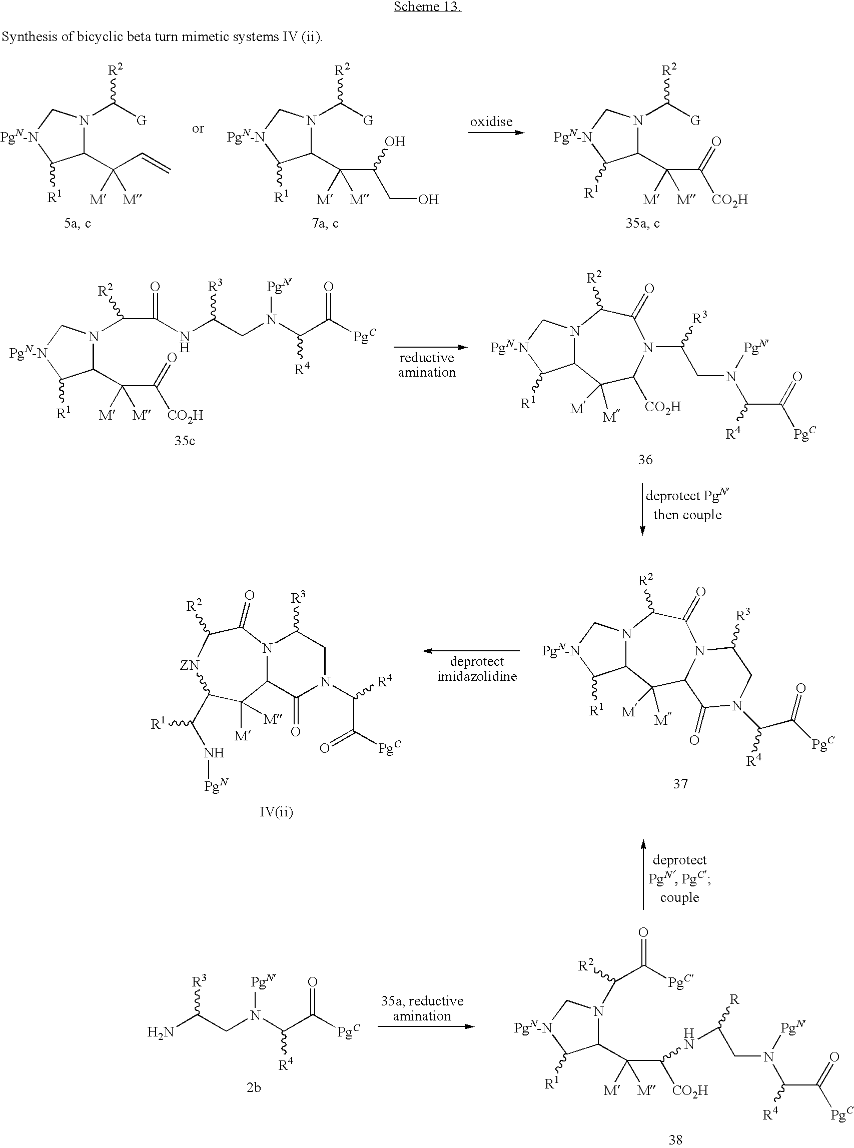

In relation to Scheme 13: 1,2 diols can be oxidised without carbon-carbon bond cleavage by the use of certain mild reagents e.g. IBX (Frigerio and Sangostino 1994). Conversion of 35c to 36 proceeds by intramolecular reductive amination, or alternatively 35a can be reductively aminated onto 2b, as indicated. Reductive amination, coupling and deprotection details are as previously described.

The syntheses for the bicyclic β-turn mimetic systems V and VI are accomplished from the corresponding γ-turn mimetic systems I, where the R¹ side chain group is derived from an aspartic acid (V) or glutamic acid (VI) derivative.

| | |
|---|---|
| | V |
| 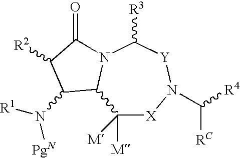 | |
| | VI |
| 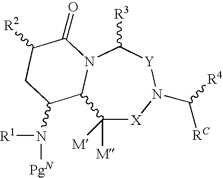 | |

| | X | Y |
|---|---|---|
| V(i) | CH(M) | C(O) |
| V(ii) | C(O) | CH₂ |
| VI(i) | CH(M) | C(O) |
| VI(ii) | C(O) | CH₂ |

The synthesis of mimetics V and VI thus proceeds as in Scheme 1, with the aldehyde component 1 (Scheme 1) being of the form 1d or 1e (Scheme 14), with the R and Pg groups as previously defined. The synthesis follows the synthesis of γ-turn mimetic systems I, and is completed by the method illustrated in Scheme 15.

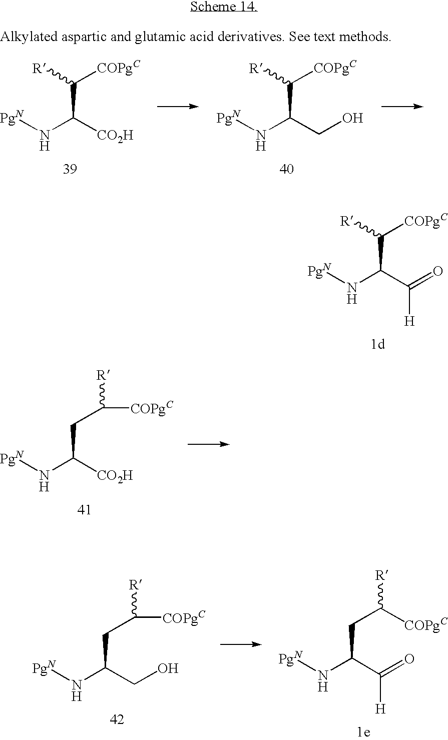

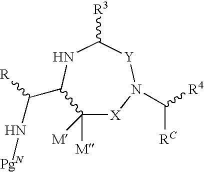

I (Z = H)

| | X | Y |
|---|---|---|
| I(i) | CH(M) | C(O) |
| I(ii) | C(O) | CH₂ |

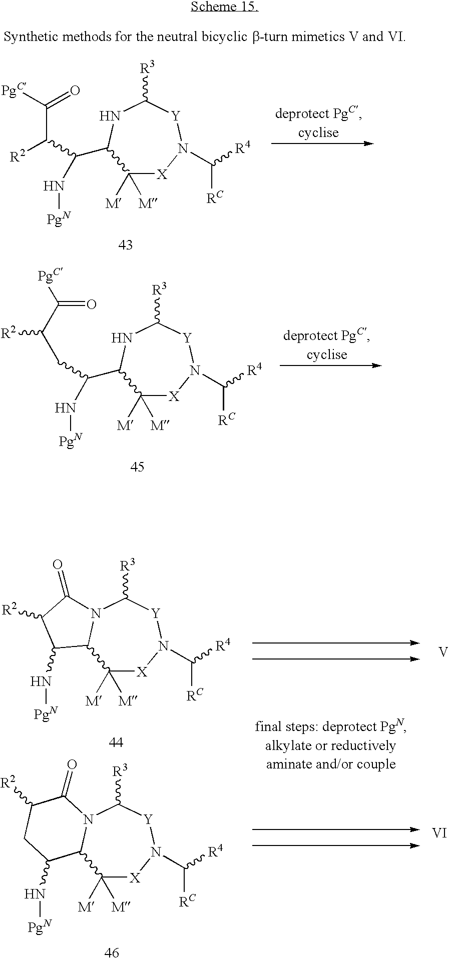

In relation to the preparation of alkylated aspartic and glutamic acid derivatives 1d and 1e the alkylated derivatives 39-42 can be prepared by a number of methods known in the art. Selected methods are summarised in Schemes 16 and 17. Rapoport and co-workers have developed methods for the selective alkylation of N-phenylfluorenyl protected aspartic and glutamic acid derivatives (Koskinen and Rapoport 1989; Wolf and Rapoport 1989). A review by Sardina and Rapoport, and references contained therein, describe several methods for the synthesis of alkylated aspartic and glutamic acid derivatives, incorporated herein by reference (Sardina and Rapoport 1996). Derivatives 39-42 are converted to aldehydes 1d and 1e by the methods previously described for the preparation of aldehydes 1.

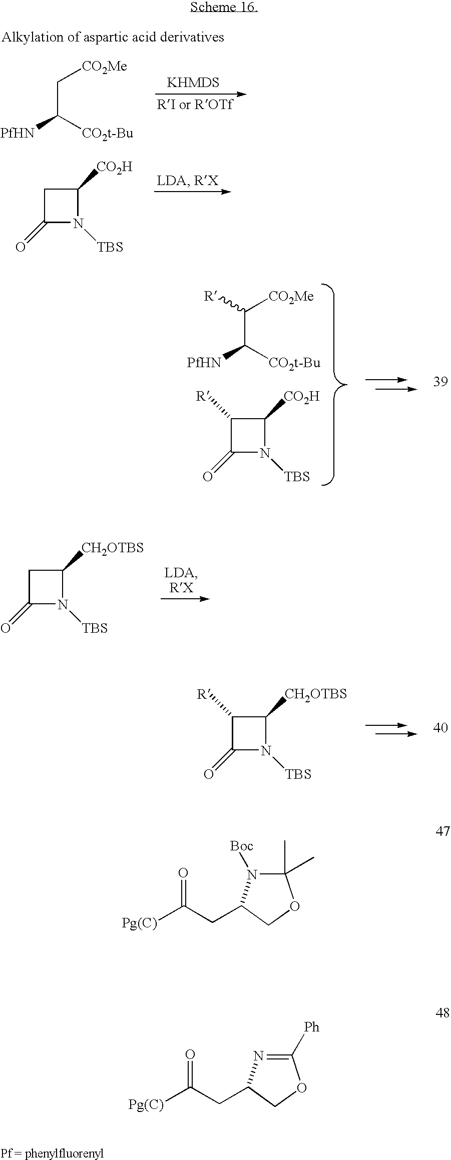

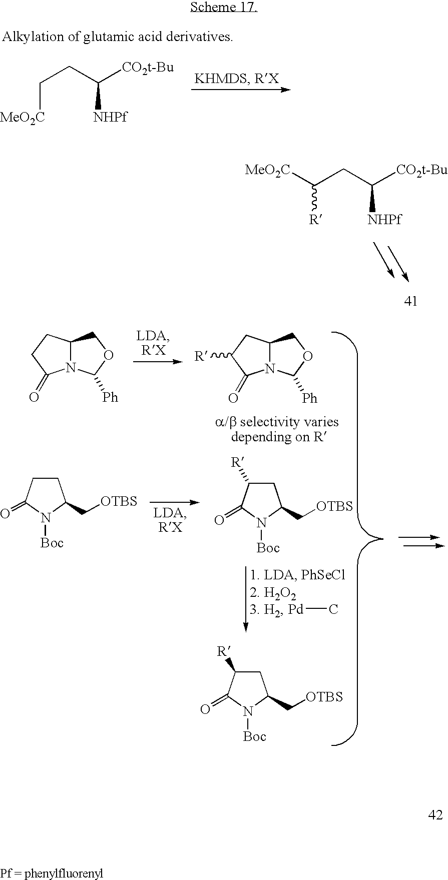

The use of standard chemical techniques, in particular the Arndt-Eistert homologation reaction (Meier and Zeller 1975) and reductions of carboxylic acids to aldehydes (Jurczak and Golebiowski 1989), and also the synthesis of ketones —C(O)R from amides —C(O)N(OMe)Me (Nahm and Weinreb 1981), to modify the aspartic and glutamic acid or their alkylated derivatives, or the use of similar derivatives of non-natural amino acids, such as homo-glutamic acid, enables the synthesis of the other compounds of the invention in which -Q¹Q²- (in the general structure X) forms part of a cyclic system, defined as: -Q¹Q²-=—CH₂CH₂CH(R)C(O)— (from sidechain alkylated homoglutamic acid); —CH(R)CH₂— (from aspartic acid by reduction of the γ-carboxylate and reductive amination); —CH₂CH(R)CH₂— (from glutamic acid by reduction of the δ-carboxylate and reductive amination); —CH₂CH₂CH(R)CH₂— (similarly from homoglutamic acid); —CH₂CH(R)— (from an aspartic acid sidechain ketone —CH₂C(O)R by reductive amination); —CH₂CH₂CH(R)— (from a glutamic acid sidechain ketone —CH₂CH₂C(O)R by reductive amination); —CH(R)CH₂C(O)— (post-alkylation sidechain homologated aspartic acid); —CH₂CH(R)CH₂C(O)— (post-alkylation sidechain homologated glutamic acid); —CH(R)CH₂CH₂— or —CH₂CH(R)CH₂CH₂— (from reductive amination of reduced post-alkylation sidechain homologated aspartic acid or glutamic acid derivatives).

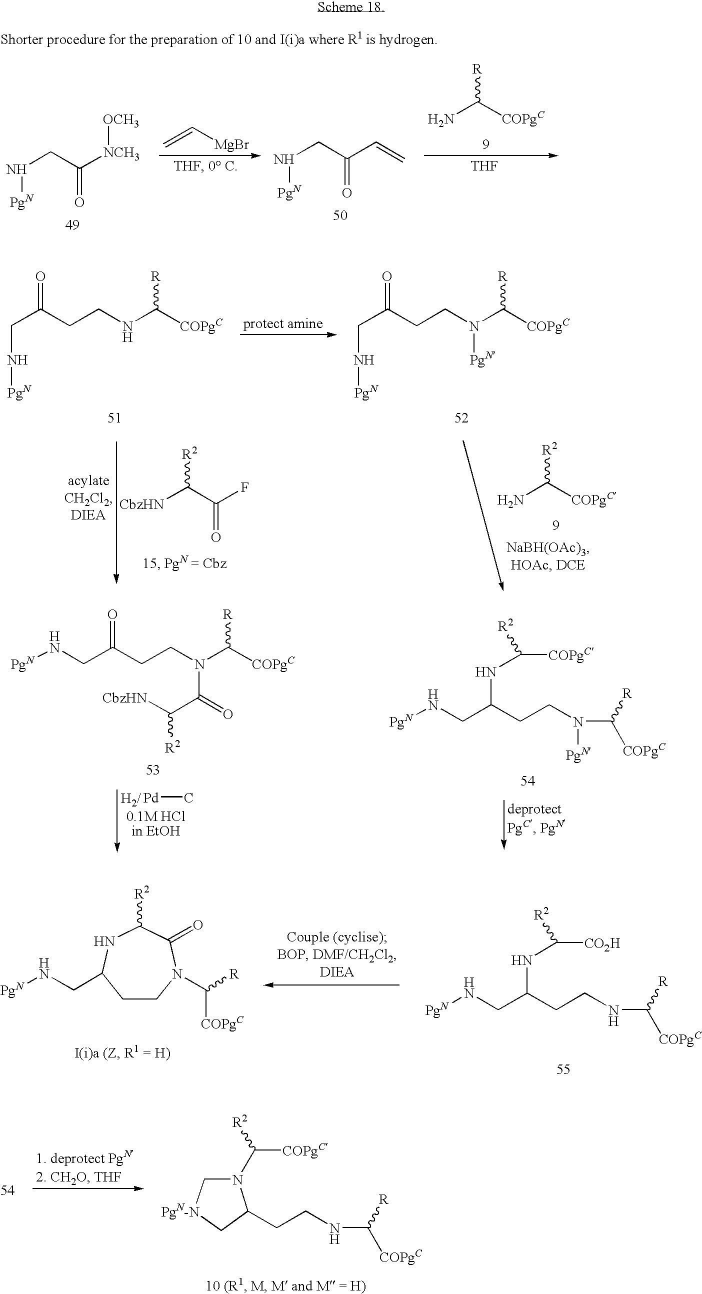

In relation to Scheme 18: An alternative procedure for the synthesis of intermediate compounds 10 (or equivalent) can be used in the case where R¹ is hydrogen and M, M′ and M″ are also hydrogen, as described in Scheme 18. Compound 49 is available commercially with certain N-protecting groups or can be made by coupling N-protected glycine with N,O-dimethylhydroxylamine. Reaction with vinylmagnesium bromide in analogy to the general procedure of Rapoport and co-workers (Cupps, Boutin et al. 1985; Boutin and Rapoport 1986) results in formation of the α,β-unsaturated ketone 50. Conjugate addition of an amino acid ester 9 (0° C., THF) results in the formation of aminoketones 51 which can be N-protected by standard procedures to form ketones 52 before reductive amination of an amino acid ester 9 under the conditions described by Abdel-Magid et al. (Abdel-Magid, Carson et al. 1996) (NaBH(OAc)₃, dichloroethane) to form 54. Deprotection to 55 and coupling gives the γ-turn mimetics I(i)a (where R¹═H) as indicated. Alternatively the aminoketones 51 can be acylated with an amino acid fluoride 15 to give compounds 53 which can be deprotected and cyclised (by reductive amination) by hydrogenation in mild acid conditions (H₂/Pd—C, 0.1M HCl in EtOH). The reductive amination-cyclisation is diastereoselective, only one diastereomer of the mimetics I(i)a was formed from 53, with the configuration at the new stereocentre controlled by the R² stereocentre. The (S) configuration at R² gives (S) at the new centre. In contrast, the reductive amination to form amines 54 proceeds with lower stereoselectivity (˜3:1) with the major diastereomer having the (R) configuration when R² is (S). These discoveries provide further opportunity for stereocontrol in the synthesis of the turn mimetics.

Deprotection of compounds 54 and reaction with formalin in THF is an alternative method for synthesis of compounds 10 (R¹═H), as described in Scheme 18.

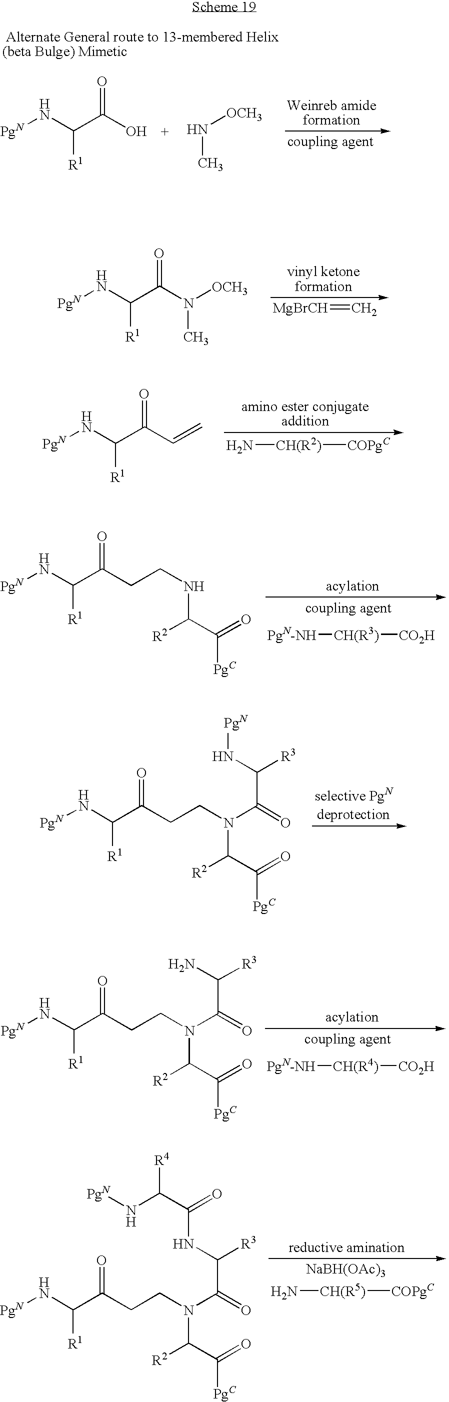

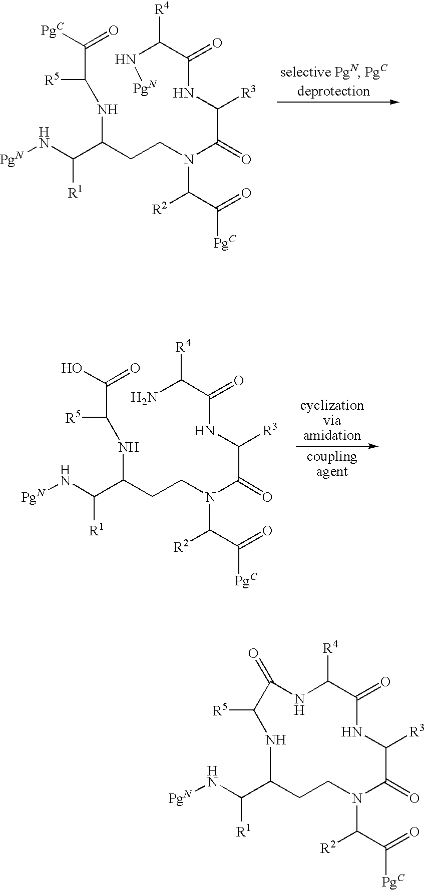

In scheme 19 there is disclosed an alternate general synthetic route to the claimed compounds of the invention (IIIa) having a 13 membered ring, forming a helix (beta bulge) mimetic. In Scheme 19, an N-protected amino acid derivative Pg^{N}—NH—CH(R¹)—CO₂H is converted to a Weinreb amide via activation of the carboxyl group and amidation with N-methyl methoxyamine. Addition of a vinyl Grignard reagent produces the aminoalkyl vinyl ketone, which undergoes conjugate addition by an amino ester H₂N—CH(R²)—COPg^{C}. The resulting secondary amine is acylated under standard peptide coupling conditions with an N-protected protected amino acid, Pg^{N}—NHCH(R³)—CO₂H. One Pg^{N} protecting group is selectively removed, and the free amine acylated with another N-protected amino acid Pg^{N}—NHCH(R⁴)—CO₂H. An amino ester H₂N—CH(R⁵)—COPg^{C} is employed for an intermolecular reductive amination of the ketone using standard reduction conditions, such as H₂/Pd catalyst, NaBH₄, NaBH₃CN, or NaBH(OAc)₃. Selective deprotection of one Pg^{N} and one Pg^{C} protecting group is followed by cyclization to form the 13-membered ring system via an intramolecular amidation reaction. There are many possible variations to this route, which include varying the order of introduction of the amino acid derivatives by altering the sequence of acylation/amidation reactions, varying the site of the intramolecular amidation reaction, or by varying the reaction sequence such that an intramolecular reductive amination is employed for the ring-closing step.

### Exemplary Syntheses

### Example (A)

### Synthesis of a γ-Turn Mimetic I(i) by the General Procedure

A mimetic for the sequence HTyr-Gly-Gly-Phe, which is found in the enkephalins, was synthesised with a γ-turn mimetic based on the Tyr-Gly-Gly tripeptide. Similar mimetics have shown activity at opiate receptors (Huffman, Callahan et al. 1988; Huffman, Callahan et al. 1989).

The synthesis is summarised in the following scheme:

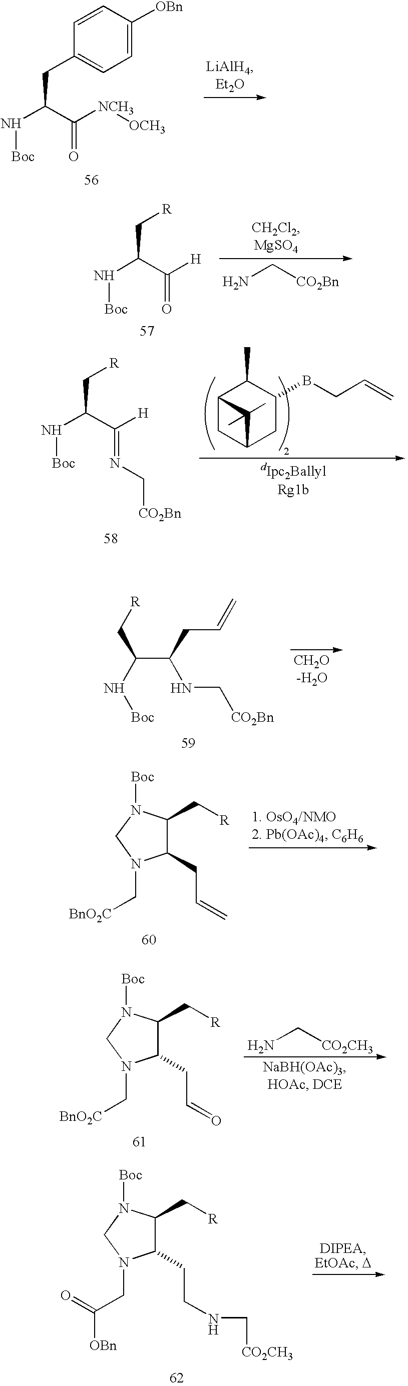

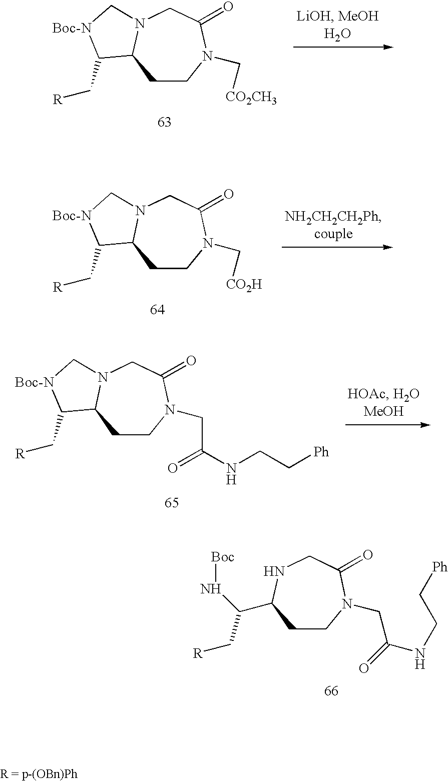

### Preparation of 56:

The amide 56 was synthesised from commercially available Boc-Tyrosine(OBn)OH by coupling with N,O-dimethylhydroxylamine hydrochloride, 1 equivalent, in DMF/CH₂Cl₂ (1:5) using HBTU reagent (1 eq.) and DIEA (2 eq.) at room temperature. The CH₂Cl₂ was evaporated in vacuo and the residue partitioned between diethyl ether and aq. NaHCO₃. The aqueous layer was separated and the ether layer washed in turn with 1M HCl (×2), aq. NaHCO₃, brine, and then dried over MgSO₄. Filtration and removal of the solvent in vacuo left the product amide 56 as a white crystalline solid in >90% yield. Further purification was carried out by silica gel chromatography eluting with ethyl acetate in petroleum ether, or by recrystallisation from ether. ¹H NMR (300 MHz, CDCl₃): δ 7.46-7.28, 5H, m, OBn; 7.08, 2H, d, J=8.5 Hz, Tyr Ar; 6.90, 2H, d, J=8.5 Hz, Tyr Ar; 5.15, bd, J=8 Hz, NH; 5.04, 2H, s, OCH₂Ph; 4.91, 1H, bm, Pheα; 3.65, 3H, s, OCH₃; 3.16, 3H, bs, NCH₃; 3.00, 1H, dd, J=6, 13.5 Hz, Pheβ; 2.83, 1H, dd, J=7, 13.5 Hz, Pheβ; 1.40, 9H, s, Boc. ¹³C NMR (75 MHz, CDCl₃): δ 172.3; 157.6, Tyr Ar—O; 155.1, carbamate; 137.0, ipso; 130.4; 128.8; 128.5; 127.8; 127.4; 114.7; 79.5, tBoc; 69.89, OCH₂Ph; 61.43, Tyrα; 51.55, OCH₃; 37.89, NCH₃; 32.00, Tyrβ; 28.26, Boc.

### Preparation of 57:

The aldehyde 57 was prepared by the method of Fehrentz and Castro (Fehrentz and Castro 1983) as follows: to a stirred solution of 4.2 g of amide 56 in 100 mLs of anhydrous diethylether cooled to 0° C. was added 0.51 g lithium aluminium hydride. After 10 minutes a solution of 1.5 g NaHSO₄ in 30 mLs of water was added. The reaction mixture was diluted with more ether and washed with 1M HCl, saturated aqueous sodium bicarbonate and brine and dried over magnesium sulphate. The volatiles were removed under reduced pressure to give a waxy solid which was recrystallised from cold ether/hexane to give 2.6 g (72%) of 57 as a white solid. ¹H NMR (300 MHz, CDCl₃): δ 9.62, 1H, s, aldehyde; 7.50-7.25, 5H, m, Ar(OBn); 7.10, d, J=8 Hz, Ar(Tyr); 6.93, 2H, d, J=8 Hz, Ar(Tyr); 5.10, 1H, b, NH; 5.05, 2H, s, OCH₂Ph; 4.39, 1H, q, J=7 Hz; Tyrα; 3.06, 2H, d(ABX), J=7 Hz, Tyrβ; 1.44, 9H, s, Boc. ¹³C NMR (75 MHz, CDCl₃): δ 199.6; 157.8, TyrOAr; 155.3, carbamate; 136.9, ipso; 130.3; 128.5, 127.9, 127.4: ArCH; 115.0, ArCHTyr; 80.08, tBoc; 69.69, OCH₂Ph; 60.82, Tyrα; 34.51, Tyrβ; 28.22, Boc.

### Preparation of 58:

The imine 58 was formed by the reaction of the aldehyde 57 (1.4 g) with one equivalent of glycine benzyl ester in 10 mL CH₂Cl₂ (stir at room temperature 1 h) the water formed was removed with magnesium sulphate which was then removed by filtration. ¹H NMR (300 MHz, CDCl₃): δ 7.68, 1H, s, imine; 7.49-7.30, 10H, Ar; 7.15, 2H, d, J=8 Hz, TyrAr; 6.92, 2H, d, J=8 Hz, TyrAr; 5.67, 1H, bd, J=6 Hz, NH; 5.20, 2H, s, OCH₂Ph; 5.05, 2H, s, OCH₂Ph; 4.51, 1H, bm, Tyrα; (4.26, 4.22), 2H, AB, J=15.5 Hz, Glyα; 3.15, 1H, bdd, J=5.0, 13.5 Hz, Tyrb; 2.93, 1H, dd, J=8.0, 13.5 Hz, Tyrb; 1.48, 9H, s, Boc. ¹³C NMR (75 MHz, CDCl₃): δ 169.3; 167.4, CH imine; 157.5; 155.1; 136.9, 135.3: 2× ipso; 130.4, CHAr; 128.8, Tyr ipso; 128.44, 128.39, 128.26, 128.19, 127.76, 127.29, 114.65: ArCH; 79.22, tBoc; 69.81, TyrOCH₂Ph; 66.60, GlyOCH₂Ph; 60.48, Tyrα; 54.73, Glyα; 37.97, Tyrβ; 28.23, Boc.

### Preparation of 59:

A 0.5 molar solution of allyl borane reagent ^{d}Ipc2Ballyl (Rg1b) was prepared by the addition of allylmagnesium bromide to one equivalent of (+)DIP-Cl in anhydrous diethyl ether under dry nitrogen (Brown and Jadhav 1983). The solution of imine 58 in CH₂Cl₂ was stirred and cooled to −78° C. under dry nitrogen and one equivalent of the previously prepared ^{d}Ipc2Ballyl solution added. The mixture was allowed to warm gradually to room temperature (overnight). The volatiles were removed under reduced pressure and the residue dissolved in THF and 1 mL of glacial acetic acid added. The mixture was refluxed overnight and then the volatiles removed under reduced pressure. The crude product was dissolved in CH₂Cl₂/petroleum ether and is the precipitate filtered off. The residual oil was chromatographed on flash silica eluting with ethyl acetate/petroleum ether to give 1.3 g (60% yield based on 57) of 59. TLC 1:2 EtOAc:light pet. Rf=0.40. ¹H NMR (300 MHz, CDCl₃): δ 7.48-7.30; 10H, Ar; 7.13, 2H, d, J=8.5 Hz, TyrAr; 6.91, 2H, d, J=8.5 Hz, TyrAr; 5.84, 1H, m, vinyl CH; 5.17, 2H, s, TyrOCH₂Ph; 5.16, 2H, m, vinyl CH₂; 5.05, 2H, s, GlyOCH₂Ph; 4.90, 1H, bd, J=8.5 Hz, NHBoc; 3.95, 1H, bm, Tyrα; 3.54, 2H, s, Glyα; 3.82, 1H, dd, J=4.5, 14.4 Hz, Tyrβ; 2.73, 3H, be: NH(amine), Tyrβ, CH(homoallyl); 2.28, 2H, m, allyl; 1.35, 9H, Boc. ¹³C NMR (75 MHz, CDCl₃): δ 172.1; 157.3; 155.6; 137.1, 135.4: ipso; 134.9, CHvinyl; 130.6, ipsoTyr; 130.0, 128.5, 128.4, 128.3, 127.8, 127.3: ArCH; 117.8, CH₂vinyl; 114.7, TyrArCH; 79.05, tBoc; 69.90, TyrOCH₂Ph; 66.51, GlyOCH₂Ph; 59.38, Tyrα; 53.46, CH; 49.28, Glyα; 35.44: coincident allyl carbon and Tyrβ; 28.20, Boc. Mass Spectrum (ISMS) m/z 545.1 (MH⁺), calculated for C₃₂H₄₅N₃O₅: 544

### Preparation of 60:

The amine 59 (930 mg, 1.7 mmol) was dissolved in ethyl acetate (15 mL) and 37% aq. formaldehyde solution added (1 mL). The solution was stirred vigorously at room temperature for 1 h (or until the reaction was complete) and then diluted with ether (100 mL) and washed in turn with aq. NaHCO₃, water (×3), brine and then dried (MgSO₄). Removal of solvent in vacuo left an approximately quantitative yield (950 mg) of the crude product 60 which was used in the next reaction or further purified by flash chromatography eluting with 10-15% ethyl acetate in light petroleum. TLC 33% EtOAc:light pet. Rf=0.56. The NMR spectra were quite broad in CDCl₃, amide rotamers were present in the approximate ratio 2:1. ¹H NMR (300 MHz, CDCl₃): δ 7.50-7.27, 10H, m's, Ar; 7.09, 2H, m, Ar; 6.90, 2H, d, J=8.5 Hz, Ar; 5.64, 1H, bm, vinyl CH; 5.19, 2H, s, OCH₂Bn; ˜5.1, 2H, m, vinyl CH₂; 5.05, 2H, s, OCH₂Bn; 4.59, 1H, bm, ring NCH₂N(a); 4.17, 1H, bm, ring NCH₂N(b); 4.06, 1H, bm, Tyrα; 3.70, 1H, d, J=17 Hz, Glyα(a); 3.42, 1H, bd, J=17 Hz, Glyα(b); 3.16, 1H, bm, TyrC′H(ring); 2.84, 2H, bm, Tyrβ; 2.31, 2H, m, allylCH₂; 1.38, ˜3H, bs, Boc minor rotamer; 1.19, ˜6H, s, Boc major rotamer. ¹³C NMR (75 MHz, CDCl₃): δ(peaks due to the carbamate rotamers are placed in parentheses, major rotamer first) 169.8 (ester); 157.2 (tyrosine O-ipso); (153.1, 152.8) carbamate; 137.2 (ipso); 135.4 (ipso); 134.2 (CH vinyl); 131.3 (ipso); 130.5, 128.5, 128.4, 128.3, 127.8, 127.4, 127.3, 126.9: ArCH; 117.5 (vinyl CH₂); 114.7 (2× TyrArCH); 79.52 (Boc tertiary); 69.93 (CH₂); 66.95 (CH₂); 66.46 (CH₂); 64.27 (CH); (59.65, 58.76) (CH); 51.60 (CH₂); 34.34 (CH₂); (32.20, 31.93) (CH₂); (27.93, 28.25) (Boc 3×CH₃). Mass Spectrum (ISMS) m/z 557.1 (MH⁺), calculated for C₃₄H₄₀N₂O₅: 556 fragments (OR 60): 501.1, (−tBu)

### Preparation of 61:

To 220 mg of 60 was added 60 mg of N-methylmorpholine-N-oxide (NMO), 40 mg of a 2.5% (by weight) solution of osmium tetroxide in t-butanol, 4 mLs of t-butanol and 0.5 mLs water. The mixture was stirred at room temperature until the reaction was complete (about 24 hours). 3 mLs of 10% NaHSO₃ was added, the solution stirred for 10 minutes, then neutralised with sodium bicarbonate, diluted with brine and extracted three times with ethyl acetate. The combined extracts were washed with brine and dried over magnesium sulfate. Removal of volatiles under reduced pressure gave the crude diol in good yield as an oil which could be used in the next reaction or purified if required by chromatography on silica gel eluting with ethyl acetate. Mass Spectrum (ISMS) m/z 591.3 (MH⁺), calculated for C₃₄H₄₂N₂O₇: 590

Oxidation of diol using Pb(OAc)₄: The diol (100 mg, 0.17 mmol) was dissolved in dry benzene (4 mL) and Pb(OAc)₄ (85 mg, moistened with acetic acid) was added. After 10 min stirring at room temperature the reaction was filtered, the solvent removed in vacuo and the residue purified by flash chromatography eluting with 25% EtOAc in light petroleum. Yield of the aldehyde 61 was 32% (30 mg). (No efforts to optimise the yield were made. Yield might be improved, for example, by partitioning the crude reaction mixture between aq. base and EtOAc to ensure none of the amine product was lost on filtration of the insoluble salts.) TLC 50% EtOAc in light pet. Rf=0.51. NMR analysis (NOESY experiment) indicated the 4,5-trans ring conformation (i.e. the 4(S) isomer). ¹H NMR (300 MHz, CDCl₃): δ 9.52, 1H, t, J=1.5 Hz, aldehyde; 7.50-7.25, 10H, m, ArH; 6.92, 2H, d, J=9 Hz, TyrAr; 5.15, 2H, s, OCH₂Ph; 5.05, 2H, s, OCH₂Ph; 4.65, 1H, bm, ringCH₂(i); 3.88, 1H, bm, Tyrα; 3.80, 1H, bm, ringCH₂(ii); 3.45, 1H, d, J=16 Hz, Glyα; 3.44, 1H, m, ringCH(βaldehyde); 3.28, bd, J=16 Hz, Glyα; 3.17, 1H, bm, Tyrβ; 2.80, 1H, dd, J=9.0, 13.5 Hz, Tyrβ; 2.51, 1H, J=6, 17 Hz, αaldehyde; 2.28, 1H, dd, J=17, 4.5 Hz, αaldehyde; 1.50, 9H, Boc. ¹³C NMR (75 MHz, CDCl₃), (rotamers): δ 200.5; 169.9; 157.5; 153.1; 136.9; 135.3; 130.5, 129.6, 128.6, 128.5, 128.4, 127.6, 127.4, 115.0: Ar; 80.21, tBoc; 69.92, OCH₂Ph; (67.08, 66.86) br, CH₂; 66.58, OCH₂Ph; (62.93, 62.56) br, CH; (61.35, 60.72) br, CH; 52.14, CH₂; 46.36, CH₂; (38.5, 37.27) br, CH₂; 28.38, Boc. Mass Spectrum (ISMS) m/z 559.1 (MH⁺), calculated for C₃₃H₃₈N₂O₆: 558

### Preparation of 62 and 63:

The aldehyde 61 (30 mg, 50 μmol) was dissolved in 1,2-dichloroethane (5 mL) and glycine methyl ester hydrochloride (50 mg) and NaBH(OAc)₃ (50 mg) added. The reaction was stirred at room temperature and was complete in a few minutes (<15 min). The reaction was diluted with ethyl acetate, and washed in turn with aq. NaHCO₃, water, brine and then dried (MgSO₄). Evaporation of the solvent left the crude product 62 as a clear oil: TLC 1:1 EtOAc:light pet. Rf=0.17. Mass Spectrum (ISMS) m/z 632.3 (M+H⁺), calculated for C₃₂H₄₅N₃O₅: 631 Analysis of the product or the reaction mixture after overnight standing revealed the formation of a new product with a mass spectrum corresponding to the target cyclised material 63 (MH⁺=524 Da). Thus the amine product 62 was not generally isolated but converted directly to 63. The spontaneous cyclisation was accelerated by the addition of base (i-Pr₂NEt). After removal of solvent by evaporation under reduced pressure and the product was purified by flash chromatography eluting with 10-20% EtOAc in light pet. TLC: 1:1 EtOAc:light pet. Rf=0.51. ¹H NMR (300 MHz, CD₃CN): δ 7.47-7.29, 5H, m, ArH; 7.12, 2H, m, Tyr; 6.92, 2H, m, Tyr; 5.07, 2H, s, OCH₂Ph; 4.35, 1H, d, J=5.4 Hz; AB_{q}, υa=4.05, υb=4.02, J_{AB}=17.4 Hz; 3.70-3.52, 6H, overlapped signals (includes: 3.65, 3H, s; 3.58, 1H, dd, J=11.2, 15.2 Hz); 3.49-3.32, 2H, br m's; 3.15, 1H, br dd, J=5.5, 15.5 Hz; 2.99, 1H, br dd, J=13.4, 14.9 Hz; 2.80, 1H, vbr m; 2.68, 1H, vbr m; 1.64, 1H, m; 1.46, 10H, s+m, Boc resonance obscures multiplet. ¹³C NMR (75 MHz, CD₃CN), rotamers, in approximate ratio 3:2, split some peaks and are recorded in parentheses: δ 173.3; 171.5; 158.8; 155.0, br; 138.9; 132.0; 129.9; 129.2; 129.0; 116.1; 80.84; 71.01; (70.87, 69.99); (68.12, 67.45); (65.47, 64.89); 55.76; 52.93; 51.45; 49.95; (39.00, 37.53); 31.87; 28.97 (Boc). Mass Spectrum (ISMS) m/z 524.3 (M+H⁺), calculated for C₂₉H₃₇N₃O₆: 523

Preparation of compounds 64 to 66:

The product 63 was hydrolysed with LiOH/H₂O/MeOH to the acid 64 (mass spectrum MH⁺=510) and then coupled (DMF/CH₂Cl₂/HBTU/DIEA) with phenethylamine using standard procedures and work-up to give 65. The imidazolidine ring of 65 was deprotected with a solution of acetic acid-methanol-water (˜1:1:1, stirred as a very dilute solution for several days then lyophilised) to give crude 66 as a white amorphous solid. Mass Spectrum (ISMS) m/z 601 (M+H⁺), calculated for C₃₅H₄₄N₄O₅: 600

### Example (B)

### Synthesis of a (4,5)-cis Imidazolidine Aldehyde by Oxidation of a Diol

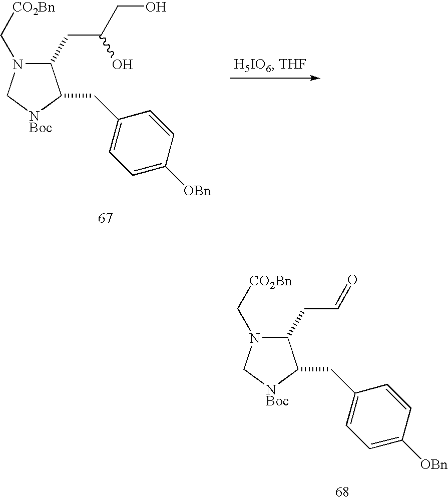

For the preparation of the 4,5-cis aldehyde 68 (in this case the 4(R) isomer) the diol 67 prepared from alkene 60 (as described above) (1 mmol) was dissolved in THF (10 mL) and H₅IO₆ (1 mmol) dissolved in THF (˜20 mL) was added and the reaction stirred at room temperature. A precipitate of iodic acid rapidly formed and the reaction was complete in <5 min. The THF solution was diluted with ether and washed in turn with 10% aq. Na₂CO₃, water, brine and then dried (MgSO₄). The product aldehyde 68 was formed in good yield and purity. Contact with acid should be minimised to prevent isomerisation to the trans aldehyde and/or decomposition, for example avoid chloroform as an NMR solvent unless recently made acid free. Yield was 60-80%. TLC: 50% EtOAc in light pet. Rf=−0.5. ¹H NMR (300 MHz, CD₃CN): 6 (peaks moderately broad; the Boc rotamers were not resolved although the Boc peak was asymmetric and very broad) 9.48, 1H, bm, aldehyde; 7.5-7.3, 10H, m, 2× Bn; 7.09, 2H, bd, J=7.5 Hz, Tyr Ar; 6.88, d, 8.2 Hz, Tyr Ar; 5.13, s, 2H, OCH₂Ph; 5.05, s, 2H, OCH₂Ph; 4.38, 1H, d, 6.0 Hz, NCH₂N(a); 4.22, 1H, m, Tyrα; 4.02, 1H, br, NCH₂N(b); 3.56, 1H, bd, J=17.2 Hz, Glyα(a); 3.48, 1H, m, TyrC′H, 3.29, 1H, bd, J=17.2 Hz, Glyα(b); 2.57-2.88, 4H, e, Tyrβ CH₂ and α-aldehyde CH₂; 2.22, s, H₂O; 1.48-1.08 (1.20 peak), 9H, vbr, Boc 3×CH₃. ¹³C NMR (75 MHz, CD₃CN): δ 201.9; 171.4; 158.7; 154.3; 139.0; 137.6; 132.6; 131.9, 129.92, 129.85, 129.6, 129.2, 128.9, 116.0: ArCH; 80.41 (Boc tert.); 70.99 (CH₂); 67.62 (br, CH₂); 67.44 (br, CH₂); 60.29 (2×CH, co-incident peaks determined by comparative intensity); 52.99 (br, CH₂); 43.58 (br, CH₂); 35.94 (br, CH₂); 28.78 (br, Boc 3×CH₃).

### Example (C)

### Synthesis of γ-Turn Mimetics I(i) for the Gly-Phe-Leu Sequence by the Short Method (which can be Used when R¹=Hydrogen)

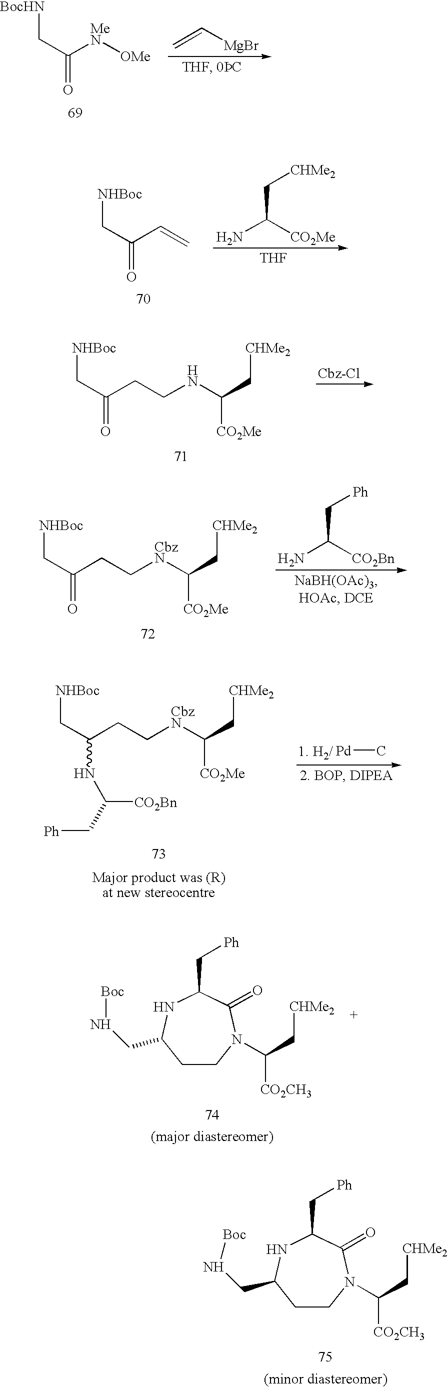

### Preparation of 69:

Boc-glycine was coupled with N,O-dimethyl hydroxylamine hydrochloride, I equivalent, in DMF/CH₂Cl₂ (1:5) using HBTU reagent (1 eq.) and DIEA (2 eq.) at room temperature. The CH₂Cl₂ was evaporated in vacuo and the residue partitioned between diethyl ether and aq. NaHCO₃. The aqueous layer was separated and the ether layer washed in turn with 1M HCl (×2), aq. NaHCO₃, brine, and then dried over MgSO₄. Filtration and removal of the solvent in vacuo left the product amide 69 as a viscous oil that slowly crystallised to a waxy solid and was further purified by chromatography on silica gel. Yield was >90%. ¹H NMR (300 MHz, CDCl₃): δ 5.3, 1H, bs, NH; 4.09, 2H, bd, αH₂; 3.72, 3H, s, OCH₃; 3.20, 3H, s, NCH₃; 1.46, 9H, s, Boc. ¹³C NMR (75 MHz, CDCl₃): δ 79.6; 61.4; 41.7; 32.4; 28.3.

### Preparation of 70:

A solution of 11.6 g (53 mmol) of Boc-glycine N,O-dimethylhydroxylamide in dry THF (70 mL) under nitrogen in a 250 mL round bottom flask was stirred and cooled in an ice bath. To this was added vinyl magnesium bromide in THF (˜120 mmol of a 1M solution) by syringe over 10 minutes. The solution was stirred for 2 h and then quenched by pouring into a mixture of crushed ice and 1M HCl which was then extracted with CH₂Cl₂ (×2). The organic extracts were washed with water/brine (×2), aq. NaHCO₃ and water/brine followed by drying over MgSO₄. Evaporation of the solvent left 9.6 g of a mobile oil (98% crude) which by NMR was −95% the ketone product 70. This material was used without further purification in the conjugate addition step. ¹H NMR (300 MHz, CDCl₃): δ 6.37, 2H, m (ABX, J_{ab}=2.5 Hz, J_{ax/bx}=9.0, 17.5 Hz), vinyl CH₂; 5.95, 1H, dd, J=2.5, 9.0 Hz, vinyl CH; 5.37, 1H, bs, NH; 4.26, 2H, d, J=4.6 Hz, glycyl αH₂; 1.46, 9H, s, Boc. ¹³C NMR (75 MHz, CDCl₃): δ 194.9 ketone; 155.8 carbamate; 133.6 vinyl; 129.6 vinyl; 79.8 tBoc; 48.32 Glyα; 28.28 Boc.

### Preparation of 71:

To a solution of 3.0 g (˜15 mmol) of crude 70 in THF (40 mL) was added 3.4 g of leucine methyl ester hydrochloride (˜1.2 eq) and 2.4 g (1.2 eq) of diisopropylethylamine. After 2 h the reaction was diluted with ether (200 mL) and extracted with cold 1M HCl (3×50 mL) (discard this ether layer). The aq. extracts were immediately neutralised with solid NaHCO₃ and this solution was then back extracted with ether, and the ether washed with water (×3) and finally brine and dried over MgSO₄. Evaporation of the solvent left ˜5.3 g of product 71 as an oil with very good purity, contaminated with a small amount of leucine methyl ester. Flash chromatography to separate the product was not very successful as the amine and amino ketone tended to co-elute. TLC EA/LP Rf=0.35. ¹H NMR (300 MHz, CDCl₃): δ 5.36, 1H, bm, NHBoc; 4.03, 2H, d, J=5 Hz, Glyα; 3.72, 3H, s, OCH₃; 3.26, 1H, t, J=7.5 Hz, Leuα; 2.93, 1H, dt, J=12, 6 Hz; 2.72, 1H, dt, J=12, 6 Hz; 2.50, 2H, m; 2.0, 1H, bs, NH; 1.69, 1H, m, Leuγ; 1.45, 11H, m, Boc(9H) and Leuβ(2H); 0.90, 6H, m, Leuδ. ¹³C NMR (75 MHz, CDCl₃): δ 205.1; 176.1; 155.5; 79.8 tBoc; 60.04; 51.64; 50.53; 42.63; 42.57; 40.55; 28.26 Boc; 24.81; 22.63; 22.17. Mass Spectrum (ISMS) m/z 331.4 (M+H⁺), calculated for C₁₆H₃₀N₂O₅: 330; fragments (OR 60): 275.2 (-tBu).

### Preparation of 72:

The amine 71 was protected as the benzyl carbamate by standard procedures as follows: the crude amine product 71 (1.68 g, ˜5 mmol) was dissolved in ethyl acetate (30 mL) to which was added a solution of KHCO₃ (1.2 g) in water (15 mL). This mixture was vigorously stirred and cooled in an ice bath and to it was added benzyl chloroformate (780 uL of a 95% solution, 5.2 mmol) dropwise over 5 min. The reaction was stirred for a further 15 min then allowed to warm to room temperature with stirring for an additional 2 h. After this time the mixture was diluted with ether (100 mL), the aqueous layer seperated, and the organic layer washed with 1M HCl, aq. NaHCO₃, brine and then dried over MgSO₄. Evaporation of the solvent left ˜2.6 g crude oil which was purified by flash chromatography eluting with 25% EtOAc in light pet; combination of the main fractions gave a yield of 86% (2.02 g) of 72. TLC EA:2LP Rf=0.56. NMR signals split due to amide rotamers (˜1:1) are placed in parentheses where possible. ¹H NMR (300 MHz, CDCl₃): δ 7.40-7.23, 5H, Ar; 5.28-5.02, 3H, m's, CH₂Ph+NH; (4.64, m, 4.43, m) 1H; (3.98, bs, 3.88, bs) 2H, 3.72-3.51, 4H, includes (3.67, s, 3.55, s) OCH₃+1H, 3.45, 1H, m; 2.78, 2H, m; 1.75, 2H, m; 1.53, 1H, m; 1.43, 9H, s, Boc; 0.91, 6H, m, Leuδ CH₃×2. ¹³C NMR (75 MHz, CDCl₃): δ (204.9, 204.5) ketone; (172.5, 172.3) ester; (156.1, 155.8) carbamate; 155.6, carbamate; (136.2, 136.0) ipso; 128.5, 128.2, 128.1, 128.0: ArCH; 79.80, tBoc; 67.48; (58.50, 58.32); 52.12; 50.30; (41.37, 39.87, 39.78, 38.87, 38.60, 37.98) 3C, 28.23, Boc; (24.83, 24.67); 23.09; (21.46, 21.39). Mass Spectrum (ISMS) m/z 465.3 (MH⁺), calculated for C₂₄H₃₆N₂O₇: 464; fragments (OR 70): 409.2, (-tBu); 365.2, (-Boc).

Preparation of amines 73:

To a solution of 72 (700 mg, 1.5 mmol) in 15 mL of 1,2-dichloroethane was added phenylalanine benzyl ester p-toluene sulfonate (900 mg, 2.1 mmol) and sodium triacetoxy borohydride (850 mg, 4.0 mmol). The mixture was stirred at room temperature for 24 h and then the solvent removed under vacuum and the residue partitioned between ethyl acetate and aq. NaHCO₃, the aqueous layer separated, and the organic layer washed with water then brine and then dried over MgSO₄. Evaporation of the solvent left 1.2 g crude oil which was purified by flash chromatography eluting with 25-40% EtOAc in light petroleum ether to give a yield of 76% (800 mg) of the product (a clear oil). The product diastereomers 73 were not seperable under these chromatography conditions. TLC 40% EA in LP Rf=0.48. ¹H NMR (300 MHz, CD₃CN): δ (not very informative due to the presence of rotamers/diastereomers) 7.45-7.05 aromatic protons; (5.46 m, 5.31 m)˜½H, 5.15-5.00, ˜4H, m, OCH₂Ph; 4.95, ˜¼H, m; (4.51, m, 4.37, m): 1H, 3.85-3.10, ˜5H, e (including 3.63, s, 3.58, s: 3H, OCH₃); 3.10-2.70, 5H, e; 2.45 broad water peak; 1.80-1.45, 5H, m's; 1.40, 9H, s, Boc; 0.90, 6H, bs, Leuδ. ¹³C NMR (75 MHz, CD₃CN): 6 (signals are grouped in parentheses where they can be reasonably assigned to equivalent carbons in the different diastereomers/rotamers) (175.6, 175.4 (br)); 173.6; 157.4, 157.2 (br); (139.0, 139.2, 138.5, 138.3, 137.3) 3×ipso; 130.8, 130.7 129.9 129.71, 129.66, 129.3, 129.0, 128.0: Ar CH; (79.87, 79.62) Boc tertiary; 68.22 (CH₂, OBn); 67.75 (CH₂, OBn); (61.67, 61.55) (CH); 59.39 (CH); (56.51, 55.82, 55.61) (CH); 53.11 (OCH₃); (45.56, 45.16, 44.73, 44.61, 44.43, 44.24, 43.42, 43.04) (2×CH₂); (40.77, 40.15, 40.03, 39.42, 39.27) (2×CH₂); (39.66, 32.60, 32.45, 31.44) (CH₂); 29.04 (CH₃ Boc); 29.93 (CH); 23.88 (CH₂); 22.36 (CH₂). Mass Spectrum (ISMS) m/z 704.4 (M+H⁺), calculated for C₄₀H₅₃N₃O₈: 703

### Preparation of 74 and 75:

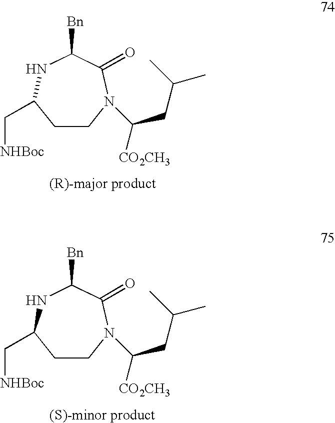

The mixture of epimeric amines 73 (260 mg, 0.4 mmol) was dissolved in methanol (20 mL) and 10% palladium on carbon added (100 mg). The solution was hydrogenated (40 psi H₂) at room temperature for 3 h to give the deprotected amino acid (MH⁺=480 Da). After filtration, the solvent was removed and the residue (170 mg) was dissolved in DMF (5 mL) and diluted with CH₂Cl₂ (50 mL). To this solution was added HBTU (180 mg, 0.48 mmol) and DIEA (150 mg, 1.2 mmol). After stirring for 10 min at room temperature the solution was diluted with aq. NaHCO₃, the aqueous layer separated, and the organic layer washed with water (×3) then brine and then dried over MgSO₄. Evaporation of the solvent left an oil which was purified by flash chromatography eluting with 20-40% EtOAc in light petroleum ether. The product diastereomers were just separable under these conditions, with the minor diastereomer 75 eluting first to give a yield of 18% (30 mg) followed by the major diastereomer 74 in 50% (85 mg) yield. TLC EA:LP 1:1 Rf=0.43, 0.29. ¹H NMR (300 MHz, CD₃CN): δ Isomer 75: 7.29, 4H, m, ArH; 7.22, 1H, m, ArH; 5.17, 1H, dd, J=6.5, 8.4 Hz; 5.08, 1H, m; 3.65, 3H, s, OCH₃; 3.61, 1H, dd, J=11.4, 15.6 Hz; 3.27, 1H, ddd, J=1.5, 5.7, 15.9 Hz; 3.12, 1H, dd, J=4.5, 14.3 Hz; 2.98, 1H, bm; 2.72, 1H, m; 2.64, 1H, dd, J=9.9, 14.3 Hz; 2.57, 1H, bm; (2.17, H₂O); 1.68, 3H, m; 1.60, 1H, m, Leuγ; 1.36, 9H, s, Boc; 1.16, 1H, m; 0.95, 3H, d, J=6.4 Hz, Leuδ; 0.93, 3H, d, J=6.6 Hz. Isomer 74: 7.29, 4H, m, ArH; 7.22, 1H, m, ArH; 5.11, 1H, dd, J=5.6, 9.4 Hz; 4.29, 1H, br, NHBoc; 3.81, 1H, dd, J=4.6, 9.8 Hz; 3.65, 3H, s, OCH₃; 3.59, 1H, dd, J=10.8, 15.2 Hz; 3.19, 1H, dd, J=5.5, 15.2 Hz; 3.13, 1H, dd, J=4.5, 13.8 Hz; 2.94, 2H, m's; 2.71, 1H, m; 2.64, 1H, dd, J=10.3, 13.3 Hz; (2.17, H₂O); 1.76, 1H, m; 1.69, 2H, m; 1.57, 2H, m; 1.36, 9H, s, Boc; 0.93, 6H, d, J=6.5 Hz. ¹³C NMR (75 MHz, CDCl₃): δ Isomer 75 (5S): 175.2; 172.5; 155.9; 138.9; 129.3; 128.5; 126.4; 79.2; 60.91; 60.62; 55.65; 52.19; 45.70; 43.98; 38.12; 37.99; 33.46; 28.30, Boc; 25.01; 23.10; 21.93. Isomer 74 (5R): 175.1; 172.5; 155.7; 139.3; 129.3; 128.7; 126.8; 78.9; 56.01; 55.80; 53.05; 52.14; 42.07; 40.70; 38.01; 37.98; 31.51; 28.26, Boc; 25.03; 23.11 21.74. Mass Spectrum (ISMS) m/z 462.3 (MH⁺), calculated for C₃₂H₄₅N₃O₅: 461 fragments (OR 70): 406.2 (-tBu).

### Example (D)

### Selective Synthesis of the 3(S), 5(S) Diastereomer 75 by the Short Method

The 3(S)5(S) diastereomer, the minor product formed as described above, can be selectively synthesised by the use of an intramolecular reductive amination-cyclisation as described below:

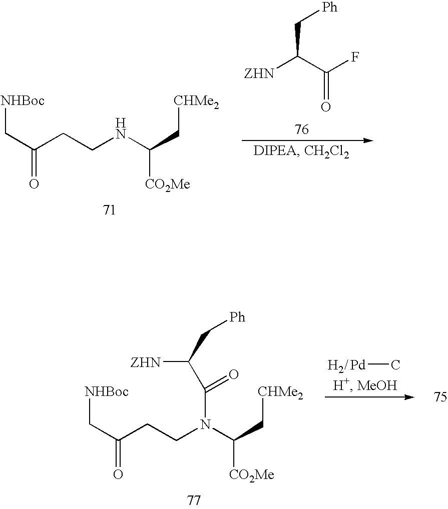

Preparation of acyl fluoride 76: Z-phenylalanine acid fluoride was prepared by general literature methods(Carpino, Sadat-Aalaee et al. 1990; Wenschuh, Beyermann et al. 1994) as follows: 1.1 equivalents of diethylaminosulfurtrifluoride (DAST) were added to ZPheOH in dry dichloromethane solution under nitrogen at 0° C. After stirring for 15 min the reaction was worked up by pouring onto iced water and separating the organic layer, washing once with cold water and then drying over MgSO₄. The product was purified by precipitation from ether/petroleum ether and dried in vacuo. ¹H NMR (300 MHz, CDCl₃): δ 7.36, 8H, m's; 7.28, 2H, m; 5.30, 1H, bd; J=7.5 Hz, NH; 5.13, 2H, s, OCH₂Ph; 4.85, 1H, m, αH; 3.20, 2H, m, βH₂. ¹³C NMR (75 MHz, CDCl₃): δ 161.8, d, ¹J_{CF}=370 Hz; 155.5; 135.7; 134.2; 129.1; 129.0; 128.5; 128.3; 128.1; 127.7; 67.36; 53.50, d, ²J_{CF}=59 Hz; 36.70.

### Preparation of 77:

To the amine 71 (2.7 g, 8.2 mmol) dissolved in CH₂Cl₂ (40 mL) was added Z-phenylalanine acid fluoride 76 (prepared as described above) (3.0 g, 10 mmol) and DIEA (1.3 g, 10 mmol) and the solution stirred at room temperature under nitrogen for 30 h. The solvent was then evaporated in vacuo and the residue dissolved in ether and extracted in turn with 1M HCl (×2), 10% aq. Na₂CO₃ (×2), then brine and then dried over MgSO₄. The solution was filtered and the solvent removed in vacuo. The resulting oil was purified by flash chromatography eluting with 20-40% ethyl acetate in light petroleum ether for a yield of about 80% of the target 77 as a clear oil. TLC 40% EA:LP Rf=0.40. ¹H NMR (300 MHz, CDCl₃): δ 7.41-7.13, 10H, Ar; 5.48, 1H, bd, J=9.2 Hz, NHCbz; 5.19, 1H, bm, NHBoc; 5.09, 2H, s, OCH₂Ph; 4.76, 1H, dt, J=6.4, 8.9 Hz, Pheα; 4.38, 1H, dd, J=5.2, 9.3 Hz, Leuα; 3.92, 2H, d, J=4.5 Hz, Glyα; 3.60, 3H, s, OCH₃; 3.54, 1H, m; 3.38, 1H, m; 3.08, 1H, dd, J=8.4, 13.3 Hz; 2.93, 1H, dd, J=6.1, 13.1 Hz; 2.65, 2H, m; 2.80, 1H, m; 2.64, 1H, m; 1.46, 9H, s, Boc; ˜1.38, 1H, m; 0.90, 6H, 2×d, J=6.6, 6.5, Leuδ. ¹³C NMR (75 MHz, CDCl₃) amide rotamers (˜5:1): only the major peak of rotamer peak pairs is reported: δ 204.1; 172.1; 171.4; 156.7; 155.6; 136.2; 135.8; 129.4-127.1: ArCH; 79.8; 66.82; 58.15; 52.25; 52.05; 50.28; 41.32; 39.58 (2 coincident signals as determined by relative intensity, shift and the presence of both minor rotamer peaks); 37.82; 28.23, Boc; 24.67; 23.08; 21.67. Mass Spectrum (ISMS) m/z 612.3 (M+H⁺), calculated for C₃₃H₄₅N₃O₈: 611; fragments: (OR 60): 556.3 (-tBu); 512.3 (-Boc).

Selective Preparation of 75 from 77:

The ketone 77 (μmol) was dissolved in 0.1M methanolic HCl (30 mL) and 10% palladium on activated carbon (200 mg) was added. The solution was hydrogenated at 30 psi H₂ (room temperature) for 8 h and then diluted with aq. NaHCO₃ and extracted with ethyl acetate. The organic layer was washed with water (×2) and then brine then dried over MgSO₄. Filtration and removal of solvent in vacuo left the crude product 75 in good yield and purity. Analysis of the crude product by NMR and by TLC did not reveal any of diastereomer 74. The reaction was estimated to be >95% stereoselective.

### Example (E)

### Synthesis of a Biologically Active γ-Turn Mimetic for the Arg-Gly-Asp Sequence

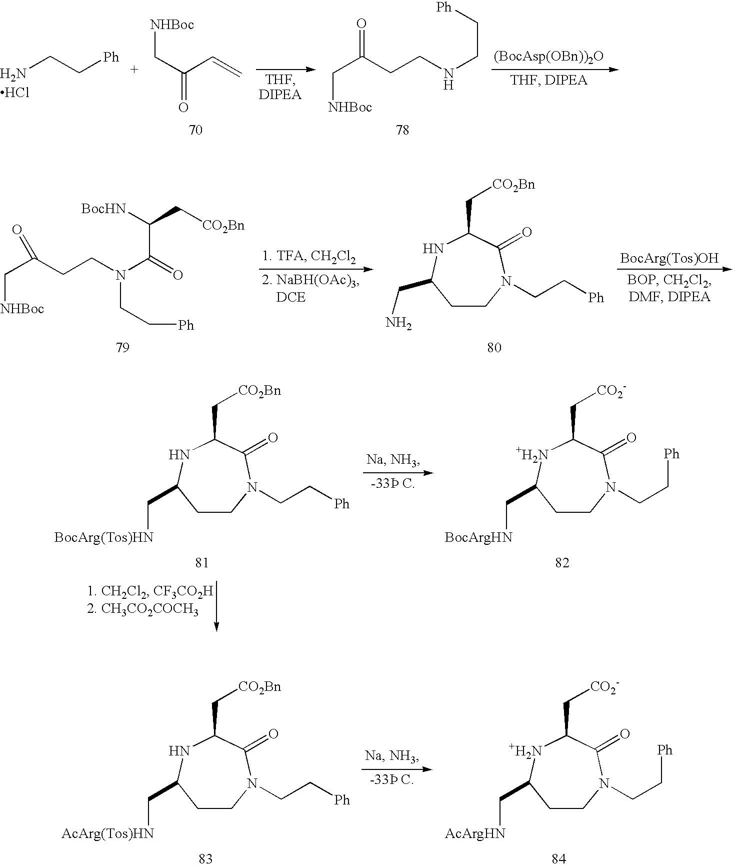

Preparation of 78: The α,β-unsaturated ketone 70 (1.0 g, 5.4 mmol, prepared as previously described) was reacted with phenethylamine hydrochloride (1.07 g, 6.8 mmol) and DIEA in THF by the method previously described for the preparation of 71. The crude product 78 was used without further purification for the next reaction. Mass Spectrum (ISMS) m/z 307.2 (MH⁺), calculated for C₁₇H₂₆N₂O₃: 306; fragments (OR 60): 250.9 (-tBu).

### Preparation of 79:

To a stirred solution of Boc-aspartic acid β-benzyl ester (3.23 g, 10 mmol) in CH₂Cl₂ (10 mL) was added dicyclohexylcarbodiimide (10 mL of 0.5M solution in CH₂Cl₂) at room temperature. A copious precipitate of dicyclohexylurea soom formed; after 10 min the solution was filtered, and the solvent removed in vacuo. The residual oil was added to a solution of crude 78 (1.3 g) in THF, followed by DIEA (645 mg, 5 mmol), and the solution stirred for 4 h. The reaction mixture was diluted with ether/ethyl acetate and washed with 1M HCl, aq. NaHCO₃, water, brine and dried over MgSO₄. The crude product was purified by flash chromatography eluting with 30-50% ethyl ether in petroleum ether to give a reasonable yield of 79 (estimated as 80% based on 78) as a clear oil. ¹H NMR (300 MHz, CDCl₃, amide rotamers present): δ 7.38-7.16, 10H, m, Ar; 5.37, 1H, bd, J=9 Hz, AspNHBoc (minor rotamer 5.33, J=10 Hz); 5.25, m, 1H (Gly NH); 5.10, 2H, m, OCH₂Ph; 4.89, 1H, m; 3.93, 2H, d, J=4.4 Hz, Glyα; 3.67-3.53, 3H, m's; 3.47, 1H, m; 2.95-2.52, 6H, m's (including 2.88, 2H, m; 2.63, 2H, ABX, J=15.8, 7.3, 5.8 Hz, βH₂Asp); 1.44, 18H, multiple singlets, 2×Boc. ¹³C NMR (75 MHz, CDCl₃): δ (major rotamer only) 204.7; 171.0; 170.3; 155.6; 154.8; 137.7; 135.5; 128.9, 128.6, 128.5, 128.2, 126.6: ArCH; 80.06; 79.73 (2×tBoc); 66.57; 50.55; 50.33; 46.99; 42.24; 37.69 (2 signals); 35.50; 28.22 (2×Boc). Mass Spectrum (ISMS) m/z 612.3 (MH⁺), calculated for C₃₃H₄₅N₃O₈: 611 fragments (OR 60): 556.1 (-tBu); 512.1 (-Boc).

### Preparation of 80:

The ketone 79 (390 mg, 0.64 mmol) in CH₂Cl₂ (2 mL) was treated with trifluoroacetic acid (2 mL) and the solution stirred for 30 min at room temperature. The volatiles were then removed in vacuo and CH₂Cl₂ (3 mL) added and removed in vacuo (×2). The residual oil was dissolved in 1,2-dichloroethane (5 mL) and NaBH(OAc)₃ (270 mg, 1.3 mmol) added. The mixture was stirred for 20 min then the solvent removed and the residue dissolved in ethyl acetate and washed with aq. Na₂CO₃ and then brine and then dried over MgSO₄. The crude product 80 (after solvent removal 210 mg, 84%) was of good purity by MS and NMR, with only one diastereomer observed (>95% diastereoselectivity). ¹H NMR (300 MHz, CDCl₃): δ 7.39-7.10, 10H, m, Ar; {5.20, 5.16, 5.14, 5.10}, 2H, ABq, J=12.5 Hz) OCH₂Ph; 3.86, 1H, t, J=6.3; 3.76-3.43, 3H, m's; 3.14, 1H, bdd, J=15, 5 Hz; 2.98-2.76, 5H, e; 2.70, 1H, dd, J=7.4, 16 Hz; 2.46, 1H, m; 1.64, 1H, bm; 1.06, 1H bm. ¹³C NMR (75 MHz, CDCl₃): δ 173.9; 172.0; 138.9; 135.9; 128.7, 128.4, 128.0, 126.3: Ar; 66.16; 60.49; 56.55; 51.24; 48.39; 45.14; 38.05; 34.15; 33.01. Mass Spectrum (ISMS) m/z 396.2 (MH⁺), calculated for C₂₃H₂₉N₃O₃: 395.

### Preparation of 81:

The crude amine product 80 (140 mg, ˜0.35 mmol) was coupled with BocArg(Tos)OH (182 mg, 1.2 eq) using the BOP reagent (188 mg) and DIEA (55 mg) in DMF/CH₂Cl₂ (5 mL). The CH₂Cl₂ was evaporated in vacuo and the residue partitioned between diethyl ether/ethyl acetate and aq. NaHCO₃. The aqueous layer was separated and the organic layer washed in turn with 1M HCl (×2), water (×2), aq. NaHCO₃, brine, and then dried over MgSO₄. Filtration and removal of the solvent in vacuo left the crude product amide 81 which was purified by flash chromatography eluting with 5-10% ethanol in ethyl acetate (yield 260 mg, 90%). TLC 10% EtOH in EtOAc Rf=0.38. ¹H NMR (300 MHz, CD₃OD): δ 7.74, 2H, d, J=7 Hz; 7.4-7.15, 12H, m's; 5.15, 2H abq, J=11 Hz, OBn; 4.26, 1H, m; 4.03, 1H, m; 3.73, 2H, m; 3.48-3.07, 7H, e; 3.07, 1H, m; 2.92-2.73, 3H, m's; 1.92, 1H, m; 1.73, 1H, m; 1.66-1.45, 4H, e; 1.42, 9H, s, Boc. ¹³C NMR (75 MHz, CD₃OD): δ 176.1; 172.5; 172.0 (br); 158.8; 158.1; 143.7; 142.2; 140.3; 137.5; 130.4; 130.1; 129.72; 129.68; 129.4; 128.4; 127.6; 127.3; 127.2; 80.92 (t); 67.75 (CH₂); 62.55 (CH); 57.27 (CH); 56.00 (CH); 52.55 (CH₂); 48.74 (CH₂); 44.42 (CH₂); 41.22 (br, CH₂); 37.00 (CH₂); 35.10 (CH₂); 32.41 (CH₂); 30.15 (CH₂); 28.87 (Boc CH₃); 27.24 (br, CH₂); 21.57 (CH₃). Mass Spectrum (ISMS) m/z 806.4 (MH⁺), calculated for C₄₁H₅₅N₇O₈S: 805

### Preparation of 82:

The amine 81 (50 mg, 0.06 mmol) in THF (0.6 mL) was cooled in a dry ice acetone bath and ammonia gas added until ˜30 mL of ammonia had condensed. Small pieces of sodium metal (3-6 mg) were added until the blue colour persisted. The reaction was quenched by the addition of ammonium carbonate (25 mg), the dry ice bath removed and the solvent allowed to evaporate at room temperature. The residue (which gave a crude mass spectrum with the product mass as the only significant peak) was purified by reversed phase HPLC (Vydac C18) eluting with 85% solvent A (=0.1% CF₃COOH in H₂O):15% solvent B (=0.1% CF₃COOH and ˜10% H₂O in CH₃CN) for 2 minutes followed by a 2%/min gradient. Only one product diastereomer was observed in the HPLC traces. Mass Spectrum (ISMS) m/z 562.3 (M+H⁺), calculated for C₂₇H₄₃N₇O₆: Preparation of 83: The amine 81 was dissolved in CH₂Cl₂/CF₃CO₂H (2 mL, 1:1) and stirred at room temperature for 30 minutes after which the Boc group had been removed. 10 mL of CH₂Cl₂ was then added and the volatiles removed in vacuo (repeat once). The residue was again dissolved in CH₂Cl₂ and acetic anhydride (2 eq.) added along with diisopropylethylamine (DIEA, 5 eq.), and the reaction stirred at room temperature for 2 h. The volatiles were removed in vacuo and the residue dissolved in ethyl acetate and washed with aq. NaHCO₃ then brine and then dried over MgSO₄. Filtration and removal of the solvent in vacuo left the crude product 83 as an oil in reasonable purity. The ¹H NMR was badly broadened in common solvents at room temperature.

¹³C NMR (75 MHz, CDCl₃): δ 173.7; 172.4; 171.9; 171.0; 157.0; 142.1; 140.4; 138.8; 135.8; 129.2, 128.7, 128.4, 128.1, 128.0, 126.3, 125.8: ArCH; 66.22, OCH₂Ph; 60.08, CH; 56.09, CH; 52.94, br, CH; 51.06, CH₂; 48.21, CH₂; 44.31, CH₂; 40.13, br, CH₂; 37.79, CH₂; 34.16, CH₂; 32.97, CH₂; (29.59, 29.50) 1C, br, CH₂; 25.64, br, CH₂; 22.91, CH₃; 21.32, CH₃. Mass Spectrum (ISMS) m/z 748.2 (MH⁺), calculated for C₃₇H₄₉N₇O₇S: 747

### Preparation of 84:

Compound 84 was prepared from 83 by dissolving metal reduction as described for the preparation of 82 above. Purification was carried out by HPLC under the same conditions as for 82.

### Testing of Arg-Glv-Asp Mimetics 82 and 84 for Inhibition of Platelet Aggregation in Human Platelet Rich Plasma (PRP).

The peptide sequence arginine-glycine-aspartic acid (RGD) is important to the binding of proteins to certain integrin receptors, such as the GP_{IIb-IIIa} receptor found on the surface of platelets. Several cyclic peptides having the RGD sequence have been found to antagonise the binding of plasma proteins to the GP_{IIb-IIIa} receptor, thereby inhibiting blood clotting. GP_{IIb-IIIa} antagonists have therapeutic potential as anti-thrombotics, there are several in early clinical trials(Humphries, Doyle et al. 1994). Mimetics based on γ-turn structures centred on the Asp residue have been successful, this structure was chosen to test the compounds of the invention.

Solutions of the compounds to be tested were made up in water. Platelet aggregation induced by adenosinediphosphate (ADP, 10 μM) in human PRP was measured by the decrease in light scattering on aggregation, measured with a platelet aggregometer. The tetra-peptide Ac-Arg-Gly-Asp-Ser-NH₂ was used as a positive control (Callahan, Bean et al. 1992). Compounds 82 and 84 were both found to inhibit platelet aggregation in a dose dependent manner, and both exhibited stronger inhibition than the control peptide. Compound 84 was the strongest, having inhibitory activity approximately five times more potent than Ac-Arg-Gly-Asp-Ser-NH₂ under the conditions of the test.

### Example (F)

### Synthesis of Fully Substituted γ-Turn Mimetics for the Phe-Leu-Ala Sequence in Both the 4(R) and 4(S) Configurations

The synthesis up to the final common intermediate for the 4(R) and 4(S) diastereomers, the aldehyde 93, is summarised below:

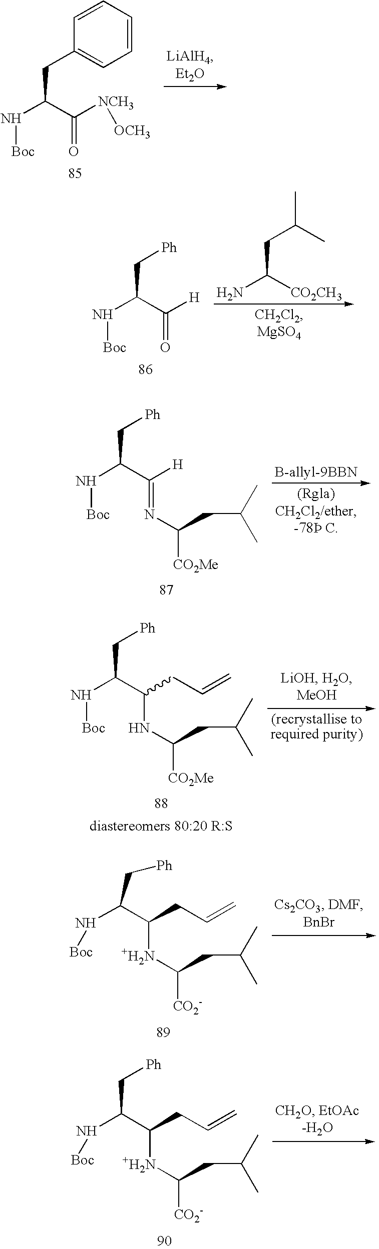

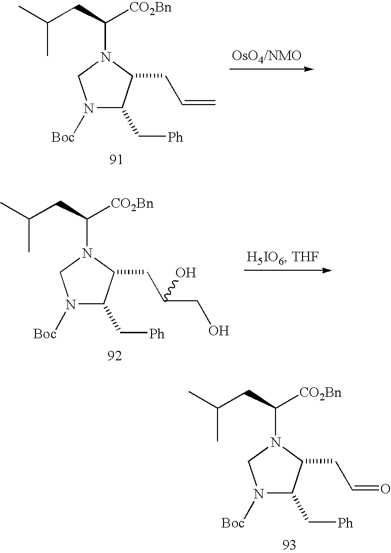

Bocphenylalanine N,O-dimethylhydroxylamide 85 was synthesised by the general solution phase coupling procedure as previously described from Boc-phenylalanine and N,O-dimethyl hydroxylamine hydrochloride. Yield: ˜quantitative. Purification: on a short silica column eluting with ether. ¹H NMR (300 MHz, CDCl₃): δ 7.33-7.12, 5H, m, Ar; 5.20, 1H, bd, J=7 Hz, NH; 4.95, 1H, bm, Pheα; 3.66, 3H, s, OCH₃; 3.17, 3H, s, NCH₃; 3.06, 1H, dd, J=6, 13.5 Hz, Pheβ; 2.88, 1H, dd, J=7.5, 13.5 Hz; 1.40, 9H, s, Boc. ¹³C NMR (75 MHz, CDCl₃): δ 172.2; 155.1; 136.5; 129.4; 128.2; 126.7; 79.5; 61.4; 51.4; 38.8, Pheβ; 32.0; 28.2, Boc.

The amide 85 was reduced to Bocphenylalanine aldehyde 86 by the method of Fehrentz and Castro (Fehrentz and Castro 1983). Briefly: amide (2 mmol) dissolved in dry ether (20 mL) and cooled and in an ice bath under nitrogen, then LiAlH₄ (95 mg, 2.5 mmol) added and stirring continued 15 min. Then KHSO₄ (477 mg, 3.5 mmol) in 10 mL water added and then 150 mL ether and wash with 1M HCl (cold) (×3), aq. NaHCO₃, brine, and dried over MgSO₄. Removal of the solvent left the solid aldehyde in ˜90% crude yield containing some of the overreduced alcohol as the only significant impurity. TLC EtOAc:light pet. Rf=0.5. ¹H NMR (300 MHz, CDCl₃): δ 9.63, 1H, s, aldehyde; 7.37-7.13, 5H, m, Ar; 5.07, 1H, bs, NH; 4.43, 1H, m, Pheα; 3.11, 2H, d(AB) Pheβ; 1.43, 9H, s, Boc. ¹³C NMR (75 MHz, CDCl₃): δ 199.4, aldehyde; 155.3, carbamate; 135.7, ipso; 129.3, 128.7, 127.1: ArCH; 80.2, tBoc; 60.8, Pheα; 35.5, Pheβ; 28.2, Boc

Methyl leucinate hydrochloride (0.80 g, 4.4 mmol) was neutralised with 10% aq. Na₂CO₃ solution (25 mL), and the solution was mixed with brine (25 mL) and extracted with CH₂Cl₂ (3×20 mL). The organic extracts were dried over MgSO₄ and most of the solvent removed under vacuum (˜2 mL residue). This solution of methyl leucinate was added to Boc phenylalanine aldehyde 86 (1.1 g, 4.4 mmol) in CH₂Cl₂ (5 mL), the stirred solution soon became turbid due to the separation of water, dried MgSO₄ (500 mg) was added and the solution cleared. After 30 min the solution was filtered into a dried flask under nitrogen. NMR analysis showed that all the aldehyde had been converted to the imine 87 and that significant racemisation had not taken place. The imine was used without further purification for the allylation reaction. ¹H NMR (300 MHz, CDCl₃): δ 7.61, 1H, d, J=1.3 Hz, imine; 7.32-7.14, 5H, m, Ar; 5.69, 1H, bd, J=4.5 Hz, NH; 4.49, 1H, m, Pheα; 3.85, 1H, dd, J=5.5, 8.5 Hz, Leuα; 3.69, 3H, s, OCH₃; 3.20, 1H, dd, J=5.0, 14.5 Hz, Pheβ; 2.96, 1H, dd, J=8.0, 13.5 Hz; Pheβ; 1.63, 1H, m; 1.46, 9H, s, Boc; 1.42, 1H, m; 1.30, 1H, m; 0.88, 3H, d, J=6.5 Hz, Leuδ; 0.80, 3H, d, J=6.5 Hz, Leuδ. ¹³C NMR (75 MHz, CDCl₃): δ 171.7, ester; 164.3, CH, imine; 154.6, carbamate; 136.1, ipso; 128.9, 127.7, 126.0: ArCH; 78.56, tBoc; 69.51; 54.08; 51.32; 41.02, CH₂; 38.04, CH₂; 27.73, Boc; 22.35; 22.48; 20.63.

B-allyl-9-borabicyclononane Rg1a can be synthesised from B-methoxy-9-borabicyclononane (synthesised in turn from the methanolysis of 9-BBN(Kramer and Brown 1974)) by the method of Kramer (Kramer and Brown 1977). Alternatively the following one-pot synthesis from 9-BBN was used: a suspension of 9-BBN (crystalline dimer, 8.97 g, 73.5 mmol) in anhydrous ether (75 mL) was stirred under nitrogen and cooled to 0° C. Methanol (3.3 mL, 81 mmol) was slowly added by syringe (gas evolved), and vigorous stirring continued for ˜3 h (9-BBN gradually dissolves, gas evolution ceases). Allylmagnesium bromide in ether (81 mL of a 1.0M solution) was slowly added to the solution (still cooled to 0° C.); (a thick grey ppt. forms, stirring may be difficult). Stirring was continued for 1 h then the solution was allowed to warm to room temperature and the ether was pumped off under moderate vacuum (−300−>20 mbar). The residue was re-suspended in anhydrous hexane (100 mL) and then stirring stopped to allow the magnesium salts to settle out. The solution was estimated by reaction with a known amount of methylphenylketone in ether (found to be ˜0.57M, equal to 78% yield). The clear solution of B-allyl-9-BBN was used directly for allylation of the imines. (This procedure was adapted from one described by Rachlera and Brown (Racherla, Liao et al. 1992)). The imine 87 (˜23 mmol) was dissolved in dry diethylether (100 mL) under nitrogen and the stirred solution cooled to −78° C. B-allyl-9-BBN (47.5 mL of ˜0.57M solution in hexane, ˜27 mmol) was added and the solution stirred for 1 h and then allowed to warm to room temperature with stirring for an additional 1 h. Glacial acetic acid (1.5 mL) was added and the ether was removed in vacuo. The residue was dissolved in acetonitrile (100 mL) and more glacial acetic acid (5 mL) added. The solution was then refluxed until all of the borane adduct had been converted to the amine (˜24 h, monitored by TLC: Rf adduct>Rf amine=0.32 in 1:5 EtOAc:light pet.). The acetonitrile was removed in vacuo and the residue partitioned between ether/light petroleum and 10% aq. Na₂CO₃. The organic layer was washed again with 10% aq. Na₂CO₃ and then extracted with a solution of 25% methanol in 0.5M HCl (three times), the organic layer containing the neutral reaction products (˜6 g) was discarded. The aq. acid extracts were immediately neutralised with solid NaHCO₃ and then extracted with ether. The ether solution was washed with water then brine and then dried over MgSO₄. Evaporation of the solvent left the amine products (5.9 g) which were further purified by flash chromatography eluting with 7.5-15% ethyl acetate in light petroleum for a yield of 50+% of the amines 88 based on the crude aldehyde 86 used in the imine formation. Some separation of the diastereomers was observed in the chromatography, but they were not well resolved. Alternatively the crude amines were hydrolysed to the amino acid as described below and purified by recrystallisation. ¹H NMR (300 MHz, CDCl₃), major diastereomer: δ 7.32-7.13, 5H, m, Ar; 5.84, 1H, m, vinylCH; 5.11, 2H, m, vinylCH₂; 5.00, 1H, d, J=8 Hz, NHBoc; 3.88, 1H, m, Pheα; 3.66, 3H, s, OCH₃; 3.40, 1H, t, J=7 Hz, Leuα; 2.87, 1H, dd, J=5, 13 Hz, Pheβ; 2.69, 2H, m's: Pheβ+CH(homoallyl); 2.23, 2H, m, allyl; 1.7, 1H, b, NH(amine); (1.65, 1H, m; 1.47, 2H, m) Leuβ+γ; 1.33, 9H, s, Boc; 0.90, 6H, t(2 doublets) J=7, 7 Hz, Leuδ. ¹³C NMR (75 MHz, CDCl₃), major isomer: δ 176.1; 155.4; 138.6, ipso; 135.2, CH vinyl; 129.2, 128.2, 126.1: CHAr; 117.4, CH₂ vinyl; 78.8, tBoc; 58.94; 58.56; 54.10; 51.71; 42.87; 36.52; 35.61; 28.24, Boc; 24.78; 22.68; 22.23. Mass Spectrum (ISMS) m/z 419.2 (MH⁺), calculated for C₃₂H₄₅N₃O₅: 418 fragments (OR 65): 363.2, (-tBu).

The crude amine product 88 (1.7 g, ˜4 mmol) was dissolved in methanol/water and LiOH.H₂O (800 mg, 19 mmol) added. The solution was stirred at room temperature until the hydrolysis was complete (12 h) and then neutralised with 1M HCl (19 mL). On standing a copious white precipitate formed which was filtered off and washed with water. The solid was recrystallised from ethanol-water (˜95:5) to give fine needles of (mainly) the major diastereomer 89 (first crop 1 g), m.p.:175-177° C. The product was further recrystallised as required. ¹H NMR (300 MHz, CD₃OD): 6 (ref. 3.31 ppm) 7.33-7.18, 5H, m; 5.90, 1H, m; 5.35, 1H, d, J=17.1 Hz; 5.26, 1H, d, J=10.2 Hz; 4.31, 1H, m; 3.65, 1H, dd, J=5.7, 7.9 Hz; 3.27, 1H, m; 2.92, 1H, dd, J=5.2, 14.0 Hz; 2.76, 1H, dd, J=10.1, 14.0 Hz; 2.59, 1H, m; 1.82, 1H, m; 1.37, 9H, s, (Boc); 0.97, 3H, d, J=7 Hz; 0.94, 3H, d, J=7 Hz. ¹³C NMR (75 MHz, CD₃OD): 6 (ref. 49.15 ppm) 173.7; 159.4; 138.8; 134.5; 130.33; 129.8; 128.0; 120.5; 81.34; 63.65; 55.84; 41.19; 37.90; 32.70; 28.78; 26.11; 23.56. Mass Spectrum (ISMS) m/z 405 (MH⁺), calculated for C₂₃H₃₆N₂O₄: 404.

The amino acid 89 was esterified to 90 by the method of Bodansky and Bodansky(Bodansky and Bodansky 1984) as follows: the amino acid 89 (400 mg, 1 mmol) was dissolved in methanol/water and neutralised with Cs₂CO₃ (300 mg), then the solvents were removed in vacuo, then DMF added and removed in vacuo. The residue was dissolved in DMF (10 mL) and benzyl bromide (190 mg, 1.1 mmol, purified by passage through a short column of basic alumina) added to the stirred solution. After 2 h the reaction was diluted with aq. NaHCO₃ and extracted with 1:1 EtOAc:light pet. The organic layer was washed in turn with aq. NaHCO₃, water (×2), brine and then dried over MgSO₄. Evaporation of the solvent left the product 90 as a clear oil which solidified to a low melting solid (m.p. ˜55° C.) on standing (500 mg, ˜100%). TLC 25% EtOAc in light pet. Rf=0.57. ¹H NMR (300 MHz, CDCl₃): δ 7.38-7.32, 4H, m; 7.28-7.14, 6H, m; 5.82, 1H, m; 5.19-5.05, 4H, m's, (OBn ABq, J=12.5 Hz, δₐ=5.16, δ_{b=5.12} ppm); 4.9, 1H, br; 3.88, 1H, br; 3.44, 1H, bt, J=7 Hz; 2.88, 1H, dd, J=5, 14 Hz; 2.77-2.60, 2H, bm; 1.63, 1H, m; 1.56-1.35, m, 2H, 1.33, 9H, bs (Boc); 0.88, 3H, d, J=6.5 Hz; 0.85, 3H, d, J=6.5 Hz. ¹³C NMR (75 MHz, CDCl₃): δ 175.5; 155.5; 138.6; 135.8; 135.2; 129.2; 128.5; 128.2; 126.1; 117.4; 78.90; 66.40; 58.96; 58.49; 54.25; 42.83; 36.33; 35.71; 28.27 (Boc); 24.77; 22.63; 22.32. Mass Spectrum (ISMS) m/z 495 (M+H⁺), calculated for C₃₀H₄₂N₂O₄: 494

The amine 90 (500 mg, 1 mmol) was dissolved in ethyl acetate (20 mL) and 37% aqueous formaldehyde solution (0.5 mL) was added. The solution was stirred for 12 h and then diluted with light petroleum (40 mL) and washed in turn with aq. NaHCO₃, water (×2) and brine and then dried (MgSO₄). Removal of the solvent in vacuo gave the product 91 as a clear oil in approximately quantitative yield. Further purification was carried out by flash chromatography eluting with 10% ethyl acetate in light pet. ¹H NMR (500 MHz, CD₃CN): 0.6 (rotamers were present in a ratio of 7:3) 7.36, 4H, m, Ar; 7.27-7.11, 6H, Ar; 5.70, 1H, m, vinyl CH; 5.17-4.97, 4H, m's, vinyl CH₂ and OCH₂Ph; 4.44, 0.7H, d, J=5.0 Hz, ring CH₂(a), major rotamer; 4.33, 0.3H, d, J=4.4 Hz, ring CH₂(a), minor rotamer; 4.19, 0.7H, d, J=5.0 Hz, ring CH₂(b), major rotamer; 4.09, 0.3H, d, J=4.6 Hz, ring CH₂(b), minor rotamer; 4.06, 0.3H, m, Pheα, minor; 4.02, 0.7H, m, Pheα, major; 3.74, 0.7H, dd, J=9.8, 6.0 Hz, and 3.69, 0.3H, m, Leuα; 3.10, 1H, m, ring methine (homoallyl); 2.88, 0.3H, m, Pheβ(a); 2.84, 0.7H, dd, J=4.1, 13.4, Pheβ(a); 2.72, 0.3H, dd, J=6.5, 13.5, Pheβ(b); 2.65, 0.7H, dd, J=9.5, 13.2, Pheβ(b); 2.49, 1H, m, allyl(a); 2.15, 1H, m, allyl(b); 1.76-1.42, 3H, m's, Leuβ+γ; 1.33, 2.5H, s, Boc, minor rotamer; 1.09, 6.5H, s, Boc, major rotamer; 0.97-0.84, 6H, d's, Leuδ (major rotamer: 0.94, J=6.3 Hz; 0.90, J=6.2 Hz). ¹³C NMR (75 MHz, CD₃CN), only major rotamer reported except where indicated: δ (ref. 118.69 ppm) 173.3; 154.2; 140.9; 137.8; 136.3 (CH); 131.3; 129.9; 129.7; 129.6; 129.5; 127.2; 118.2 (CH₂); 79.98 (Boc tertiary); 67.17 (CH₂); 63.49 (CH); 62.47 (CH₂); 60.91 (CH); 57.68 (CH); 40.34 (CH₂); 36.04 (CH₂); 33.18 (CH₂); (29.08 Boc minor rotamer); 28.61 (Boc major rotamer); 25.98 (CH); 23.79 (CH₃); 22.36 (CH₃). Mass Spectrum (ISMS) m/z 507 (MH⁺), calculated for C₃₁H₄₂N₂O₄: 506

The alkene 91 was dihydroxylated with OsO₄/N-methylmorpholine-N-oxide in tBuOH/water as previously described for the dihydroxylation of 60. The crude product 92 was used directly in the next reaction. TLC 1:1 EtOAc:light pet. Rf=0.36. Mass Spectrum (ISMS) m/z 541 (M+H⁺), calculated for C₃₁H₄₄N₂O₆: 540

The glycol 92 (87 mg, 0.16 mmol) was dissolved in THF (4 mL) and H₅IO₆ (37 mg, 0.16 mmol) dissolved in THF (3 mL) was added and the reaction stirred at room temperature. A precipitate of iodic acid rapidly formed and the reaction was complete in <5 min. The THF solution was diluted with ether and washed in turn with 10% aq. Na₂CO₃, water, brine and then dried (MgSO₄). The product aldehyde 93 was of good purity but was not particularly stable to storage. Any traces of acid must be rigorously excluded to prevent isomerisation to the trans isomer. A portion was purified by flash chromatography, eluting with 15% EtOAc in light petroleum. TLC 15% EtOAc in light pet. Rf=0.27. The yield was good (>80%). Amide rotamers were evident in the NMR spectra, ratio ˜3:1, only the peak due to the main rotamer is reported unless otherwise noted. ¹H NMR (300 MHz, CD₃CN, ref 1.94 ppm): δ 9.53, 1H, s; 7.42-7.10, 10H, m's; 5.11, 2H, s, (OCH₂Ph); 4.41, 1H, br; 4.25, 1H, q, J=6.3 Hz; 4.15, 1H, br; 3.56, 1H, dt, J=8.5, 5.7 Hz; 3.54, 1H, bm; 2.90-2.58, 4H, m; 1.75-1.45, 3H, bm; 1.37, bs, Boc minor rotamer; 1.20, bs, Boc major rotamer; 0.92, 3H, d, J=6 Hz; 0.88, 3H, d, J=5.7 Hz. ¹³C NMR (75 MHz, CD₃CN, ref 118.69 ppm): δ 202.0; 173.1; 154.2; 140.4; 137.6; 131.1; 129.9; 129.62; 129.55; 127.26; 80.28 (Boc tertiary); 67.31 (CH₂); 61.90 (CH₂); 60.43 (CH); 58.56 (CH); 57.95 (CH); 43.75 (CH₂); 40.36 (CH₂); 36.48 (CH₂); 28.66 (Boc); 25.83 (CH); 23.67 (CH₃); 22.25 (CH₃). Mass Spectrum (ISMS) m/z 509 (MH⁺), calculated for C₃₀H₄₀N₂O₅: 508

Conversion of 4,5-cis aldehyde 93 to the 4,5-cis 4(S) amine product was completed by a two step reductive amination procedure as illustrated below:

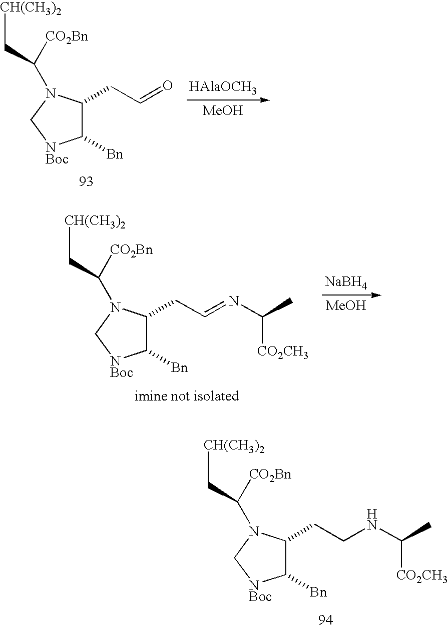

Alanine methyl ester hydrochloride (120 mg, 0.86 mmol) was dissolved in 1:1 brine:10% aq. Na₂CO₃ and extraction into CH₂Cl₂ (×2). The organic extracts were dried (MgSO₄), filtered and the majority of the solvent removed in vacuo to leave the volatile amine which was added to a solution of the freshly prepared aldehyde 93 (100 mg, 0.2 mmol) dissolved in methanol (˜7 mL, strictly acid free). The solution was stirred at room temperature for 2 h whereupon analysis of a test portion reduced with NaBH₄ showed imine formation to be complete (none of the alcohol formed on reduction of aldehyde was detected). Solid NaBH₄ (50 mg, 1.3 mmol) was added to the solution and stirring continued for 10 min and then the reaction partitioned between ethyl acetate and a water/brine/10% aq. Na₂CO₃ mixture. The aqueous phase was separated and the organic layer washed with water (×2) then brine and then dried (MgSO₄). NMR analysis of the crude product failed to detect the corresponding trans (S) diastereomer (<5%). Evaporation of the solvent left an oil which was purified by flash chromatography eluting with 20-40% EtOAc in light petroleum for a 60-70% yield of 94. TLC 40% EtOAc:light pet. Rf=0.43. Rotamers observed in the NMR spectra, ratio ˜3:1, separate signals due to the minor rotamer recorded only where indicated. ¹H NMR (300 MHz, CD₃CN, ref. 1.94 ppm): δ 7.37, 4H, m,; 7.3-7.1, 6H, m; 5.12, 5.09: 2H, ABq, J=12 Hz; 4.39 (major rotamer), 4.29 (minor): 1H, d, J=5 Hz; 4.15, 1H, J=5 Hz; 4.06, 1H, m, PheHα; 3.75-3.57, 4H, m, LeuHα+OCH₃; 3.25-3.10, 1H, m; 3.03, 1H, m; 2.87-2.60, 2H, m, Pheβ; 2.52-2.25, 2H, m; 1.81, 1H, m; 1.67, 1H, m; 1.6-1.38, 2H, m; 1.34, bs, Boc minor rotamer; 1.19, m, Alaβ; 1.15, bs, Boc major rotamer; 0.93, 3H, d, J=6.6 Hz; 0.89, 3H, d, J=6.3 Hz. ¹³C NMR (75 MHz, CD₃CN, ref. 118.69 ppm): δ 177.3; 173.4; 154.2; 141.0; 137.7; 131.2; 130.9; 129.9; 129.7; 129.6; 129.5; 127.1; 80.02 (Boc tertiary); 67.18 (CH₂); 62.55 (CH); 62.25 (CH₂); 60.75 (CH); 57.67 (2×CH, coincident signals); 52.55 (OCH₃); 45.96 (CH₂); 40.96 (CH₂); 36.15 (CH₂); 29.00 (Boc, minor rotamer); 28.73 (CH₂); 28.62 (Boc, major rotamer); 25.96 (CH); 23.66 (CH₃); 22.35 (CH₃); 19.7 (CH₃). Mass Spectrum (ISMS) m/z 596 (M+H⁺), calculated for C₃₄H₅₀N₃O₆: 595

Reductive amination of aldehyde 93 (or the 4,5-trans isomer) with NaBH(OAc)₃ in dichloroethane gave rise to a mixture of products 94 and 95 in the ratio 1:9.

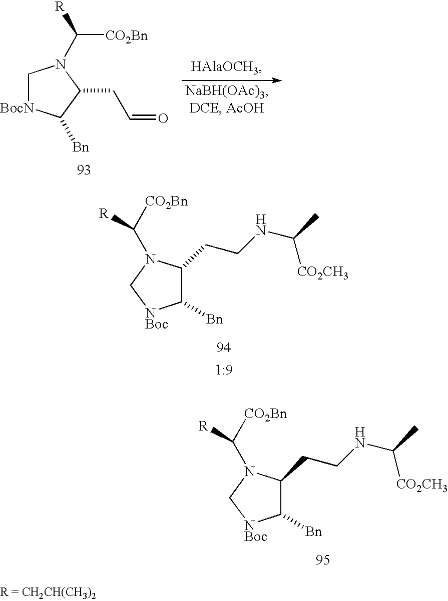

The aldehyde 93 (50 mg, 0.1 mmol) was dissolved in 1,2-dichloroethane (5 mL) and alanine methyl ester (˜2 equivalents) and acetic acid (1 drop, ˜14 mg) were added. The mixture was stirred at room temperature for 5 min and then NaBH(OAc)₃ (40 mg, 2 eq.) was added and stirring continued for 30 min. The solvent was then removed in vacuo and the residue partitioned between EtOAc and 10% aq. Na₂CO₃, the organic layer was washed with water and brine and then dried (MgSO₄). The product contained both diastereomers in the ratio ˜9:1, trans:cis. The products were purified by flash chromatography eluting with 20-45% EtOAc in light petroleum. TLC 40% EtOAc:light pet. Rf=0.43 (minor diastereomer, 94, cis), 0.23 (major diastereomer, 95, trans). Combined yield ˜60%. Rotamers were not observed although significant peak broadening was present, as observed for the corresponding trans aldehyde. The configuration of the major product was determined by NMR (NOESY experiment). ¹H NMR (300 MHz, CD₃CN, ref 1.94 ppm): δ 7.24-7.14, 10H, m's; 5.13, 2H, s, OCH₂Ph; 4.38, 1H, br, ring methylene(i); 3.97, 1H, bd, ring methylene(ii); 3.61, 3H, s, OCH₃; 3.75, 1H, ddd, J=2.7, 4.3, 8.7 Hz, PheHα; 3.50, 1H, m, LeuHα; 3.13, 1H, m, PheC′H(ring); 2.97-2.88, 2H, m, AlaHα+PheHβ(i); 2.72, 1H, dd, J=2.9, 8.7 Hz, PheHβ(ii); 2.33, 1H, ddd, J=11.5, 7.3, 5.5 Hz, CH₂NH(bridge)(i); 1.98, 1H, m (dt, overlaps with solvent peak), CH₂NH(bridge)(ii); 1.53, 2H, m, Leuβ+γ; 1.43, 9H(s)+1H(m), Boc+Leuβ; 1.35, 1H, m, bridge CH₂(i); 1.29, 1H, m, bridge CH₂(ii); 1.06, 3H, d, J=7.0 Hz, Alaβ; 0.88, 6H, m, Leuδ. ¹³C NMR (75 MHz, CD₃CN, ref 118.69 ppm): δ 177.2; 174.6; 154.5; 140.1; 137.6; 131.0; 129.9; 129.7; 129.6; 127.6; 80.61 (Boc tertiary); 67.50 (CH₂); 63.62 (CH₂); 63.5 (CH, br); 62.4 (CH, v.br); 60.67 (CH); 57.70 (CH); 52.47 (CH₂); 45.15 (CH₂); 40.65 (CH₂, v.br); 39.76 (CH₂); 32.81 (CH₂); 29.00 (CH₃, Boc); 26.21 (CH); 23.47 (CH₃); 22.88 (CH₃); 19.62 (CH₃). Mass Spectrum (ISMS) m/z 596 (MH⁺), calculated for C₃₄H₄₉N₃O₆: 595 The diastereomeric amines were converted to the protected γ-turn mimetic compounds 96 and 97 as described below:

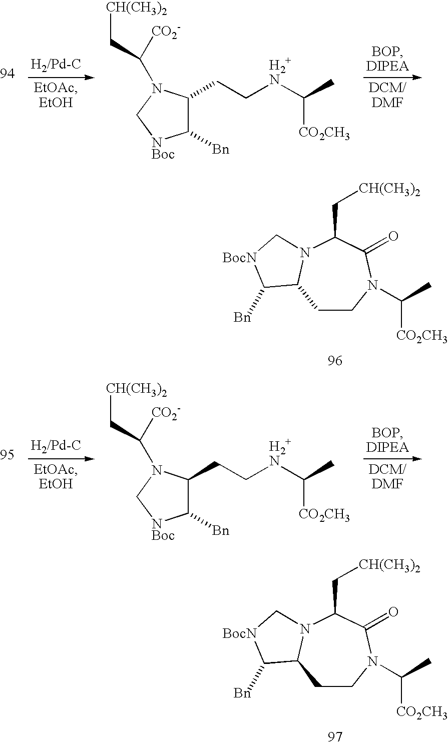

The 4,5-cis amine 94 (42 mg, 0.07 mmol) was dissolved in ethyl acetate:ethanol 10:3 (13 mL) and 35 mg of 10% palladium on activated carbon was added and the mixture hydrogenated at 32 psi H₂ for 3 h to deprotect the benzyl ester to the amino acid (MH⁺=506 Da). The solution was filtered and the solvent removed in vacuo, then the residue was dissolved in DMF (2 mL) and diluted with CH₂Cl₂ (15 mL) and DIEA (50 mg, ˜0.4 mmol) and BOP reagent (50 mg, 0.11 mmol) were added to the stirred solution at room temperature. The cyclisation was complete within a few minutes; the CH₂Cl₂ was then removed in vacuo and the residue diluted with ethyl acetate and washed in turn with 10% aq. Na₂CO₃/brine, water (×2), brine and then dried (MgSO₄) and the solvent removed in vacuo to leave a clear oil which was purified by flash chromatography eluting with 20% EtOAc in light petroleum for a yield of 25 mg (70%) of 96. TLC 1:1 EtOAc:light pet ˜0.45 The NMR spectra in CD₃CN at room temperature were significantly broadened indicating a degree of conformational interconversion slow on the NMR timescale. ¹H NMR (300 MHz, CD₃CN): δ 7.32-7.15, 5H, m, Ar; 4.88, 1H, q, J=7.1 Hz, Alaα; 4.20, 1H, bd, J=4.8 Hz, NCH₂N(a); 4.13, 1H, m, Pheα; 4.09, 1H, bd, J=5.0 Hz, NCH₂N(b); 3.72, 1H, m, Leuα; 3.65, 3H, s, OCH₃; 3.52, 1H, bdd, J=10.6, 15.2 Hz, bridge CH₂CH₂N(a); 3.30-3.21, 2H, m's, CH₂CH₂N(b) and PheC′H, 2.94, 1H, bm, Pheβ(a); 2.76, 1H, bm, Pheβ(b); 2.25 water peak; 1.9-1.4, 5H, e, Leuβ+γ and bridge CH₂CH₂N; 1.29, 3H, d, J=7.1 Hz, Alaβ; 3.25, 9H, vbr, Boc; 0.92, 6H, d, J=6.2 Hz, Leuδ. ¹³C NMR (75 MHz, CD₃CN): δ 173.5 (the amide and ester peaks appear to be co-incident); 154.9 (carbamate, br); 140.7; 130.7 (br); 129.6; 127.3; 80.54; 66.47; 63.83 (br); 62.36; 60.4 (very br); 56.29; 52.97; 44.77; (36.96, 36.40) very br, just resolved; 33.3 (very br); 28.78 (Boc, br); 26.86; 23.90 (br); 22.63; 15.47. Mass spectrum (ISMS) m/z 250.2 (M+H⁺), calculated for C₂₈H₃₇N₃O₆: 511 fragments (OR 60): 441, (-tBu); 397, (-Boc).

The synthesis of 97 was as for 96 but using the trans amine 95. TLC 1:1 EtOAc:light pet. Rf=0.53. The NMR spectra in CD₃CN were well resolved and rotamers were present in the ratio of 11:9; signals attributable to the same atom in the different rotamers are placed in parentheses where possible. ¹H NMR (300 MHz, CD₃CN, ref 1.94 ppm): δ 7.34-7.16, 5H, m; 4.69, 1H, m; 4.13, 1H, d, J=4.4 Hz; 3.92, 1H, m; (3.83, d, J=4.4; 3.79, d, J=4.4 Hz), 1H, 3.76-3.60, 2H, m's; (3.61, s; 3.81, s), 3H, OCH₃; 3.26, 1H, m; 3.15, 1H, m; 2.99, 1H, m; 2.77, 1H, m; 1.85-1.49, 3H, m's; (1.44, s; 1.41, s), 9H, Boc; 1.30, 3H, d, J=7.2 Hz, Alaβ; 1.36-1.24, 2H, m; 0.98-0.91, 6H, m. ¹³C NMR (75 MHz, CD₃CN, ref 118.69 ppm): δ 174.4; 173.3; 154.6; (140.54, 140.49); 130.7; 130.6; 129.8; 127.6; (80.65, 80.54), Boc tertiary; (66.12, 65.48, 65.21, 64.90) 2×CH; 60.67, CH₂; (56.82, 56.74), CH; (56.41, 56.24), CH; 52.87, CH₃; (46.19, 46.12), CH₂; (40.72, 39.84), CH₂; 39.16, CH₂; 30.44, CH₂; (29.03, 28.93) Boc; (25.64, 25.58), CH; 24.19, CH₃; 22.43, CH₃; 15.76, CH₃. Mass Spectrum (ISMS) m/z 488 (MH⁺), calculated for C₂₈H₃₇N₃O₆: 487

### Example (G)

### Acid Catalysed Isomerisation of Aldehydes 93

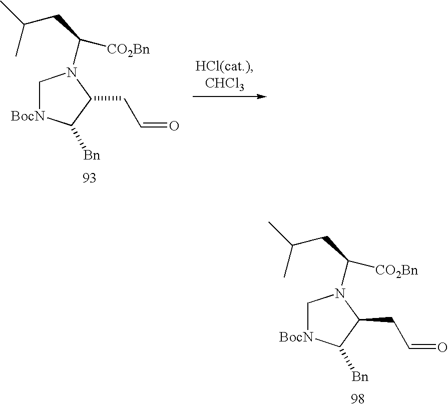

The trans (4(S)) aldehyde was obtained by the acid catalysed isomerisation of the cis diastereomer 93 in chloroform solution with catalytic HCl present. Significant decomposition to multiple unidentified by-products (most having high Rf) also occurs under the isomerisation conditions. The product was purified by flash chromatography eluting with 15% ethyl acetate in petroleum ether for a yield of about 35% 98 from crude 93. ¹H NMR (300 MHz, CD₃CN, ref. 1.94 ppm): δ 9.41, t, J=1.8 Hz; 7.45-7.10, 10H, m; 5.12, 2H, m, OCH₂Ph; 4.46, 1H, br; 4.01, 1H, bd; 3.82, 1H, m; 3.62-3.46, 2H, m; 2.95, 1H, bdd, J=13.0, 4.4 Hz; 2.81, 1H, dd, J=13.2, 8.0 Hz; 2.37, 2H, m (ABq of dd, J_{AB}=31, J_{ddA}=4.6, 1.8 Hz; J_{ddB}=7.2, 2.1 Hz), α-aldehyde; 1.75-1.25, 12H, e (1.4, bs, Boc); 0.9, 6H, bm. ¹³C NMR (75 MHz, CDCl₃): δ 202.9; 174.5; 154.4; 139.7; 137.5; 131.0; 129.9; 129.8; 129.7; 127.7; 80.81; 67.61; 64.03 (br); 63.18 60.49; 59.9 (br); 47.0 (br); 45.95; 39.88; 28.96 (Boc); 26.12; 23.25; 22.97.

### Example (H)

### Synthesis of a α-Turn Mimetic II(i)

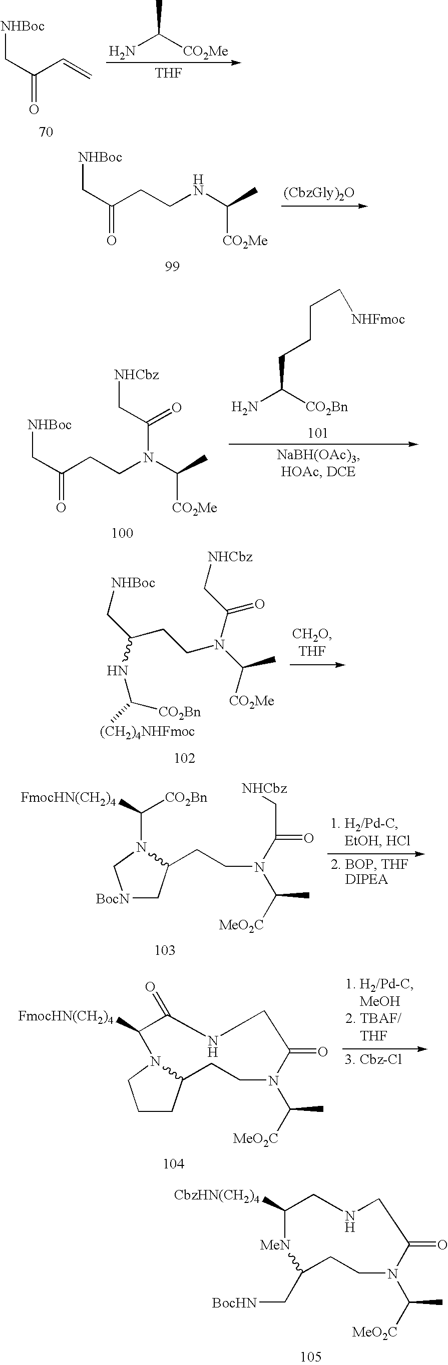

Compound 70 was prepared as described above, and reacted with alanine methyl ester to form 99 using the same method previously described for the synthesis of 71. The crude amino ketone 99 (1.22 g) was reacted with Cbz-glycine symmetric anhydride (synthesised from 1.95 g CbzGlyOH and 9.3 mLs 0.5M dicyclohexylcarbodiimide in dichloromethane) and 0.6 g DIEA in dichloromethane. The reaction was stirred at room temperature for 10 hours then diluted with ether (any DCU precipitate was filtered off) and the ether solution was washed with 1M HCl, aqueous sodium bicarbonate then brine and then dried over magnesium sulfate (removed by filtration) and the volatiles removed under reduced pressure to leave the crude product as an oil which was purified by flash chromatography eluting with 2:1 ethyl acetate:light petroleum ether, yield of 100 was 1.8 g (90%). Reductive amination of 100 with 101 derived from the deprotection of BocLys(Fmoc)OBn (TFA, CH₂Cl₂) is carried out by the previously described method for the formation of 73 (71% yield after flash chromatography eluting with 2:1 to 3:1 ethyl acetate:light petroleum). The product amine 102 was dissolved in ethyl acetate and formalin added to the stirred solution resulting in the formation of imidazolidine 103. The ethyl acetate solution was washed with aqueous sodium bicarbonate, water (twice), brine and then dried over magnesium sulfate (removed by filtration) and the volatiles removed under reduced pressure to leave the crude product as an oil which was purified by flash chromatography eluting with 3:2 ethyl acetate:light petroleum ether (yield>75%). The protected pre-cyclisation compound 103 (400 mgs) was dissolved in 0.1M ethanolic HCl (20 mLs) and hydrogenated with 250 mgs of 10% Pd—C. The hydrogenation was complete after 7 hours (about 40 psi H₂, room temperature). The solution was filtered through a celite pad to remove the catalyst and 50 mLs of DMF added. Volatiles (ethanol) were removed under reduced pressure then a solution of BOP reagent (300 mgs) and DIEA (300 mgs) in 150 mLs of DMF was added and the mixture stirred at room temperature for 15 minutes. Most of the DMF was removed under reduced pressure and the residue dissolved in ethyl acetate and washed with 1M HCl, aqueous sodium bicarbonate, water (twice), brine and then dried over magnesium sulfate (removed by filtration) and the volatiles removed under reduced pressure to leave about 300 mgs of crude product 104. The crude product was dissolved in 30 mLs methanolic HCl (0.1M) and hydrogenated (200 mgs Pd—C, 40 psi H₂) for 24 hours reducing the imidazolidine to an N-methyl group. The catalyst was filtered off (celite) and the solvent removed under reduced pressure, the residue was then treated with tetrabutylammonium fluoride in THF to remove the FMOC group. The free amine was then reprotected by addition of benzyl chloroformate (65 mgs) and DIEA (100 mgs). After stirring for 1 hour ethyl acetate was added and the organic layer was washed with 1M HCl, water, then brine, dried over magnesium sulfate (removed by filtration) and the volatiles removed under reduced pressure to leave an oil which was purified by flash chromatography eluting with 3-5% ethanol in chloroform for a yield of about 40% of 105 based on 103.

### Example 1

### Reagents, Solvents and General Laboratory Methodology Used in Examples J to BK

Commercial-grade reagents and solvents were used without further purification except as indicated. Oven-dried glassware was used in all reactions carried out under an inert atmosphere (refers to either dry argon or dry nitrogen). “Removal of the solvent (or volatiles) by evaporation under reduced pressure” was carried out by rotary evaporation on a low vacuum pump. Analytical TLC was performed on alumina-backed Merck Keisegel 60 F254 silica gel plates, and visualised using ultraviolet light or potassium permangante dip. Column chromatography was performed using 230-400 mesh Merck Silica Gel 60 under an atmospheric pressure unless stated. Flash column chromatography refers to the use of a positive pressure of nitrogen.

### Example J

### Reverse-Phase High-Performance Liquid Chromatography (HPLC)

Pentapeptide building block intermediates were analysed by analytical RP-HPLC performed on a Agilent 1100 series assembly equipped with an auto-injector and a detector with deuterium light source using a Phenomenex Synergi MAX-RP 80 Å column (50 mm×2.00 mm×4 μm). Data was recorded and processed with Agilent ChemStation for LC software. A solvent gradient of 10%/min of solvent B in solvent A (solvent A=H₂O/0.05% TFA, solvent B=90% CH₃CN/10% H₂O/0.05% TFA) was used at a flow rate of 1 mL/min over 9 min. The eluent was monitored by a UV detector at 220, 236, 252, 268, and 284 nm.

Peptide samples were analysed by analytical RP-HPLC performed on a Shimadzu LC2010A system equipped with an auto-injector and a detector with deuterium light source using a Phenomenex Jupiter C18 300A column (250×4.60 mm, 10 micron). Data was recorded and processed with Shimadzu Class VP software. Unless stated otherwise a 1% per min linear gradient of solvent A with 0-40% solvent B was employed at a flow rate of 1 mL/min over 40 min. The eluent was monitored by a UV detector at 214 nm and 280 nm.

Purification of the peptides was achieved using semi-preparative RP-HPLC on a Shimadzu LC20AT solvent delivery system with a Phenomenex Jupiter C18 300A column (250×10.00 mm, 10 micron) at a flow rate of 7 mL/min and a 1%/min linear gradient of 0-50% B over 50 min. The absorbance was monitored with a Shimadzu SPD-10AVP detector with a deuterium light source at 214 nm and 208 nm. Data was recorded using the Shimadzu Class VP software, and fractions collected during RP-HPLC purification were analyzed by mass spectrometry. Fractions containing the desired mass were further analyzed by analytical RP-HPLC and pure material was pooled and lyophilized.

### Example K

### CD Spectroscopy

CD experiments were performed on a Jasco Model J-810 spectropolarimeter, which was routinely calibrated with (1S)-(+)-10-camphorsulfonic acid at a concentration of 0.06% (w/v). Experiments were performed at 25° C. using a 0.1 cm cell, a scan speed of 10 nm/min from 260 to 180 nm, with a resolution of 1 nm and a response time of 0.25 s. The spectra were averaged over 5 scans, baseline subtracted from the corresponding blank conditions, and smoothed using the Savitsky-Golay smoothing algorithm (Savitsky, A. and Golay, M. J. E., 1964, Anal. Chem, 36, 1627-1639) to reduce noise. The samples were dissolved in 10 mM phosphate buffer, measured as pH 7.4, and when required the appropriate volume of TFE was added to adjust to 30% TFE. The concentration of the samples of peptides incorporating Helix Mimetic A (galanin 1-16 analogs) were determined using the Beer-Lambert Law by measuring the absorbance of the samples at 280 nm and using a molar extinction co-efficient of ε₂₈₀=6620 M⁻¹cm⁻¹ (ε₂₈₀ of Galanin 1-30, contains same number of Trp and Tyr as Galanin 1-16). The concentration of the samples of peptides incorporating Helix Mimetic B (Temporin A analogs) samples and the pentapeptide 13-membered ring Helix Mimetic A and B samples were determined by quantitative RP-HPLC against a standard of known concentration. All samples were between 40-200 μM, and CD spectra were recorded at a range of concentrations to check that sample aggregation was not occurring. Raw CD data was recorded in raw ellipticity units and converted to mean ellipticity per mole using the equation [θ]=θ/(10×c×l), where c is the sample concentration (M) and l is the cell path length (cm). Data was not converted to mean peptide molar ellipticity per residue as for compounds containing the covalent ethyl-bridge hydrogen bond replacement it is unclear how many “natural” peptide units to count in these calculations. Variable temperature CD experiments were carried using a CDF-426/L Peltier type CD/Fluorescence simultaneous measurement attachment, and samples were equilibrated for 10 min once the desired temperature was reached.

### Example L

### NMR Spectroscopy

Spectra were recorded on Bruker Advance DRX-600 MHz spectrometer at 298 K unless otherwise stated and processed using Topspin (Bruker Corp. Billerica, Mass., USA) software or recorded on a Varian 400 MHz 54 ASC spectrometer and processed using iNMR software (Nucleomatica, Molfetta, Italy). The chemical shifts are reported in parts per million (ppm) on the δ scale. Solvents used for NMR analysis (reference peaks listed) included: DMSO-d₆ (CHD₂SOCD₃ at δ_{H} 2.60 ppm, CDCl₃ (CHCl₃ at δ_{H} 7.26 ppm, and 2,2-dimethyl-2-silapentane-5-sulfonic acid (0.0 ppm) as an internal standard for D₂O/H₂O samples. The multiplicity of each signal for ¹H NMR is indicated by s (singlet), br s (broad singlet), d (doublet), t (triplet) and m (multiplet). Coupling constants (J) are quoted in Hz and recorded to the nearest 0.1 Hz. The peptide concentration for the ¹H NMR measurements was between 1-1.5 mM in 90% H₂O/10% D₂O (v/v). 2D NMR spectra were recorded in phase-sensitive mode using time proportional phase incrementation. TOCSY experiments were carried out using a MLEV-17 spin lock sequence with a mixing time of 80 ms, and NOESY with a mixing time of 300 ms. Water suppression was achieved using watergate W5 pulse sequences with gradients using double echo, and spectra were acquired over 7184 Hz.

### Example M

### Mass Spectrometry

Electro-Spray Ionisation Mass Spectrometry (ESI-MS): Micro mass spectra were obtained using a LCT-TOF mass spectrometer (MicroMass, Manchester, UK) equipped with an electro-spray ionisation source. Samples (5-15 μL) were injected into solvent flowing at 100 μL/min (75% acetonitrile (aq)/0.1% formic acid (aq)). The orifice potential was set to 90 V, the focusing potential to 280 V, declustering potential to 20 V, and spectra were acquired over the range 200 to 1800 m/z.

ESI Nanospray MS/MS: Samples were analysed by nanospray infusion electrospray ionization (ESI)-tandem mass spectrometry (MS/MS) in positive mode by using an ESI mass spectrometry quadruple-time-of-flight Qstar Pulsar system (Applied Biosystems, Foster City, Calif., USA). The [M+2H]²⁺ ion was selected for MS/MS analysis.

MALDI MS/MS: Samples were analysed on an Model 4700 Proteomics Bioanalyser (Applied Biosystems, Foster City, Calif., USA). Samples were spotted onto a MALDI plate with an equal volume of matrix (15 mg/mL 1,5 di-amino napthelene 0.1% TFA/50% ACN) and allowed to air dry. MALDI-TOF spectra were acquired automatically in reflector positive operating mode with source voltage 20 kV and Grid1 voltage 12 kV, collecting 15000 shots with a random laser pattern. MS/MS analysis was performed on the major ion, by acquisition in automatic mode with source voltage 8 kV and Grid1 voltage 6.8 kV, and 12,500 shots. Data was interpreted using Data Explorer software.

### Example N

### Solid-Phase Peptide Synthesis (SPPS)

All peptides were assembled by manual SPPS using standard protocol for Fmoc chemistry (Novabiochem) unless otherwise stated. The individual coupling steps were performed in screw-cap glass reaction vessels, fitted with a sintered glass frit, which were agitated by a mechanical shaker. The Rink amide MBHA resin was washed and allowed to swell in DMF for at least 2 hours prior to commencing synthesis. Relative to the molar loading of the resin, four equivalents of N_{α}-Fmoc protected amino acids activated with 4 equivalents of 0.5M HBTU in DMF and 4 equivalents of DIEA were used for the individual 1 min coupling steps. The Fmoc group was removed using 20% piperidine/DMF (v/v) (2×5 min). After each 10 min peptide coupling reaction a resin sample (˜5 mg) was withdrawn and the coupling efficiency determined by the quantitative ninhydrin reaction. If coupling was lower than 99.6% the amino acid was re-coupled. If this second coupling was again lower than 99.6% the resin was acetylated with a solution of acetic anhydride (870 μL) and DIEA (470 μL) in DMF (15 mL).

Synthesis of the H-bond mimeic peptides was achieved by coupling of the pentapeptide helix mimetic building block at the appropriate stage in SPPS chain assembly using a modified coupling procedure. Relative to the molar loading of the resin, 0.5-0.7 equivalents of the pentapeptide unit were activated with equimolar amounts of 0.5M HATU in DMF and DIEA, and reacted overnight. The resin was drained, and the filtrate analysed using mass spectrometry to check for any un-coupled pentapeptide starting material. The resin was washed with DMF, the N_{α}-terminal Fmoc group removed, and SPPS chain assembly continued using the standard protocol. As the resin was not acetylated following coupling of <1 equivalents of pentapeptide building-block, there were two major products following final resin cleavage—the desired peptide containing the mimetic and the shorter peptide product missing the pentapeptide unit.

Peptides were stored on-resin containing a N_{α}-terminal Fmoc group. Immediately prior to cleavage the N_{α}-terminal Fmoc group was removed using 20% piperidine/DMF (v/v), the resin washed with DMF and then DCM, and dried under flow vacuum with a nitrogen atmosphere. Cleavages were performed by treating the dried peptide resin with TFA/H₂O/triisopropylsilane (95:5:5 v/v) for 2 h at 0° C. Volatiles were evaporated under a flow of nitrogen, and the residue treated with cold diethyl ether to precipitate the peptide. The precipitate was filtered, dissolved in buffer A:B (1:1 v:v) and lyophilized to give the crude product.

### Example O

### Synthesis of Pentapeptide Helix Mimetic A: Weinreb Amide Formation to Give Benzyl 2-(methoxy(methyl)amino)-2-oxoethylcarbamate (201)

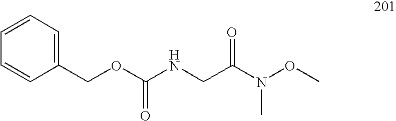

A solution of Cbz-Gly-OH (9.296 g, 44.5 mmol), N,O-dimethylhydroxylamine (6.505 g, 66.7 mmol), HBTU (20.241 g, 53.4 mmol), DIEA (15.507 mL, 89 mmol) and DMF (100 mL) was stirred at rt overnight. The solvent was removed under reduced pressure, and the residue dissolved in 1/1 v:v ethyl acetate:1M aqueous HCl. The organic layer was washed with 1M aqueous HCl, saturated aqueous sodium bicarbonate, then saturated brine (three times), dried over magnesium sulfate, filtered, and evaporated under reduced pressure to give the Weinreb amide (6.632 g, 59%) as a white solid.

### Example P

### Synthesis of Pentapeptide Helix Mimetic A: Vinyl Grignard Reaction to Form Benzyl 2-oxobut-3-enylcarbamate (202)

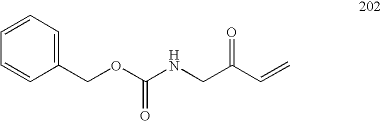

Magnesium turnings (6.0 g) were ground using a mortar and pestle and added to a 3-neck round bottom flask fitted with a pressurised dropping funnel and ice trap under an inert atmosphere. Enough THF was added to just cover the magnesium turnings along with a small amount (˜5 mg) of iodine. Vinyl bromide (17 mL) in THF (30 mL) was added into the dropping funnel, and this was added slowly over a period of 0.5-1 hour. Gradually additional THF was added to adjust the total volume of THF used to 250 mL. Once all the magnesium turnings had been consumed (˜2 h) the reaction was assumed as complete and the yield quantitative, to give a 1M vinyl magnesium bromide solution.

To a solution of Cbz-Gly-Weinreb amide 201 (6.590 g, 26.2 mmol) in THF at 0° C. was added vinyl magnesium bromide (66 mL of a 1M solution, 66 mmol, prepared as above) in two portions over one hour. The solution was allowed to warm to room temperature, stirred for 2 h, then quenched by addition of 1M aqueous HCl/ice. The aqueous layer was extracted three times with dichloromethane, dried over magnesium sulfate, filtered, and the solvent removed under reduced pressure to give 202 (5.543 g, 97%) as a yellow oil. To prevent polymerisation, 202 was stored at 0° C. as a 0.5 M solution in dichloromethane. ¹H NMR (600 MHz, CDCl₃) δ 7.25-7.29 (m, 5H, ArH), 6.31 (m, 2H, CH═CH₂), 5.85 (dd, J=4.1, 10.2 Hz, 1H, CH═CH₂), 5.76 (br s, 1H, NH), 5.08 (s, 2H, OCH₂), 4.23 (d, J=4.3 Hz, 2H, NHCH₂)¹³C NMR (160 MHz, CDCl₃) δ 194.27, 156.00, 136.14, 133.21, 129.50, 128.24, 127.86, 127.77, 66.65, 48.39. [M+H]⁺=371.2Analytical HPLC: t_{R}=5.14 min

### Example Q

### Synthesis of Pentapeptide Helix Mimetic A: Conjugate Addition of Gly-OtBu to Form tert-Butyl 2-(4-(benzyloxycarbonylamino)-3-oxobutylamino)ethanoate (203)

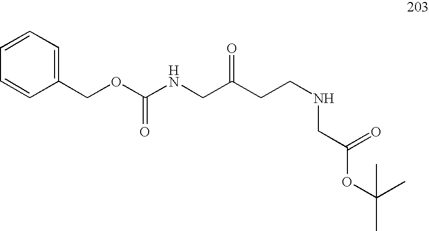

To a solution of glycine tert-butyl ester hydrochloride salt (4.234 g, 25.3 mmol), DIEA (9.1 mL, 55.7 mmol), and dichloromethane (30 mL) was added a solution of benzyl 2-oxobut-3-enylcarbamate 202 in dichloromethane (50.6 mL of a 0.5 M solution, 25.3 mmol). The reaction was checked by mass spectrometry after stirring at rt for 20 min, and showed the product peak of 203 at [M+H]⁺=351 and a small peak at [M+H]⁺=570, indicating some tertiary amide di-adduct side-product had formed. This solution was used without purification. [M+H]⁺=350.9

### Example R

### Synthesis of Pentapeptide Helix Mimetic A: Acylation with Boc-Leu-OH to Give (S)-tert-Butyl 2-(N-(4-(benzyloxycarbonylamino)-3-oxobutyl)-2-(tert-butoxycarbonylamino)-4-methylpentanamido)acetate (204)

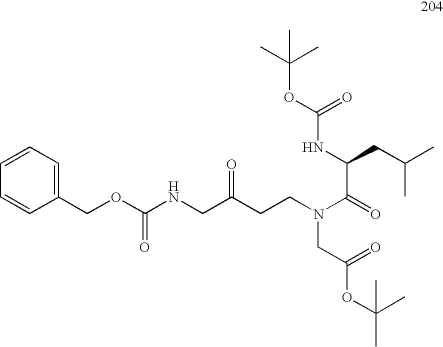

To a crude solution of 203 (assumed 100% yield, 25.3 mmol) was added Boc-Leu-OH.H₂O (15.737 g, 63.2 mmol), HATU (19.213 g, 50.6 mmol), and DIEA (13.2 mL, 75.9 mmol), and the mixture stirred at rt overnight. The solvent was removed under reduced pressure and the residue dissolved in ethyl acetate/water (1:1 v:v). The aqueous layer was extracted twice with ethyl acetate, the organic layers combined, and then washed with saturated aqueous sodium bicarbonate, saturated ammonium chloride, and saturated brine, dried over magnesium sulfate, filtered, and the solvent evaporated under reduced pressure. The residue was purified by silica gel flash column chromatography (2:1, dichloromethane:ethyl acetate) to give 204 (6.837 g, 48%) as a yellow oil. ¹H NMR (600 MHz, CDCl₃, rotamers listed, integrals correspond to two rotamers) δ 7.34-7.33 (m, 10H, ArH), 5.60 (br s, 1H, NHgly), 5.46° (br s, 1H, NHgly), 5.09 (m, 6H, OCH₂, NHLeu (two rotamers)), 4.56 (m, 1H, CHLeu), 4.43 (d, J=18.7 Hz, 1H, NCHHCO), 4.32 (m, 1H, CHLeu), 4.14 (d, J=17.1 Hz, 1H, NCHHCO), 4.08 (m, 2H, NHCHH), 4.03 (m, 2H, NHCHH), 3.93 (d, J=18.7 Hz, 1H, NCHHCO), 3.80-3.54 (m, 4H, COCH₂CH₂), 3.73 (d, J=17.1 Hz, 1H, NCHHCO), 2.97-2.72 (m, 4H, COCH₂CH₂), 1.72 (m, 1H, CHHLeu), 1.65 (m, 1H, CHHLeu), 1.53-1.32 (m, 44H, CH₂Leu, CHLeu, C(CH₃)₃), 0.97-0.87 (m, 15H, CH₃Leu). High temperature NMR (DMSO, 333K) minimised but did not completely abolish rotamers. ¹³C NMR (150 MHz, CDCl₃, all rotamers) δ 204.52, 203.91, 174.04, 173.53, 168.60, 168.21, 156.16, 156.04, 155.66, 155.44, 136.24, 128.44, 128.08, 127.98, 82.60, 81.77, 79.69, 79.52, 77.21, 77.00, 76.79, 66.93, 66.90, 51.96, 50.79, 50.55, 49.95, 48.57, 48.39, 43.58, 43.24, 42.28, 42.03, 39.41, 38.43, 28.27, 28.24, 27.95, 27.94, 24.53, 24.38, 23.38, 23.33, 21.65, 21.55. High temperature NMR (DMSO, 333K) minimised but did not completely abolish rotamers. [M+H]⁺=564.4Analytical HPLC: t_{R}=8.63 min

### Example S

### Synthesis of Pentapeptide Helix Mimetic A: Replacement of Cbz with Fmoc Protecting Group, Boc Deprotection, Acylation with Alloc-Leu-OH to give (205)

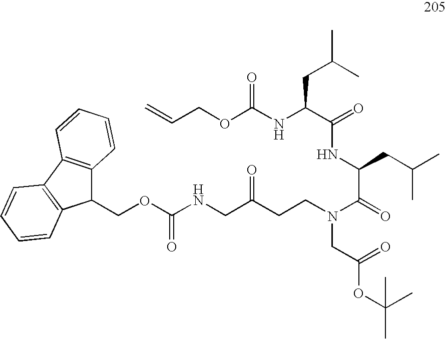

A solution of 204 (6.868 g, 12.2 mmol), 0.1M aqueous HCl (98 mL), 10% Pd/C (300 mg), and ethanol (600 mL) was reacted in a Parr Hydrogenator at 40 p.s.i. for 1 h. The solution was filtered through celite and the solvent removed under reduced pressure to give a pale yellow solid (5.381 g, 95%) as the hydrochloride salt. To this solid (4.525 g, 9.7 mmol) dissolved in 1:1 v:v dioxane:water (each 150 mL) was added Fmoc-succinimide (3.613 g, 10.7 mmol) and DIEA (4.3 mL, 42.2 mmol), and the resulting solution stirred overnight at rt. Water was added and the aqueous layer extracted twice with ethyl acetate. The combined organic layers were washed with saturated aqueous sodium bicarbonate, dried with magnesium sulfate, filtered, and the solvent removed under reduced pressure. The residue was purified by silica gel flash column chromatography (3:1, dichloromethane:ethyl acetate) to give a white solid (4.624 g, 73%). To this white solid (2.995 g, 4.6 mmol) at 0° C. was added 4M hydrochloric acid in dioxane (80 mL), and the solution stirred at 0° C. for 2 h. The solvent was removed under reduced pressure to give the Boc-deprotected hydrochloride salt (2.76 g, 99%) as a pale yellow solid. To this material (2.76 g, 4.7 mmol) was added Alloc-Leu-OH (1.149 g, 5.3 mmol), EDC (1.165 g, 6.1 mmol), HOBt (954 mg, 7.1 mmol), DIEA (1.6 mL, 15.7 mmol), and dichloromethane (200 mL) and the resulting solution stirred overnight at rt. Dichloromethane was added, the organic layer was washed twice with saturated brine, the aqueous layer back extracted with dichloromethane, and the combined organic layers dried over magnesium sulfate, filtered, then evaporated under reduced pressure. The residue was purified by silica gel flash column chromatography (1:1, petroleum ether:ethyl acetate) to give 205 (3.101 g, 87%) as a white solid. ¹H NMR (600 MHz, (CD₃)₂SO, 60° C., mixture of rotamers) δ 7.85 (d, J=7.5 Hz, 2H, ArH), 7.83 (m, 1H, NH Leu rotamer), 7.79 (m, 1H, NH Leu), 7.69 (m, 2H, ArH), 7.41 (t, J=7.4 Hz, 2H, ArH), 7.34 (m, 1H, NH gly), 7.33 (t, J=7.4 Hz, 2H, ArH), 7.09 (m, 1H, NH Leu), 5.90 (m, 1H, ═CH), 5.28 (m, 1H, ═CHH), 5.16 (m, 1H, ═CHH), 4.80 (m, 1H, NHCH Leu), 4.50-4.45 (3H, OCH₂ alloc, NCHHCO), 4.32 (d, J=6.5 Hz, 2H, CH₂ Fmoc), 4.23 (m, 1H, CH Fmoc), 4.08-3.96 (m, 3H, NCHHCO, NCHHCO rotamer, NHCH Leu), 3.88-3.82 (m, 2H, NHCH₂), 3.76 (m, 1H, NCHHCO rotamer), 3.60 (m, 2H, COCH₂CH₂), 3.46 (m, 2H, COCH₂CH₂ rotamer), 2.85 (m, 2H, COCH₂CH₂), 2.63 (m, 2H, COCH₂CH₂ rotamer), 1.65-1.39 (m, 15H, CH Leu, CH₂ Leu, C(CH₃)₃), 0.91 (12H, CH₃ Leu)¹³C NMR (150.9 MHz, (CD₃)₂SO, 60° C., mixture of rotamers) δ 205.91, 205.30, 172.21, 172.19, 172.13, 168.96, 168.16, 156.40, 156.37, 155.60, 143.81, 140.74, 133.59, 127.61, 127.05, 125.19, 120.09, 116.85, 81.55, 80.63, 65.73, 54.87, 52.89, 50.36, 49.98, 49.89, 48.80, 46.64, 42.95, 42.61, 40.84, 40.75, 38.35, 37.30, 30.64, 27.64, 24.16, 24.04, 23.22, 23.20, 23.08, 21.31 [M+H]⁺=749.4 Analytical HPLC: t_{R}=9.38 min

### Example T

### Synthesis of Pentapeptide Helix Mimetic A: Preparation of N-Boc-Tyr(2-Br-Z)-O-allyl ester (206)

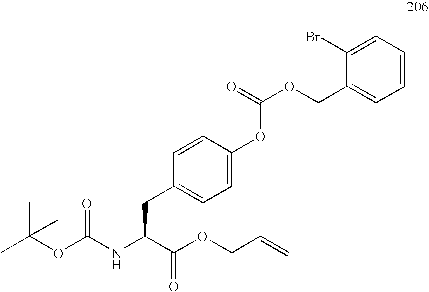

To solution of N-Boc-Tyr(2-Br-Z)-OH (5.0 g, 10.12 mmol) in dichloromethane (50 mL) at 0° C. was added allyl alcohol (512 mg, 8.8 mmol), DMAP (11 mg, 0.09 mmol) and EDC (1.855 g, 9.67 mmol). The reaction was stirred for 2 h at 0° C., then stirred overnight at rt. The solvent was evaporated, dissolved in ethyl acetate/H₂O (1:1 v:v), and the organic layer separated and washed with saturated aqueous sodium bicarbonate, water, dried with magnesium sulfate, filtered, and the solvent removed under reduced pressure. The residue was purified by silica gel flash column chromatography (1:1, petroleum ether:ethyl acetate) to give N-Boc-Tyr(2-Br-Z)-O-allyl ester 206 (4.511 g, 8.45 mmol, 96%) as a white solid. ¹H NMR (400 MHz, CDCl₃) δ 7.61 (d, J=8.0 Hz, 1H, ArH), 7.50 (d, J=7.6 Hz, 1H, ArH), 7.35 (t, J=7.5 Hz, 1H, ArH), 7.23 (t, J=7.7 Hz, 1H, ArH), 7.14 (m, 4H, ArH), 5.85 (m, 1H, CH═CH₂), 5.37 (s, 2H, OCH₂Ar), 5.27 (m, 2H, CH═CH₂), 4.99 (m, 1H, NHCH), 4.60 (d, J=5.5 Hz, 2H, OCH₂CH), 3.10 (m, 2H, CHCH₂Ar), 1.42 (s, 9H, C(CH₃)₃) ¹³C NMR (150.9 MHz, CDCl₃) δ 206.93, 153.34, 150.14, 134.32, 134.21, 133.95, 132.95, 131.37, 130.41, 130.19, 130.11, 127.63, 123.50, 121.00, 119.10, 69.62, 66.01, 54.37, 37.69, 30.92, 28.28; [M+H]⁺=534/536

### Example U

### Synthesis of Pentapeptide Helix Mimetic A: Preparation of Tyr(2-Br-Z)-O-allyl ester (207)

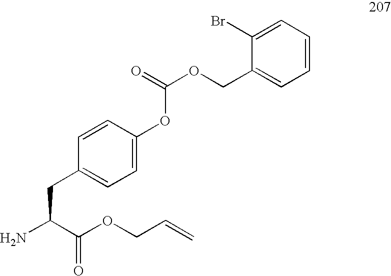

To a solution of N-Boc-Tyr(2-Br-Z)-O-allyl ester 206 (1.335 g, 2.5 mmol) in dichloromethane (5 mL) was added 5 mL of TFA. The solution was stirred for 30 min at rt, the volatiles removed under reduced pressure, and the residue dissolved in dichloromethane. To neutralise the TFA salt the organic layer was washed twice with saturated aqueous sodium bicarbonate, dried using magnesium sulfate, filtered, and the solvent removed under reduced pressure to give Tyr(2-Br-Z)-O-allyl ester 207 (1.071 g, 99%) as a yellow oil. [M+H]⁺=433.9/435; Analytical HPLC: t_{R}=6.26 min

### Example V

### Synthesis of Pentapeptide Helix Mimetic A: Reductive Amination with Tyr(2-Br-Z)-O-allyl ester to Form (2S,9S,12S)-allyl 4-((((9H-fluoren-9-yl)methoxy)carbonylamino)methyl)-2-(4-((2-bromobenzyloxy)carbonyloxy)-benzyl)-7-(2-tert-butoxy-2-oxoethyl)-9,12-di isobutyl-8,11,14-trioxo-1 5-oxa-3,7,10,13-tetraazaoctadec-17-en-1-oate (208a, 208b)

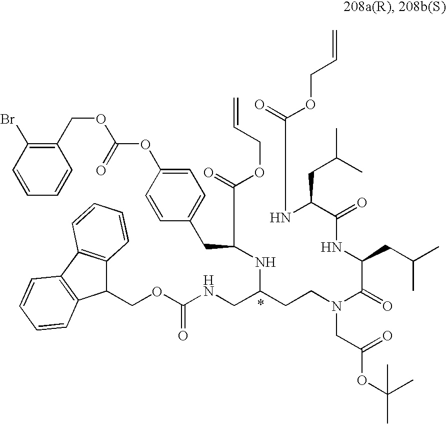

A solution of ketone 205 (1.3 g, 1.7 mmol), Tyr(2-Br-Z)-O-allyl ester 207 (852 mg, 2 mmol), sodium triacetoxyborohydride (921 mg, 4.3 mmol), and dichloroethane (8 mL) was stirred overnight at rt. Ethyl acetate was added and the organic layer washed four times with saturated aqueous sodium bicarbonate, dried with magnesium sulfate, filtered, and the solvent removed under reduced pressure. The residue was purified by silica gel flash column chromatography (2:1, dichloromethane:ethyl acetate) to give a mixture of 208a/208b in a 1.7:1 ratio (1.17 g, 59%) as a white solid. The 208a(R)/208b(S) mixture (1.7:1) was inseparable, and appeared as one spot on TLC and one peak on HPLC. NMR spectroscopy was recorded at 60° C. in (CD₃)₂SO; this reduced but did not completely eliminate rotamers. The RIS ratio was assigned where 4 peaks (2×rotamers 2×diastereoisomers) coalesced into 2 peaks (2×diastereoisomers) at 60° C. (2-Br-Z OCH₂ singlet resonance, and to some extend ^{t}Bu singlet resonance). The diastereoisomer ratio was assigned absolutely as R and S using 2D NMR spectra of the purified, cyclised material (see 10a and 10b), with the assumption that the relative isomer ratio was consistent. ¹H NMR (600 MHz, (CD₃)₂SO, 60° C., mixture of diastereoisomers and rotamers, no integrals listed) δ 7.86 (m, ArCH), 7.82 (m, NH Leu), 7.75 (m, NH Leu), 7.67, (m, ArCH), 7.55 (m, ArCH), 7.45 (m, ArCH), 7.41 (m, ArCH), 7.32 (m, ArCH), 7.27 (m, ArCH), 7.11 (m, ArCH), 7.07 (m, NH Leu), 7.02 (m, NH gly), 5.88 (═CH), 5.76 (═CH), 5.33 (s, CH₂ 2BrZ), 5.32 (s, CH₂ 2BrZ), 5.28-5.10 (m, ═CH₂), 4.87 (CH Leu), 4.49-4.43 (m, CH₂ alloc, CH Leu, NCHHCO), 4.30 (m, CH₂ Fmoc), 4.23 (m, CH Fmoc), 4.09 (CH Leu), 3.97-3.85 (m, NCHH, NCHHCO), 3.69-3.64 (m, NCHH, CH tyr), 3.39 (m, CH₂ tyr), 2.99-2.81 (m, NHCH₂, CH₂ tyr), 2.62 (m, C*H), 2.56 (m, C*H), 1.69-1.47 (m, CH Leu, CH₂ Leu, C*CH₂), 1.42 (s, C(CH₃)₃), 1.37 (s, C(CH₃)₃), 0.91 (m, CH₃ Leu), 0.84 (m, CH₃ Leu).

¹³C NMR (150.9 MHz, (CD₃)₂SO, 60° C., mixture of diastereoisomers and rotamers) δ 182.91, 182.76, 182.59, 181.29, 181.23, 180.01, 179.57, 177.99, 177.32, 177.30, 177.28, 165.63, 164.80, 161.99, 161.96, 161.89, 161.88, 158.65, 158.63, 158.34, 153.18, 153.14, 151.94, 149.99, 148.72, 146.73, 144.78, 143.43, 143.40, 142.76, 141.96, 141.54, 141.50, 139.87, 139.86, 139.82, 139.81, 139.79, 139.57, 139.48, 139.46, 138.08, 137.20, 136.76, 136.43, 136.20, 134.28, 132.17, 130.49, 129.82, 129.78, 129.75, 129.60, 129.22, 129.14, 129.12, 127.25, 127.17, 127.12, 126.00, 122.51, 118.49, 90.76, 89.76, 89.75, 89.73, 78.43, 74.85, 73.74, 73.72, 73.66, 73.64, 71.10, 69.67, 69.44, 69.29, 68.15, 66.63, 63.40, 63.24, 63.08, 62.52, 58.04, 58.00, 56.11, 55.89, 55.83, 55.79, 54.19, 53.30, 50.43, 50.29, 50.19, 36.92, 33.47, 32.35, 32.17, 30.68. [M+H]⁺=1166.6/1168.6; Analytical HPLC: t_{R}=9.81 min

### Example W

### Synthesis of Pentapeptide Helix Mimetic A: Alloc and Allyl deprotection of 208a/208b to Give (S)-2-((11S,14S)-14-amino-9-(2-tert-butoxy-2-oxoethyl)-1-(9H-fluoren-9-yl)-11-isobutyl-16-methyl-3,10,13-trioxo-2-oxa-4,9,12-triazaheptadecan-6-ylamino)-3-(4-((2-bromobenzyloxy)carbonyloxy)-phenyl)propanoic acid (209a, 209b)

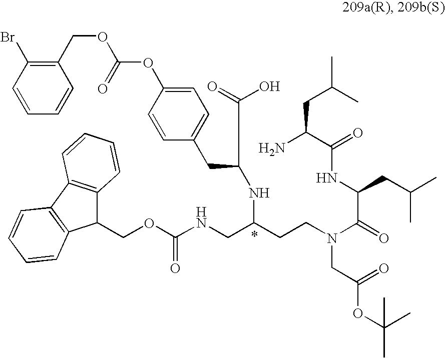

A solution of 208a and 208b (1.7:1 208a(R):208b(S)) (700 mg, 0.67 mmol), barbituric acid (210 mg, 1.34 mmol), palladium-tetrakis(triphenylphosphine) (39 mg, 0.03 mmol), in dichloromethane (20 mL) was stirred under vacuum for 1 h. The solvents were removed under reduced pressure to give a yellow solid, which was used immediately in the following cyclisation reaction. HPLC of this material showed two peaks (6.0 and 6.4 min) corresponding to deprotection side-products (e.g. triphenylphosphine oxide) and one peak (7.9 min, [M+H]⁺=1042/1044) corresponding to both diastereoisomers 209a/209b. The yield of this reaction was assumed as quantitative. Analytical HPLC: t_{R}=7.97 min

### Example X

### Synthesis of Pentapeptide Helix Mimetic A: Intramolecular Amidation of 209a/209b to Form Protected 13-Membered Helix Mimetic: tert-butyl 2-((3S,6S,9S)-11-((((9H-fluoren-9-yl)methoxy)carbonylamino)methyl)-9-(4-((2-bromobenzyloxy)carbonyloxy)benzyl)-3,6-diisobutyl-2,5,8-trioxo-1,4,7,10-tetraazacyclotridecan-1-yl)acetate (210a, 210b)

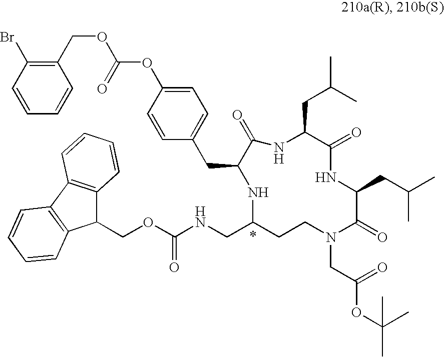

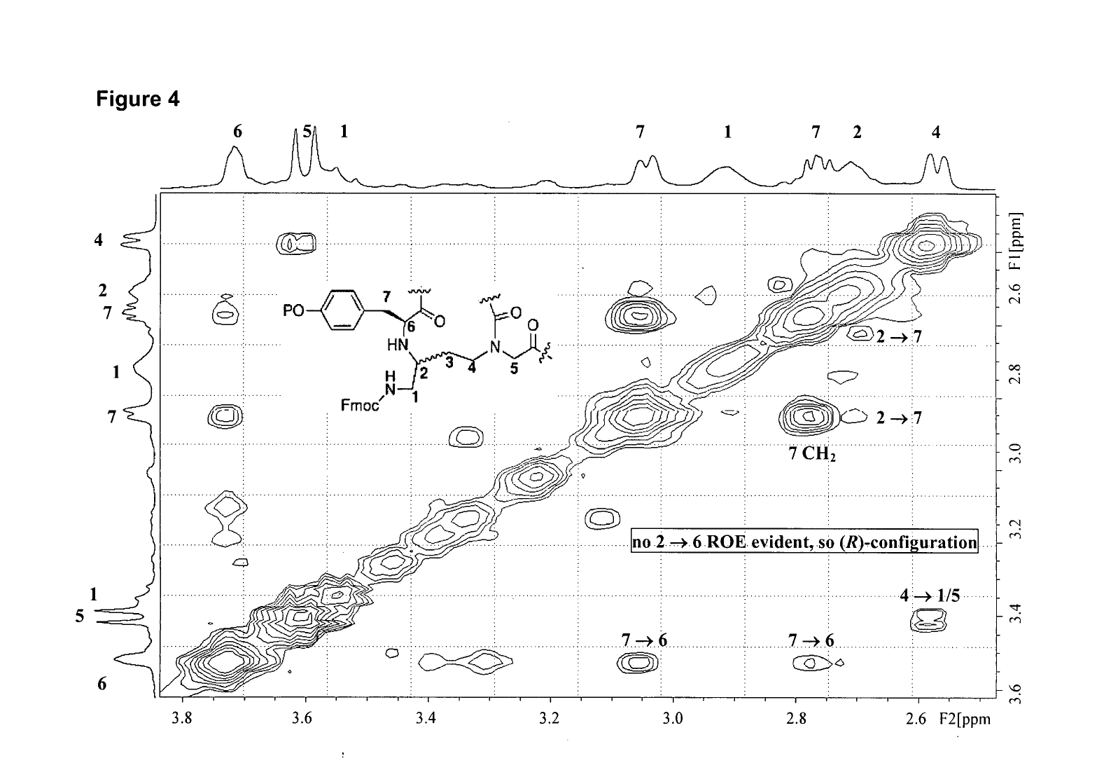

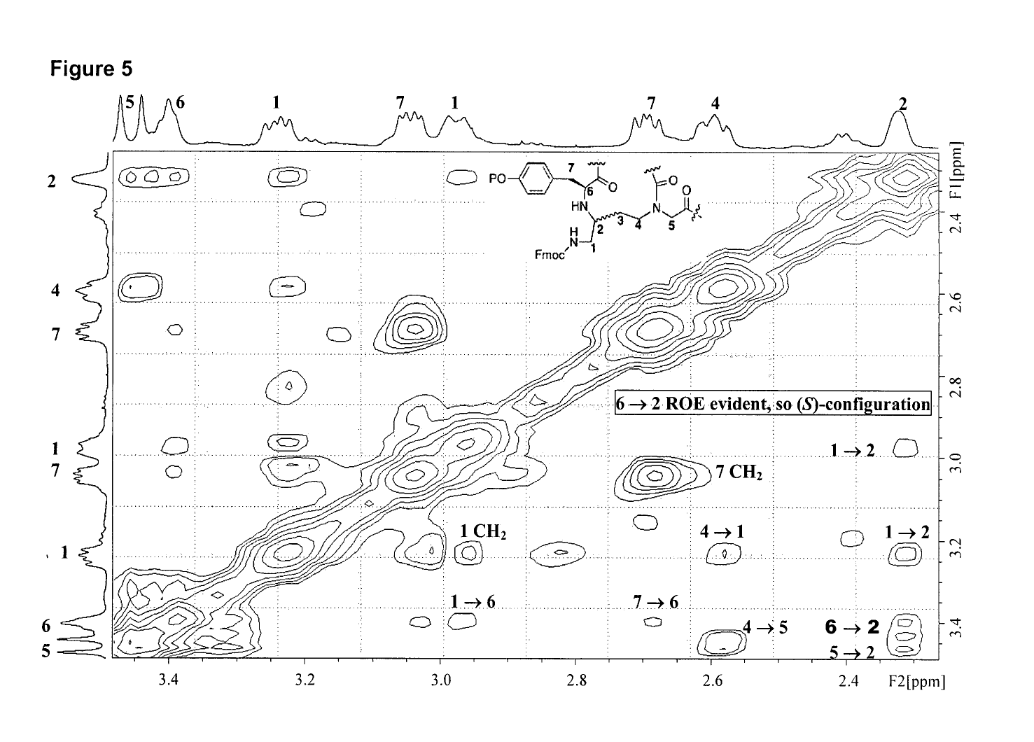

To a vigorously stirred solution of BOP (356 mg, 0.80 mmol), DIEA (121 mg, 0.94 mmol) in DMF (88 mL) was added via syringe pump a solution of the acyclic starting material 209a/209b (crude material from alloc/allyl deprotection, assume 0.67 mmol) in DMF (44 mL). The syringe pump was set at a rate of 0.18 mL/min and addition was complete after 4 h. The rate of the syringe pump addition was checked prior to the reaction, and the activity of BOP/DIEA that were pre-combined was shown to be viable over the 4 h time frame (Initially the cyclisation was carried out on a small test scale in very dilute conditions, and when more concentrated conditions were used a large amount of dimer was formed. Dichloromethane was initially used as a solvent but DMF gave reduced dimer formation, perhaps as more aggregation occurred when using dichloromethane thus giving pseudo-concentrated effect). After syringe pump addition was complete, the DMF was removed under reduced pressure to give the crude cyclised product (1.429 g) as a yellow oil. HPLC analysis of this material showed the same two alloc/allyl deprotection side-products peaks from the previous step (6.0 min, 6.4 min) and two other peaks. A combination of small-scale preparative TLC and small scale preparative HPLC showed that one of these peaks (9.4 min) corresponds to the two 13-membered diastereoisomers and the other peak (10.4 min) to dimeric products. The R and S diastereoiomers could be resolved by TLC using 6:1 ethyl acetate:petroleum ether. The crude cyclisation residue (680 mg) was purified by silica gel flash column chromatography (2:1 to 6:1, ethyl acetate:petroleum spirit, step-wise gradient) several times to give 210a (R isomer, 132 mg) and 210b (S isomer, 102 mg) both as white solids (product masses given are of pure samples, other material recovered as a mixture of R, S, and/or dimers). Both isomers had the same mass spectrum. Assignment of configuration was made using NMR ROESY spectra. The ROESY spectra of 210b (FIG. 5 S isomer) showed a cross peak to the αH of Tyr (2 bonds away) but not to the Tyr βCH₂ (3 bond distance), while the ROESY spectra of 210a (FIG. 4 R isomer) showed no cross peak to the αH of Tyr (2 bonds away) but did show a cross peak to the Tyr βCH₂ (3 bond distance).

### 210a (R)-isomer: (tert-butyl 2-((3S,6S,9S,11R)-1-((((9H-fluoren-9-yl)methoxy)carbonylamino)methyl)-9-(4-((2-bromobenzyloxy)carbonyloxy)-benzyl)-3,6-diisobutyl-2,5,8-trioxo-1,4,7,10-tetraazacyclotridecan-1-yl)acetate)

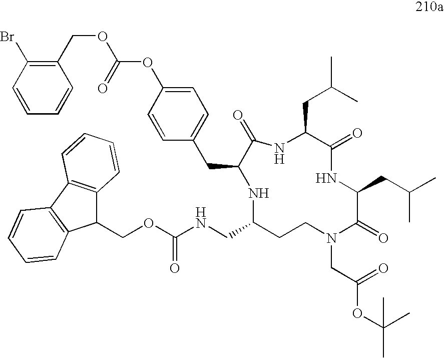

¹H NMR (CDCl₃, 600 MHz) δ 7.77-7.12 (16H, ArH), 6.91 (br s, 1H, NH (Leu)), 6.52 (br s, 1H, NH (Leu)), 5.48 (br s, 1H, NHCH₂), 5.38 (s, 2H, OCH₂ (2BrZ)), 4.19-4.16 (m, 7H, NCHHCO, CH₂ (Fmoc), OCH (Fmoc), NHCH (Leu), NHCH (Leu), NCHHCH₂), 3.72 (br s, 1H, NHCH (tyr)), 3.61 (d, J=18.7 Hz, 1H, NCHHCO), 3.55 (m, 1H, NHCHH), 3.04 (m, 1H, CHH (tyr)), 2.92 (m, 1H, NHCHH), 2.76 (m, 1H, CHH (tyr)), 2.71 (br s, 1H, CH ‘R’ centre), 2.57 (m, 1H, NCHHCH₂), 1.46 (s, 9H, O(CH₃)₃), 1.26-1.84 (m, 8H), 0.77-0.94 (m, 12H, CH₃ Leu); ¹³C NMR (150.9 MHz, CDCl₃) δ 168.60, 153.36, 150.13, 144.11, 143.61, 141.29, 134.84, 134.16, 133.34, 132.96, 130.22, 130.13, 127.75, 127.63, 127.10, 127.07, 125.24, 125.09, 121.23, 120.03, 82.66, 69.66, 67.16, 61.06, 54.23, 48.65, 48.23, 47.24, 47.04, 45.29, 41.25, 39.67, 39.30, 38.57, 29.69, 28.00, 24.86, 24.69, 22.66, 22.43, 22.24.

ROESY specta: see FIG. 4

[M+H]⁺=1024.5/1026.5; Analytical HPLC: t_{R}=9.594 min

### 210b (S)-isomer: (tert-butyl 2-((3S,6S,9S,11S)-11-((((9H-fluoren-9-yl)methoxy)carbonylamino)methyl)-9-(4-((2-bromobenzyloxy)carbonyloxy)-benzyl)-3,6-diisobutyl-2,5,8-trioxo-1,4,7,10-tetraazacyclotridecan-1-yl)acetate)

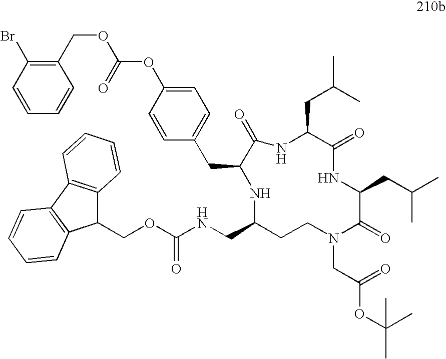

¹H NMR (CDCl₃, 600 MHz) δ 7.75-7.08 (18H, ArH (16H), NH (2H) (Leu)), 5.25 (s, 2H, OCH₂ (2BrZ)), 4.84 (d, J=18.6 Hz, 1H, NCHHCO), 4.76 (m, 1H, NHCH, (Leu)), 4.55 (br s, 1H, NHCH₂), 4.43 (m, 1H, OCH (Fmoc)), 4.21-4.32 (m, 4H, NCHHCH₂, NHCH (Leu), CH₂ (Fmoc)), 3.46 (d, J=18.7 Hz, 1H, NCHHCO), 3.40 (br s, 1H, NHCH (tyr)), 3.24 (m, 1H, NHCHH), 3.05 (m, 1H, CHH (tyr)), 2.99 (m, 1H, NHCHH), 2.70 (m, 1H, CHH (tyr)), 2.60 (m, 1H, NCHHCH₂), 2.32 (br s, 1H, CH ‘S’ centre), 1.44 (s, 9H, O(CH₃)₃), 1.22-1.80 (m, 8H), 0.81-0.97 (m, 12H, CH₃ Leu); ¹³C NMR (150.9 MHz, CDCl₃) δ 173.39, 171.77, 168.45, 157.07, 153.32, 150.05, 143.97, 143.80, 141.27, 135.74, 134.10, 132.88, 130.13, 130.10, 129.96, 127.71, 127.57, 127.08, 127.02, 124.98, 121.24, 119.97, 82.78, 69.73, 66.95, 62.16, 51.36, 50.20, 47.30, 47.26, 46.94, 40.89, 39.43, 38.73, 38.35, 30.59, 29.70, 27.97, 25.07, 24.51, 23.15, 22.70, 22.25.

ROESY specta: see FIG. 5

[M+H]⁺=1024.5/1026.5; Analytical HPLC: t_{R}=9.585 min

### Example Y

### Synthesis of Pentapeptide Helix Mimetic A: t-Butyl Ester Deprotection of 210a to Give 2-((3S,6S,9S,11R)-11-((((9H-fluoren-9-yl)methoxy)-carbonylamino)methyl)-9-(4-((2-bromobenzyloxy)carbonyloxy)benzyl)-3,6-diisobutyl-2,5,8-trioxo-1,4,7,10-tetraazacyclotridecan-1-yl)acetic acid (211a)

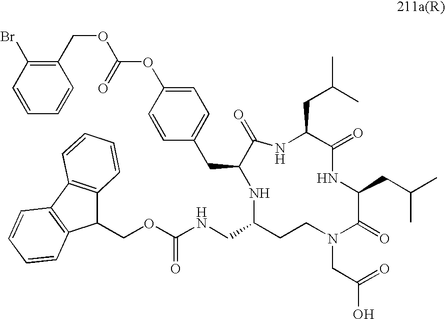

To a solution of 210a(R) (132 mg, 0.13 mmol) in dichloromethane (2 mL) was added TFA (2 mL), and the solution stirred for 3 h at rt. The volatiles were removed under reduced pressure to give an off white solid. This residue was purified by silica gel flash column chromatography (ethyl acetate/0.05% acetic acid to 3:1 ethyl acetate:methanol/0.05% acetic acid, step-wise gradient) to give 211a(R) (83 mg, 0.09 mmol) as a white solid. [M+H]⁺=968.3/970.3, Analytical HPLC: t_{R}=8.533 min

### Example Z

### Synthesis of Pentapeptide Helix Mimetic A: t-Butyl Ester Deprotection of 210b to Give 2-((3S,6S,9S,11S)-11-((((9H-fluoren-9-yl)methoxy)-carbonylamino)methyl)-9-(4-((2-bromobenzyloxy)carbonyloxy)benzyl)-3,6-diisobutyl-2,5,8-trioxo-1,4,7,10-tetraazacyclotridecan-1-yl)acetic acid (211b).

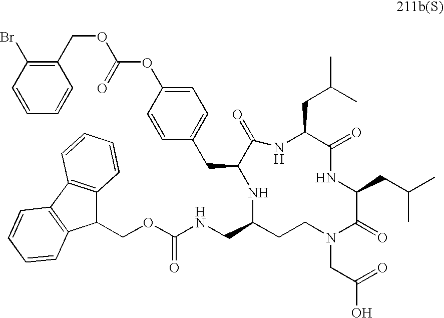

To a solution of 210b(S) (102 mg, 0.10 mmol) in dichloromethane (2 mL) was added TFA (2 mL), and the solution stirred for 3 h at rt. The volatiles were removed under reduced pressure to give an off white solid. This residue was purified by silica gel flash column chromatography (ethyl acetate/0.05% acetic acid to 3:1 ethyl acetate:methanol/0.05% acetic acid, step-wise gradient) to give 211b(S) (87 mg, 0.09 mmol) as a white solid. [M+H]⁺=968.3/970.3. Analytical HPLC: t_{R}=8.361 min

### Example AA

### Synthesis of Pentapeptide Helix Mimetic A: Formation of Methyl Ester of 211a to Give Methyl 2-((3S,6S,9S,11R)-11-((((9H-fluoren-9-yl)methoxy)-carbonylamino)methyl)-9-(4-((2-bromobenzyloxy)carbonyloxy)benzyl)-3,6-diisobutyl-2,5,8-trioxo-1,4,7,10-tetraazacyclotridecan-1-yl)acetate (212a)

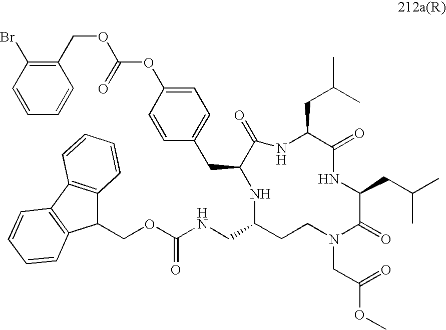

To a solution of 211a (48 mg, 0.05 mmol, approx 9:1 mixture of R pentapeptide 211a and dimers) in methanol (5 mL) was added EDC (10.5 mg, 0.06 mmol) and DMAP (1 mg), and the mixture stirred overnight at rt. The volatiles were removed under reduced pressure, and the residue was dissolved in ethyl acetate:water (1:1, v:v, 100 mL). The organics were washed two times with saturated aqueous sodium bicarbonate, dried over magnesium sulfate, and the evaporated under reduced pressure to give a yellow oil (28 mg). HPLC and MS analysis of this crude product 212a showed some loss of the tyrosine 2BrZ protecting group. The product was used without further purification in the next reaction. Analytical HPLC (0-40% B over 40 min, 1 mL/min, 214 nm): t_{R}=6.26 min

### Example AB

### Synthesis of Pentapeptide Helix Mimetic A: Formation of Methyl Ester of 211b to Give Methyl 2-((3S,6S,9S,11S)-11-((((9H-fluoren-9-yl)methoxy)-carbonylamino)methyl)-9-(4-((2-bromobenzyloxy)carbonyloxy)benzyl)-3,6-diisobutyl-2,5,8-trioxo-1,4,7,10-tetraazacyclotridecan-1-yl)acetate (212b)

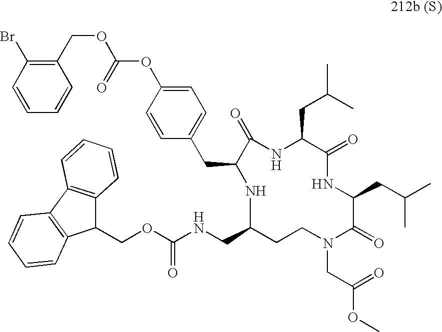

To a solution of 211b (40 mg, 0.04 mmol, approx 8:1 mixture of S pentapeptide 211b and dimers) in methanol (5 mL) was added EDC (9 mg, 0.044 mmol) and DMAP (1 mg), and the mixture stirred overnight at rt. The volatiles were removed under reduced pressure, and the residue was dissolved in ethyl acetate:water (1:1, v:v, 100 mL). The organics were washed two times with saturated aqueous sodium bicarbonate, dried over magnesium sulfate, and the evaporated under reduced pressure to give a yellow oil (37 mg). HPLC and MS analysis of this crude product 212b showed some loss of the tyrosine 2BrZ protecting group. The product was used without further purification in the next reaction.

### Example AC

### Synthesis of Pentapeptide Helix Mimetic A: Cleavage of Fmoc and 2BrZ Protecting Groups of 211a to Give Methyl 2-((3S,6S,9S,1R)-11-(aminomethyl)-9-(4-hydroxybenzyl)-3,6-diisobutyl-2,5,8-trioxo-1,4,7,10-tetraazacyclotridecan-1-yl)acetate (213a)

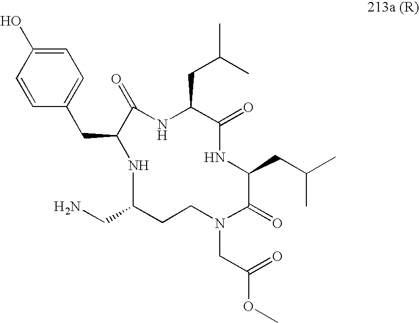

To a solution of the starting material 212a (28 mg) in dichloromethane (1 mL) was added piperidine (0.2 mL) and the mixture stirred at rt for 3 h. The volatiles were removed under reduced pressure, and the residue dissolved in dichloromethane and evaporated under reduced pressure consecutively three times. The residue was then dissolved in buffer A and B (1:1, v:v) and lypholised to give a white solid (25 mg). This material was purified by semi-preparative HPLC to give the product 213a (1.7 mg) as a white solid. Other material containing impurities was also isolated following HPLC. [M+H]⁺=548.2; Analytical HPLC: t_{R}=29.79 min ¹H NMR spectra, 9:1H₂O:D₂O. Consistent with structure. Variable temperature ¹H NMR were run at 5° C., 15° C., 25° C., and 35° C.

### Example AD

### Synthesis of Pentapeptide Helix Mimetic A: Cleavage of Fmoc and 2BrZ Protecting Groups of 212b to Give Methyl 2-((3S,6S,9S,11S)-11-(aminomethyl)-9-(4-hydroxybenzyl)-3,6-d iisobutyl-2,5,8-trioxo-1,4,7,10-tetraazacyclotridecan-1-yl)acetate (213b)

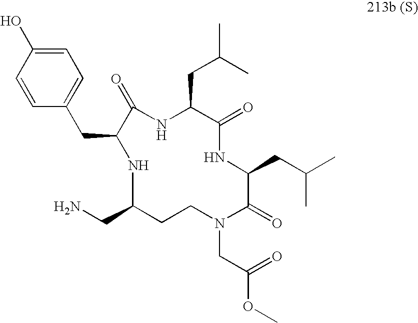

To a solution of the starting material 212b (37 mg) in dichloromethane (1 mL) was added piperidine (0.2 mL) and the mixture stirred at rt for 3 h. The volatiles were removed under reduced pressure, and the residue dissolved in dichloromethane and evaporated under reduced pressure consecutively three times. The residue was then dissolved in buffer A and B (1:1, v:v) and lypholised to give a white solid (35 mg). This material was purified by semi-preparative HPLC to give the product 213b (1 mg) as a white solid. Other material containing impurities was also isolated following HPLC. [M+H]⁺=548.5; Analytical HPLC: t_{R}=29.94 min; ¹H NMR spectra, 9:1H₂O:D₂O. Consistent with structure. Variable temperature ¹H NMR were run at 5° C., 15° C., 25° C., and 35° C.

### Example AE

### Synthesis of Pentapeptide Helix Mimetic B: Weinreb Amide Formation to Give tert-Butyl 2-(methoxy(methyl)amino)-2-oxoethylcarbamate (214)

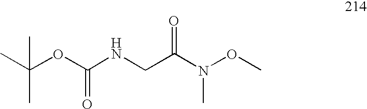

A solution of Boc-Gly-OH (25 g, 140 mmol), N,O-dimethylhydroxylamine (18.1 g, 180 mmol), HBTU (64.4 g, 170 mmol), DIEA (59 mL, 340 mmol) and DMF (100 mL) was stirred at rt overnight. The solvent was removed under reduced pressure, and the residue dissolved in 1/1 v:v ethyl acetate:1M aqueous HCl. The organic layer was washed with 1M aqueous HCl, saturated aqueous sodium bicarbonate, then saturated brine sequentially three times, dried over magnesium sulfate, filtered, and evaporated under reduced pressure to give the Weinreb amide 214 (18.65 g, 60%) as a white solid.

### Example AF

### Synthesis of Pentapeptide Helix Mimetic B: Vinyl Grignard Reaction to Form tert-Butyl 2-oxobut-3-enylcarbamate (215)

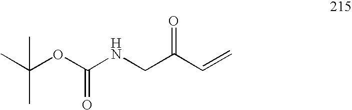

To a solution of Boc-Gly-Weinreb amide 214 (10.17 g, 46.63 mmol) in THF at 0° C. was added vinyl magnesium bromide (140 mL of a 1M solution, 140 mmol, freshly prepared) in two portions over one hour. The solution was allowed to warm to room temperature, stirred for a further 2 h, then quenched by addition of 1M aqueous HCl/ice. The aqueous layer was extracted three times with dichloromethane, dried over magnesium sulfate, filtered, and the solvent removed under reduced pressure to give 215 (8.37 g, 97%) as a yellow oil. To prevent polymerisation, 215 was stored at 0° C. as a 0.3 M solution in dichloromethane. ¹H NMR (400 MHz, CDCl₃) δ 6.15 (m, 2H, CH═CH₂), 5.71 (dd, J=1.2, 10.1 Hz, 1H, CH═CH₂), 5.44 (br s, 1H, NH), 4.02 (d, J=4.9 Hz, 2H, NHCH₂), 1.23 (s, 9H, C(CH₃)₃). ¹³C NMR (160 MHz, CDCl₃) δ 194.64, 155.32, 133.15, 128.95, 79.05, 47.90, 27.82

Analytical HPLC: t_{R}=4.78 min

### Example AG

### Synthesis of Pentapeptide Helix Mimetic B: Conjugate Addition of Leu-OtBu to Form (S)-tert-Butyl 2-(4-(tert-butoxycarbonylamino)-3-oxobutylamino)-4-methylpentanoate (216)

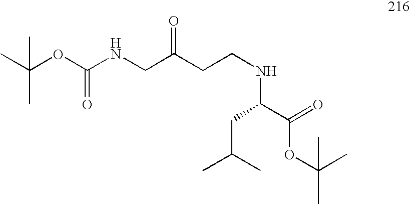

To a solution of Leu-^{t}Bu ester, HCl (5.37 g, 24.0 mmol), DIEA (9.2 mL, 52.8 mmol), and dichloromethane (40 mL) was added a solution of tert-butyl 2-oxobut-3-enylcarbamate 215 in dichloromethane (89 mL of a 0.3 mmol/mL solution, 26.58 mmol). The reaction was checked by mass spectrometry after stirring at rt for 20 min, and showed the product peak of 216 at [M+H]⁺=373. This solution was used without purification. [M+H]⁺=373.3

### Example AH

### Synthesis of Pentapeptide Helix Mimetic B: Acylation of 216 with Cbz-Gly-OH to Give (S)-tert-Butyl 2-(2-(benzyloxycarbonylamino)-N-(4-(tert-butoxycarbonylamino)-3-oxobutyl)-acetamido)-4-methylpentanoate (217)

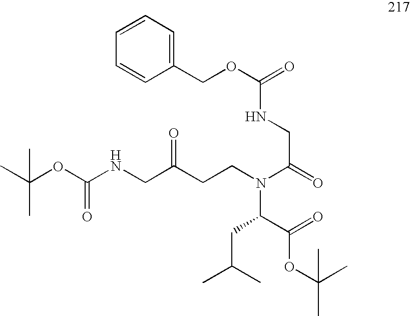

To the crude solution of 216 (assumed 100% yield, 24 mmol) was added Cbz-Gly-OH (10.10 g, 48.3 mmol), HATU (18.63 g, 48.3 mmol), and DIEA (15.8 mL, 96.6 mmol), and the mixture stirred at rt overnight. The solvent was removed under reduced pressure and the residue dissolved in ethyl acetate/water (1:1, v:v). The aqueous layer was extracted twice with ethyl acetate, the organic layers combined, and then washed with saturated aqueous sodium bicarbonate, saturated ammonium chloride, and saturated brine, dried over magnesium sulfate, filtered, and the solvent evaporated under reduced pressure. The residue was purified by silica gel flash column chromatography (3:2 to 2:3, petroleum spirit:ethyl acetate, step-wise gradient) to give 217 (10.50 g, 77%) as a yellow oil. ¹H NMR (600 MHz, (CD₃)₂SO, 60° C., intergrals correspond to major rotomer) δ 7.33 (m, 5H, ArH), 7.10 (m, 1H NH), 6.89 (m, 1H NH), 5.04 (s, 2H, OCH₂Ar), 4.31 (m, NCH Leu, minor rotomer), 4.24 (m, 1H, NCH Leu), 3.89 (m, 2H, NHCH₂), 3.76-3.61 (m, 2H, NHCH₂), 3.46-3.16 (m, 2H, COCH₂CH₂, multiple rotamers), 2.81-2.59 (m, 2H, COCH₂CH₂ multiple rotamers), 1.73 (m, 1H, CHCHH Leu), 1.61 (m, 1H, CHCHH Leu), 1.50 (m, 1H, CH₂CH Leu), 1.39 (br s, 18H, C(CH₃)₃), 0.89 (m, 6H, CH₃Leu); ¹³C NMR (160 MHz, (CD₃)₂SO, 60° C., major rotomer) δ 206.06, 170.03, 168.80, 156.44, 155.75, 137.13, 128.29, 127.72, 127.63, 80.37, 78.15, 35.38, 35.29, 58.14, 49.84, 41.92, 37.68, 28.14, 27.51, 24.44, 23.02, 21.93. ¹H NMR compared at 25° C. and 60° C., showing minimisation but not elimination of rotamers at the higher temperature. [M+H]⁺=564.3; Analytical HPLC: t_{R}=8.99 min

### Example AI

### Synthesis of Pentapeptide Helix Mimetic B: Cbz Deprotection of 217 to Give (S)-tert-butyl 2-(2-amino-N-(4-(tert-butoxycarbonylamino)-3-oxobutyl)acetamido)-4-methylpentanoate (218)

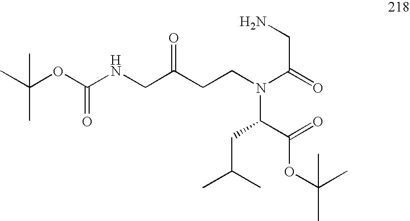

To a solution of 217 (2.92 g, 5.18 mmol) in methanol (200 mL) and water (70 mL) was added 1M aqueous HCl (5.7 mL, 5.70 mmol) and 10% Pd/C (300 mg), and the solution stirred for 2 h under an atmosphere of hydrogen (hydrogen balloon). The solvent was removed under reduced pressure to give the Cbz-deprotected amine 218 hydrochloride salt (2.15 g, 4.61 mmol, 89%) as a yellow solid. This material was used without purification in the subsequent reaction. [M+H]⁺=430.3. A peak corresponding to the imine [M+H]⁺=412.3 was also observed in the mass spectrum. Analytical HPLC: t_{R}=6.14 min

### Example AJ

### Synthesis of Pentapeptide Helix Mimetic B: Synthesis of Alloc-Ser(Bzl)-OH (219)

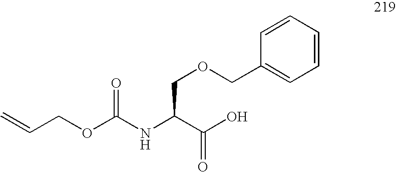

To a solution of Boc-Ser(Bzl)-OH (10.62 g, 36 mmol) in dichloromethane (10 mL) was added TFA (5 mL) and the mixture stirred for 1 h at rt. The volatiles were removed under reduced pressure, and the residue dissolved in acetonitrile (20 mL) and water (20 mL). Saturated aqueous sodium bicarbonate was added until the pH reached 8 to 9, the mixture was cooled to 0° C., allyl chloroformate (4.21 mL, 39.6 mmol) was added dropwise and the resulting solution stirred for 2 h at rt. Water (20 mL) was added and the solution was acidified to pH 2 using 1M aqueous hydrochloric acid, and extracted twice with ethyl acetate. The organics were combined, dried over magnesium sulfate, filtered, and the solvents removed under reduced pressure. The residue was purified by silica gel flash column chromatography (95:5, ethyl acetate:methanol) to give the N-Alloc-Ser(Bzl)-OH 219 (9.31 g, 93%) as a yellow oil. ¹H NMR (400 MHz, CDCl₃) δ 8.11 (br s, 1H, CO₂H), 7.32 (m, 5H, ArH), 6.01 (d, J=Hz, 1H, NH), 5.89 (m, 1H, ═CH), 5.33 (m, 1H, CH═CHH), 5.19 (m, 1H, CH═CHH), 4.57 (m, 2H, OCH₂ alloc), 4.51 (s, 2H, OCH₂Ar), 3.92 (m, 1H, CHH βSer), 3.72 (m, 1H, CHH βSer). ¹³C NMR (160 MHz, CDCl₃) δ 173.35, 156.07, 137.00, 132.15, 128.11, 128.11, 127.53, 127.39, 117.55, 72.95, 69.29, 65.76, 53.91 [M+H]⁺=280.1Analytical HPLC: t_{R}=5.99 min

### Example AK

### Synthesis of Pentapeptide Helix Mimetic B: Acylation With Alloc-Ser(Z)-OH (7S,13S)-tert-butyl 7-(benzyloxymethyl)-12-(4-(tert-butoxycarbonylamino)-3-oxobutyl)-15-methyl-5,8,11-trioxo-4-oxa-6,9,12-triazahexadec-1-ene-13-carboxylate (220)

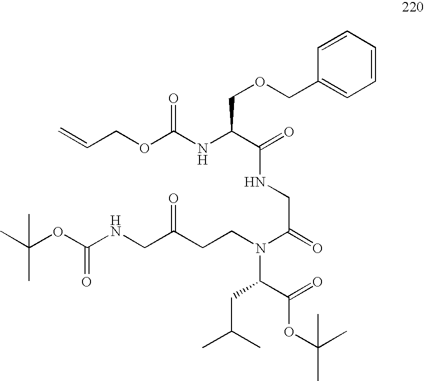

To a solution of Cbz-deprotected amine hydrochloride salt 218 (2.15 g, 4.61 mmol) in DMF (100 mL) was added Alloc-Ser(Bzl)-OH 219 (1.74 g, 6.25 mmol), HATU (2.38 g, 6.25 mmol), and DIEA (4.36 mL, 25 mmol), and the resulting solution stirred overnight at rt. Ethyl acetate/water (1:1, v:v) was added and the aqueous layer extracted twice with ethyl acetate. The organic layers were combined, then washed with saturated aqueous sodium bicarbonate, saturated ammonium chloride, and saturated brine, dried over magnesium sulfate, filtered, and the solvent evaporated under reduced pressure. The residue was purified by silica gel flash column chromatography (1:1 to 3:2, ethyl acetate: petroleum spirit, step-wise gradient) to give the N-terminal Boc-protected analgoue of 220 (1.97 g, 62%) as a pale yellow oil.

¹H NMR (400 MHz, CDCl₃) δ 7.41 (br s, 1H, NH N-term gly), 7.31 (m, 5H, ArH), 5.91 (m, 1H, CH═CH₂), 5.64 (br s, 1H, NH Ser), 5.31 (m, 1H, CH═CHH), 5.20 (m, 2H, CH═CHH, NH gly), 4.57 (m, 4H, OCH₂ alloc, OCH₂Ar), 4.41 (m, 1H, CH Ser), 4.15-3.98 (m, 5H, NHCH Leu, NHCH₂), 3.59 (m, 1H, OCHH Ser), 3.59 (m, 3H, OCHH Ser, COCH₂), 2.89-2.66 (m, 2H, NCH₂), 1.80-1.49 (m, 3H, CH₂CH Leu), 1.44 (s, 9H, C(CH₃)₃), 1.41 (s, 9H, C(CH₃)₃), 0.93 (m, 6H, CH₃ Leu); ¹³C NMR (150.9 MHz, (CD₃)₂SO, 25° C., major rotomer) δ 206.06, 169.95, 169.58, 168.13, 155.80, 155.75, 138.17, 133.49, 128.17, 127.50, 127.39, 117.03, 80.42, 78.16, 71.97, 69.86, 64.55, 58.14, 54.80, 49.82, 40.55, 38.36, 37.63, 28.15, 27.54, 24.43, 23.43, 21.93; [M+H]⁺=691.3; Analytical HPLC: t_{R}=9.15 min

### Example AL

### Synthesis of Pentapeptide Helix Mimetic B: N-Boc cleavage, Fmoc protection of 220 to Give (7S,13S)-tert-butyl 12-(4-(((9H-fluoren-9-yl)methoxy)carbonylamino)-3-oxobutyl)-7-(benzyloxymethyl)-15-methyl-5,8,11-trioxo-4-oxa-6,9,12-triazahexadec-1-ene-13-carboxylate (221)

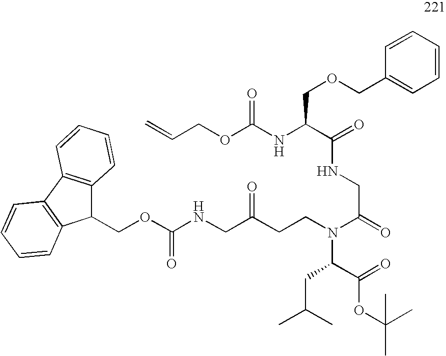

To the Boc-protected analogue 220 (1.64 g, 2.37 mmol) in dichloromethane (28 mL) at 0° C. was added TFA (22 mL) and the mixture stirred for 30 mins. The solvents were evaporated under reduced pressure making sure the water bath remained cold. The yellow residue was used immediately in the next reaction. MS and HPLC of this residue showed the Boc group cleaved and the ^{t}Bu ester intact. To a solution of the crude Boc-deprotected material (2.37 mmol, assume 100%) in dioxane (150 mL) and water (150 mL) was added Fmoc-succinimide (1.6 g, 4.74 mmol) and DIEA (1.6 mL, 9.80 mmol), and the solution stirred at rt overnight. Water was added and the aqueous layer was extracted twice with ethyl acetate. The combined organic layers were washed with saturated aqueous sodium bicarbonate, dried with magnesium sulfate, filtered, and the solvent removed under reduced pressure. The residue was purified by silica gel flash column chromatography (1:1, petroleum ether:ethyl acetate) to give 221 as a white foam (1.561 g, 81% from Boc material). [M+H]⁺=813.1; Analytical HPLC: t_{R}=9.98 min

### Example AM

### Synthesis of Pentapeptide Helix Mimetic B: Synthesis of N-Boc-Leu-O-allyl ester (222)

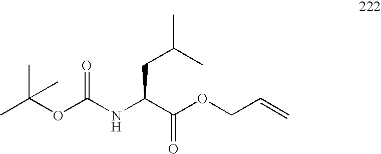

A solution of N-Boc-Leu-OH (3.003 g, 13 mmol), allyl alcohol (754 mg, 13 mmol), DMAP (16 mg, 0.13 mmol), and DIC (2.2 mL, 14.2 mmol) in dichloromethane (30 mL) was stirred at 0° C. for 2 h then overnight at rt. The solvent was evaporated, dissolved in ethyl acetate/H₂O (1:1 v:v), and the organic layer separated and washed with saturated aqueous sodium bicarbonate, water, dried with magnesium sulfate, filtered, and the solvent removed under reduced pressure. The residue was purified by silica gel flash column chromatography (5:1, petroleum ether:ethyl acetate) to give N-Boc-Leu-O-allyl ester 222 (3.233 g, 11.93 mmol, 92%) as a colourless oil. ¹H NMR (400 MHz, CDCl₃) δ 5.89 (m, 1H, CH═CH₂), 5.34-5.22 (m, 2H, CH═CH₂), 4.90 (m, 1H, NH), 4.61 (m, 2H, OCH₂CH), 4.32 (m, 1H, NHCH), 1.74-1.46 (m, 3H, Leu CH CH₂), 1.42 (s, 9H, C(CH₃)₃), 0.94 (m, 6H, CH₃)¹³C NMR (150.9 MHz, CDCl₃) δ 173.14, 153.37, 131.68, 118.48, 79.74, 65.64, 52.09, 41.76, 28.26, 24.73, 22.79, 21.82 [M+H]⁺=272.1 Analytical HPLC: t_{R}=7.81 min

### Example AN

### Synthesis of Pentapeptide Helix Mimetic B: Synthesis of H-Leu-O-allyl ester (223)

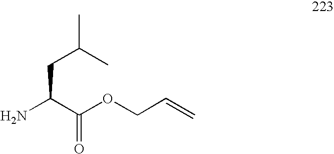

To a solution of N-Boc-Leu-O-allyl ester 222 (1.878 g, 6.93 mmol) in dichloromethane (6 mL) was added TFA (2 mL). The solution was stirred for 30 min at rt, at which stage mass spectrometry and HPLC analysis showed only the desired Boc-deprotected material. The volatiles were removed under reduced pressure, and the residue was dissolved in dichloromethane. To neutralise the TFA salt the organic layer was washed twice with saturated sodium bicarbonate, dried using magnesium sulfate, and then the solvent removed under reduced pressure to give the free amine H-Leu-O-allyl ester 223 (885 mg, 87%) as a yellow oil.

[M+H]⁺=171

### Example AO

### Synthesis of Pentapeptide Helix Mimetic B: Reductive Amination of 221 with Leu-O-allyl ester 223 to Give (2S,12S)-allyl 4-((((9H-fluoren-9-yl)methoxy)carbonylamino)methyl)-12-(benzyloxymethyl)-7-((S)-1-tert-butoxy-4-methyl-1-oxopentan-2-yl)-2-isobutyl-8,11,14-trioxo-15-oxa-3,7,10,13-tetraazaoctadec-17-en-1-oate (224a,224b)

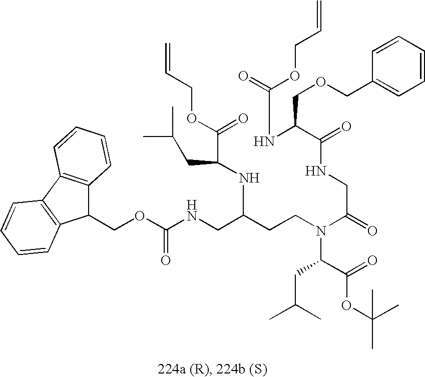

A solution of ketone 221 (1.560 g, 1.92 mmol), Leu-O-allyl ester 223 (657 mg, 3.84 mmol), sodium triacetoxyborohydride (527 mg, 2.50 mmol), and dichloroethane (5 mL) was stirred overnight at rt. Ethyl acetate was added and the organic layer washed four times with saturated aquesous sodium bicarbonate, dried with magnesium sulfate, filtered, and the solvent removed under reduced pressure. The residue was purified by silica gel flash column chromatography (1:1 to 1:3, petroleum ether:ethyl acetate, step-wise gradient) to give 224a/224b as a mixture of diastereoisomers (1.357 g, 73%) and as a white foam. The 224a/224b mixture was inseparable, and appeared as one spot on TLC and one peak on HPLC. The ¹H and ¹³C NMR spectra showed the diastereoisomers to have very similar chemical shifts, and as such the isomer ratio could not be assigned. The ratio was established after N-alloc and O-ally deprotection in the following step. ¹H NMR (600 MHz, (CD₃)₂SO, 60° C.) δ 7.90 (br s, 1H, NH gly), 7.87 (m, 2H, ArH Fmoc), 7.66 (m, 2H, ArH Fmoc), 7.41 (m, 2H, ArH Fmoc), 7.31 (m, 7H, ArH Fmoc, ArH Cbz), 7.20 (1H, NH Ser), 7.08 (br s, 1H, NHCH₂CH), 5.90 (m, 2H, ═CH), 5.29 (m, 2H, ═CH₂), 5.17 (m, 2H, ═CH₂), 4.56 (m, 2H, OCH₂CH═), 4.49 (m, 4H, OCH₂CH═, OCH₂Ar), 4.36 (m, 1H, NHCH Ser), 4.29 (m, 3H, OCH₂ Fmoc, NCH Leu), 4.21 (m, 1H, CH Fmoc), 4.00 (m, 2H, NHCH₂CO gly), 3.69 (m, 1H, OCHH Ser), 3.63 (m, 1H, OCHH Ser), 3.46-3.31 (m, 3H, NHCH Leu, NCH₂), 3.16-2.99 (m, 2H, NHCH₂CH), 2.57 (m, 2H, NHCH₂C*H), 1.75-1.35 (m, 8H, CH. Leu, CH₂ Leu, NCH₂CH₂), 1.25 (s, 9H, C(CH₃)₃), 0.86 (m, 12H, CH₃ Leu); ¹³C NMR (150.9 MHz, (CD₃)₂SO, 60° C., mixture of diastereoisomers and rotamers) δ 169.23, 167.92, 143.69, 143.64, 140.49, 137.98, 133.20, 132.29, 127.82, 127.28, 127.15, 127.03, 126.72, 124.77, 119.74, 117.65, 116.66, 71.86, 69.78, 65.34, 64.31, 64.20, 57.67, 57.45, 54.77, 54.30, 46.60, 42.58, 40.22, 37.61, 33.44, 31.26, 30.74, 28.65, 28.61, 28.23, 27.30, 24.30, 24.12, 22.52, 22.28, 22.04, 21.98, 21.71; [M+H]⁺=968.0; Analytical HPLC: t_{R}=9.339 min

### Example AP

### Synthesis of Pentapeptide Helix Mimetic B: Alloc and Allyl deprotection of 224a/224b to Give (4S,14S)-12-((((9H-fluoren-9-yl)methoxy)-carbonylamino)methyl)-4-amino-9-((S)-1-tert-butoxy-4-methyl-1-oxopentan-2-yl)-14-isobutyl-5,8-dioxo-1-phenyl-2-oxa-6,9,13-triazapentadecan-15-oic acid (225a,225b).

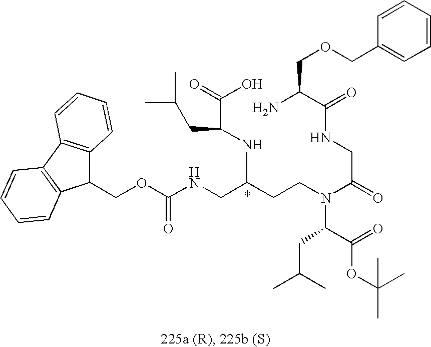

A solution of 224a/224b (1.148 g, 1.187 mmol), barbituric acid (371 mg, 2.37 mmol), palladium-tetrakis(triphenylphosphine) (69 mg, 0.06 mmol), in dichloromethane (20 mL) were stirred under vacuum for 1 h. The solvents were removed under reduced pressure to give a yellow solid, which was used immediately in the following cyclisation reaction. Analytical HPLC of this material showed various peaks. Preparative analytical HPLC and mass spectrometry analysis revealed the 6.0/6.4 min peaks correspond to deprotection side-products (e.g. triphenylphosphone oxide) and the two other peaks (7.2/7.3 min, [M+H]⁺=844) as the desired material 225a/225b. The 7.2/7.3 min HPLC peaks (ratio of 1.8:1 by peak integration) were assumed to be the product diastereoisomers. [M+H]⁺=844.1; Analytical HPLC: t_{R}=7.156, 7.345 min

### Example AQ

### Synthesis of Pentapeptide Helix Mimetic B: Intramolecular Amidation of 225a/225b to Form Protected 13-Membered Helix Mimetic (2S)-tert-butyl 2-((6S,9S)-11-((((9H-fluoren-9-yl)methoxy)carbonylamino)methyl)-6-(benzyloxymethyl)-9-isobutyl-2,5,8-trioxo-1,4,7,10-tetraazacyclotridecan-1-yl)-4-methylpentanoate (226a,226b)

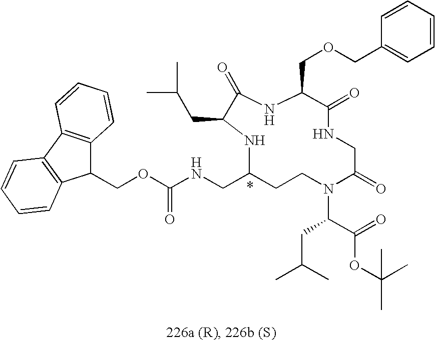

To a vigorously stirred solution of BOP (630 mg, 1.42 mmol), DIEA (214 mg, 1.66 mmol) in DMF (160 mL) was added via syringe pump a solution of the acyclic starting material 225a/225b (1.187 mmol from alloc/allyl deprotection assuming quantitative yield) in DMF (80 mL). The syringe pump was set at a rate of 0.27 mL/min and addition was complete after 5 h (see Helix Mimetic A synthesis for syringe pump optimisation comments). After addition via syringe pump was complete, the DMF was removed under reduced pressure to give the cyclised product (3.4 g, mixture of diastereoisomers) as a yellow oil. The diastereoisomers could be resolved by HPLC and TLC. The residue was purified by silica gel flash column chromatography (1:1 to 6:1, ethyl acetate:petroleum spirit, step-wise gradient) to give 226a)(R isomer, 384 mg) and 226b (S isomer, 419 mg) both as white solids (products are >90% pure, other material recovered as a mixture of R, S, and/or dimers. This>90% purity material was used in the tBu ester deprotection reaction). To assign stereochemistry, a sample of the 226b (S isomer) was analysed using 2D NMR spectroscopy. A sample containing alloc/allyl deprotection side products (6.3 min peak) but no 226b R isomer was used (see analytical HPLC trace below). The ROESY spectra of 226b (S isomer) showed a cross peak to the αH of Leu (2 bonds away) but not to the Leu CH₂ (3 bond distance). [M+H]⁺=826.5. Both isomers have the same mass spectrum; Analytical HPLC: (R) t_{R}=8.373 min; (S) t_{R}=8.417 min

### Example AR

### Synthesis of Pentapeptide Helix Mimetic B: tBu ester deprotection of 226a to Give (S)-2-((6S,9S,11R)-11-((((9H-fluoren-9-yl)methoxy)carbonylamino)-methyl)-6-(benzyloxymethyl)-9-isobutyl-2,5,8-trioxo-1,4,7,10-tetraazacyclo-tridecan-1-yl)-4-methylpentanoic acid (227a)

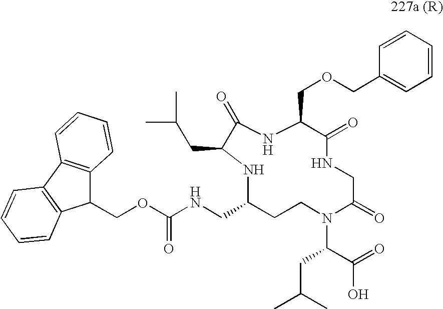

To a solution of 226a(R) (184 mg, 0.22 mmol, >90% pure) in dichloromethane (2 mL) was added TFA (1 mL), and the solution stirred for 1 h at rt. The volatiles were removed under reduced pressure to give an off white solid. This residue was purified by silica gel flash column chromatography (ethyl acetate/0.05% acetic acid to 6:1 ethyl acetate: methanol/0.05% acetic acid, step-wise gradient) to give 227a(R) (104 mg, 0.14 mmol) as a white solid. ¹H NMR (600 MHz, CDCl₃) δ 8.74 (br s, 1H, NH Ser), 7.97 (m, 1H, NHCH₂CO gly), 7.77 (m, 2H, ArH Fmoc), 7.59 (m, 2H, ArH Fmoc), 7.43 (m, 2H, ArH Fmoc), 7.18-7.06 (m, 7H, ArH Fmoc, ArH Bzl), 6.03 (br s, 1H, NHCH₂CH gly), 4.75 (m, 1H, NCHH), 4.52 (m, 1H, NHCHHCO), 4.37 (m, 2H, CH Fmoc, NCH Leu), 4.22 (m, 1H, OCHH Fmoc), 4.20 (m, 2H, NHCHHCO, CH Ser), 4.09 (m, 1H, OCHH Fmoc), 4.03-3.86 (m, 3H, NHCH Leu, OCH₂ Ser), 3.64 (m, 1H, NHCHHCH), 3.43 (m, 1H, NHCHHCH), 3.22 (m, 1H, NHCH₂CH R centre), 2.88 (m, 1H, NCHH), 2.09 (m, 1H, NCH₂CHH), 1.88-1.50 (6H, CH₂ CH Leu), 1.44 (m, 1H, NCH₂CHH), 0.92 (m, 3H, CH₃ Leu), 0.87 (m, 3H, CH₃ Leu), 0.77 (m, 3H, CH₃ Leu), 0.72 (m, 3H, CH₃ Leu). ¹³C NMR (150.9 MHz, CDCl₃) δ 175.68, 172.39, 169.21, 167.81, 160.89, 144.69, 143.19, 141.44, 140.90, 128.07, 127.22, 127.12, 124.93, 119.97, 119.75, 72.54, 68.59, 67.08, 60.08, 57.33, 56.58, 47.02, 42.88, 42.43, 39.87, 38.77, 36.48, 28.57, 27.48, 24.74, 23.99, 23.20, 23.01, 22.51, 22.37. R isomer was assigned by ROE cross peaks. The proton at the reductive amination centre (R isomer stereocentre) showed a cross peak to Leu CH₂ (3 bond distance Leucine) but not Leu α-CH (2 bond distance Leucine) (analogous to Gal R pentapeptide). [M+H]⁺=770.2; Analytical HPLC: t_{R}=7.451 min

### Example AS

### Synthesis of Pentapeptide Helix Mimetic B. tBu ester deprotection fo 226b to Give (S)-2-((6S,9S,11S)-11-((((9H-fluoren-9-yl)methoxy)carbonylamino)-methyl)-6-(benzyloxymethyl)-9-isobutyl-2,5,8-trioxo-1,4,7,10-tetraazacyclo-tridecan-1-yl)-4-methylpentanoic acid (227b)

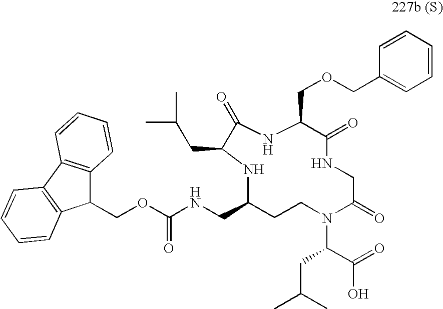

To a solution of 226b(S) (419 mg, 0.51 mmol) in dichloromethane (4 mL) was added TFA (2 mL), and the solution stirred for 1 h at rt. The volatiles were removed under reduced pressure to give an off white solid. This residue was purified by silica gel flash column chromatography (ethyl acetate/0.05% acetic acid to 3:1 ethyl acetate: methanol/0.05% acetic acid, step-wise gradient) to give 227b(S) (86 mg, 0.11 mmol) as a white solid. Other material was also recovered but could not be separated from other impurities. [M+H]⁺=770.2. Analytical HPLC: t_{R}=7.562 min

### Example AT

### Synthesis of Linear Reference Pentapeptide A: SPPS of Ac-GY(tBu)LLG-OH (228)

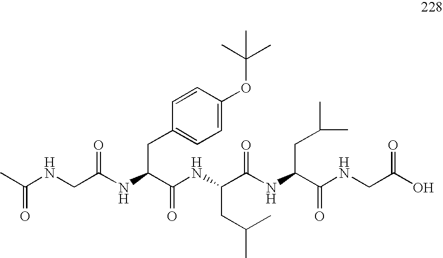

The peptide was assembled on chlorotrityl chloride resin (1.84 g, 1.2 mmol) using standard Fmoc SPPS as outlined in the General Procedures. After coupling of Gly-5 the Fmoc protecing group was cleaved and the N-terminus acetylated, the resin washed and dried, to give the product peptide on resin (3.05 g). To a portion of this resin (1.0 g) in dichloromethane (100 mL) was added TFA (1.5 mL) and the solution stirred at rt for 1 h. The solution was filtered and the volatiles removed by rotary evaporation to give the product peptide 228 (still containing the tyrosine tBu ether protecting group) an off white solid (750 mg)

### Example AU

### Synthesis of Linear Reference Pentapeptide A: Methyl Ester Formation of 228 to give Ac-GY(tBu)LLG-OMe (229)

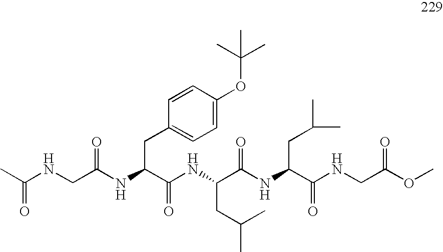

To a solution of Ac-GY(tBu)LLG-OH 228 (750 mg, 1.21 mmol) in methanol (80 mL) was added EDC (256 mg, 1.33 mmol) and DMAP (1 mg), and the mixture stirred overnight at rt. The volatiles were removed under reduced pressure, and the residue was dissolved in ethyl acetate:water (1:1, v:v, 100 mL). The organics were washed two times with saturated aqueous sodium bicarbonate, dried over magnesium sulfate, and the evaporated under reduced pressure to give 229 as a yellow oil (254 mg). This was used without purification in the next reaction. Some material was lost in the work-up procedure—unreacted CO₂H starting material was washed out in the aqueous washings.

### Example AV

### Synthesis of Linear Reference Pentapeptide A: Deprotection of 229 to Give Ac-GYLLG-OMe (230)

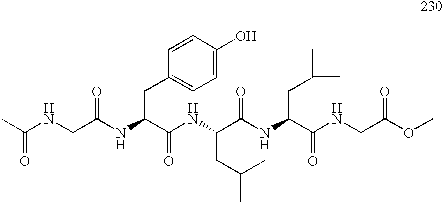

To a solution of Ac-GY(tBu)LLG-OMe 229 (58 mg, 0.09 mmol) in dichloromethane (2 mL) was added TFA (2 mL) and the solution stirred at rt for 1 h. The volatiles were removed under reduced pressure, and the residue dissolved in acetonitrile (1 mL) and water (1 mL). This solution was purified by preparative HPLC, to give the purified peptide 230 (12.6 mg) as a white solid. [M+H]⁺=578.8; Analytical HPLC (0-40% B over 40 min, mL/min, 214 nm): t_{R}=25.51 min; ¹H NMR, 600 MHz, 1:9 d₃-TFE:H₂O. Consistent with structure. The peptide was not soluble in H₂O

### Example AW

### Synthesis of Linear Reference Peptide Galanin 1-16: GWTLNSAGYLLGPHAV-NH₂ (231)

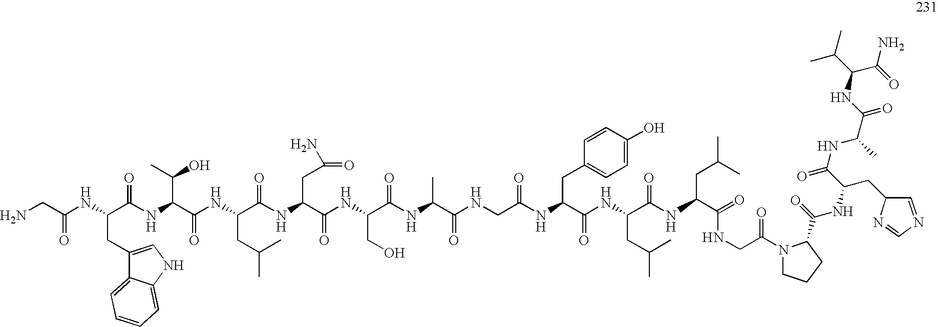

Galanin 1-16 SPPS was carried out as a test of the pentapeptide building block conditions. Thus the assembly was carried out on Rink amide MBHA resin (0.18 mmol scale) using the standard protocol, but introducing Gly5 with only 0.5 equivalents of glycine, and employing Fmoc-Tyr(2BrZ)-OH instead of the standard Fmoc-Tyr(tBu)-OH. After assembly was complete the resin was dried (still Fmoc protected) to give a yellow resin (285 mg). A sample of resin (183 mg) was cleaved following the cleavage procedure outlined in the General methods, to give a white solid (34 mg). A portion of this material (10 mg) was purified by semi-preparative HPLC to give Galanin 1-16 231 (2.3 mg). As required, more material was purified from the crude lypholised white solid. The entire synthesis was repeated to obtain more material, and now that the modified pentapeptide building block conditions were established, Gal 1-16 was assembled in a conventional manner.

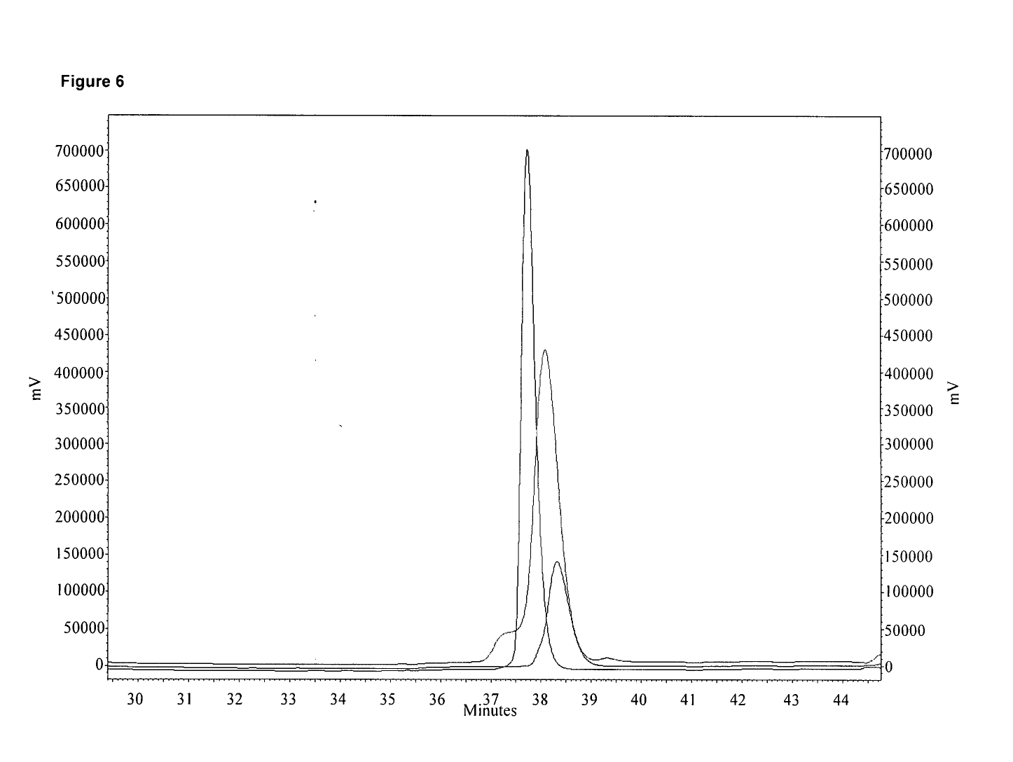

Analytical HPLC: (0-40% B over 40 mins, 1 mL/min, 214 nm): t_{R}=37.73 min (see FIG. 6).

ESI Nanospray MS: [M+2H]²⁺=827.94. Fragments were assigned and the correct peptide sequence was found.

¹H NMR (600 MHz, 9:1 H₂O: D₂O): consistent with product

### Example AX

### Synthesis of Galanin 1-16 Analog (232a) Incorporating (R)-Pentapeptide Helix Mimetic A

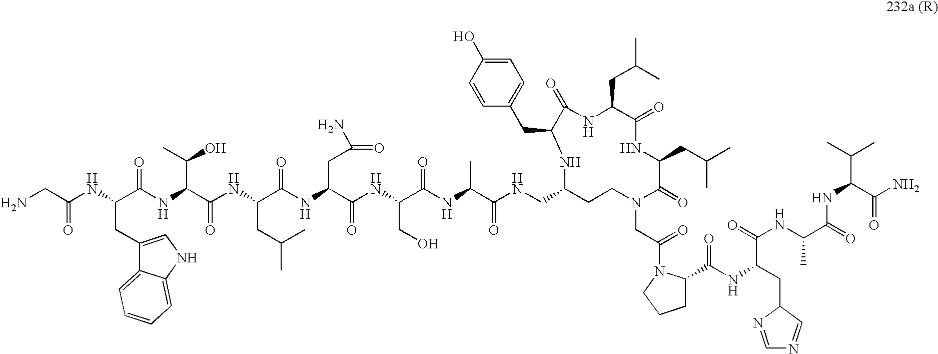

The Galanin(1-16) (R)-pentapeptide mimetic 232a was assembled according to the General SPPS procedures using Rink amide MBHA resin (255 mg), Fmoc-R pentapeptide-CO₂H building block 211a (58 mg, 0.5 equiv), and following resin cleavage gave the crude product (75 mg) as a mixture of the desired R Gal peptide and the linear peptide with the building block absent (as 0.5 equiv pentapeptide building block coupled with no acetylation). The material was purified by semi-preparative HPLC (see General Procedures) to give the product 232a as a white solid (6.7 mg).

Analytical HPLC: (0-40% B over 40 mins, 1 mL/min, 214 nm): t_{R}=38.04 min at 25° C. (see FIG. 6).

ESI Nanospray MS: [M+2H]²⁺=833.95. Fragments each side of the cyclic pentapeptide unit were assigned and the correct peptide sequence confirmed.

¹H NMR (600 MHz, 9:1 H₂O:D₂O): consistent with product

### Example AY

### Synthesis of Galanin 1-16 Analog (232b) Incorporating (S)-Pentapeptide Helix Mimetic A

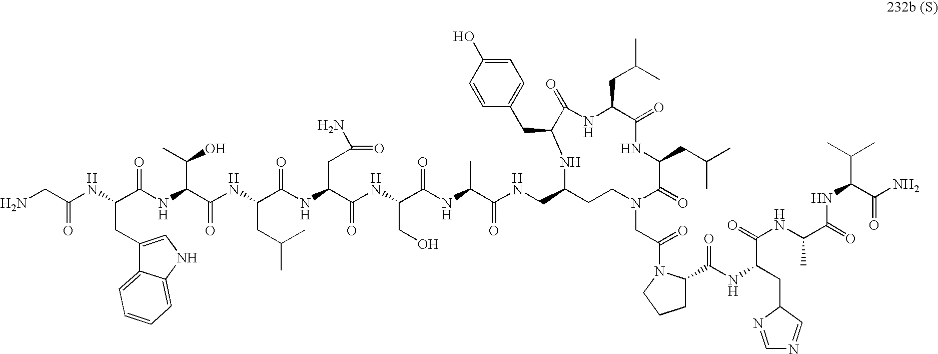

The Galanin(1-16) (S)-pentapeptide mimetic 232b was assembled according to the General SPPS procedures using Rink amide MBHA resin (383 mg), Fmoc-S pentapeptide-CO₂H building block 211b (87 mg, 0.5 equiv), and following resin cleavage gave the crude product (87 mg) as a mixture if the desired S Gal peptide and the linear peptide with the building block absent (as 0.5 equiv of pentapeptide building block coupled with no acetylation). The material was purified by semi-preparative HPLC to give the product 232b as a white solid (4.5 mg).

Analytical HPLC: (0-40% B over 40 mins, 1 mL/min, 214 nm): t_{R}=38.34 min (see FIG. 6). ESI Nanospray MS of Gal S. [M+2H]²⁺=833.95. Fragments each side of the cyclic pentapeptide unit were assigned and the correct peptide sequence confirmed. MS/MS fragments were the same as for Gal R.

¹H NMR (600 MHz, 9:1 H₂O:D₂O): consistent with product

### Example AZ

### Synthesis of Linear Reference Peptide Temporin A (native sequence): FLPLIGRVLSGIL-NH₂ (233)

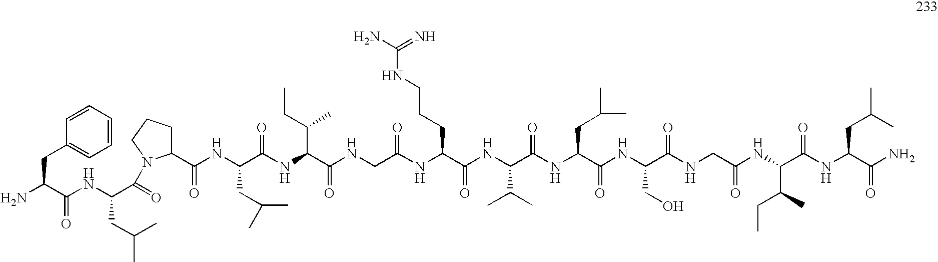

Temporin A was assembled on Rink amide MBHA resin (532 mg, 0.25 mmol scale) using standard Fmoc SPPS (see General methods) to give the N-terminal Fmoc protected peptide on-resin (730 mg). A portion of this resin (200 mg) was cleaved and gave the crude product (65 mg) as a white solid. This material was purified using preparative HPLC to give Temporin A 233 (6.4 mg) as a white solid. Analytical HPLC: (20-60% B over 30 mins, 1 mL/min, 214 nm): t_{R}=32.22 min

ESI Nanospray MS: [M+H]⁺=1396.7, [M+2H]²⁺=698.9

### Example BA

### Synthesis of Linear Reference Peptide Temporin A (V8G/I12L mutated sequence): FLPLIGRGLSGLL-NH₂ (234)

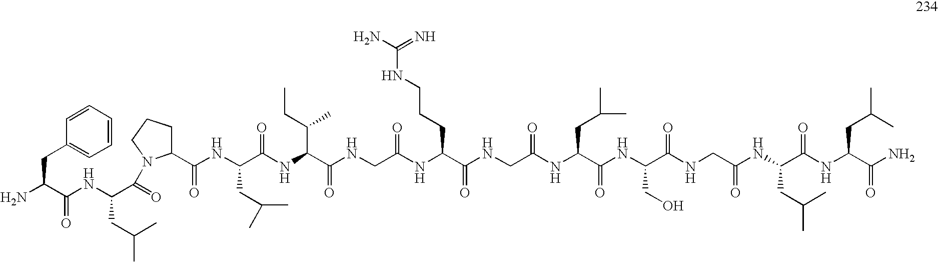

Temporin A V8G/I12L was assembled on Rink amide MBHA resin (521 mg, 0.25 mmol scale) using standard Fmoc SPPS (see General methods). All resin was Fmoc deprotected and cleaved to give the crude product (240 mg) as a white solid. A portion of this material (35 mg) was purified using preparative HPLC to give Temporin A V8G/I12L 234 (4.4 mg) as a white solid. Analytical HPLC (20-50% B over 30 mins, 1 mL/min, 214 nm): t_{R}=24.21 minESI Nanospray MS: [M+H]⁺=1355.7, [M+2H]²⁺=677.9; ¹H NMR (600 MHz, 9:1 H₂O:D₂O): consistent with product

### Example BB

### Synthesis of V8G/I12L Temporin A Analog (235a) Incorporating (R)-Pentapeptide Helix Mimetic B

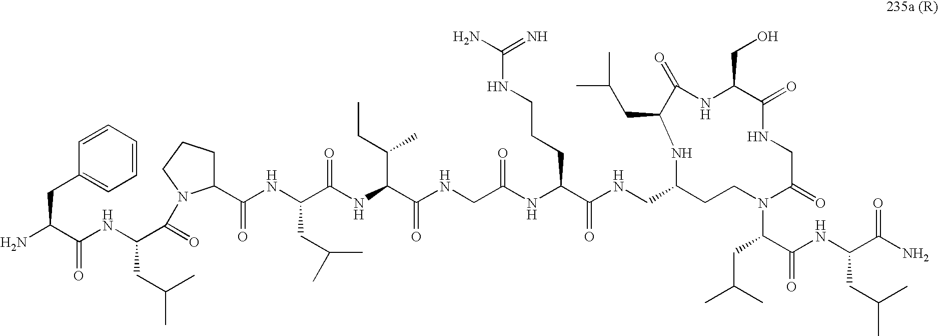

The Temporin A (R)-peptide mimetic 235a was assembled according to the General SPPS procedures using Rink amide MBHA resin (524 mg) and Fmoc-(R)-pentapeptide helix mimetic building block 227a (64 mg, 0.6 equiv). Resin cleavage gave the crude product (69 mg) as a mixture of the desired Temporin A (R)-peptide mimetic and a linear peptide with the building block absent (as only 0.5 equiv of pentapeptide building block was employed, with no capping acetylation). The benzyl ether protecting group of the serine alcohol group was still present following peptide cleavage from the resin. Initially hydrogenolysis of the crude product followed by final purification was attempted, but was not successful. Thus the crude material (69 mg) was purified by preparative HPLC to give the purified Temporin A (R)-peptide mimetic (8 mg) containing the serine benzyl ether protecting group. To a solution of this material (8 mg) in ethanol (2 mL) was added aqueous hydrochloric acid (140 μL of 8.3 μM solution, 2 equiv relative to peptide) and palladium hydroxide on carbon (9 mg). The mixture was agitated under an atmosphere of hydrogen (40 p.s.i) using a Parr Hydrogenator for 4 days. The mixture was passed through a small filter unit (0.45 μm, 25 mm), which was washed by passing through a solution of buffer A:B (1:1 v:v). The resulting solution was lypholised to give the crude serine-deprotected product (10 mg) as a white solid. This material was purified using semi-preparative HPLC to give the pure Temporin A (R)-peptide mimetic 235a (2.2 mg) as a white solid.

Analytical HPLC (20-50% B over 30 mins, 1 mL/min, 214 nm): t_{R}=21.11 min MS/MS carried out by MALDI analysis with DAN matrix: parent ion [M+H]⁺=1366.8544, main daughter 797.5439

### Example BC

### Synthesis of V8G/I12L Temporin A Analog (235b) Incorporating (S)-Pentapeptide Helix Mimetic B

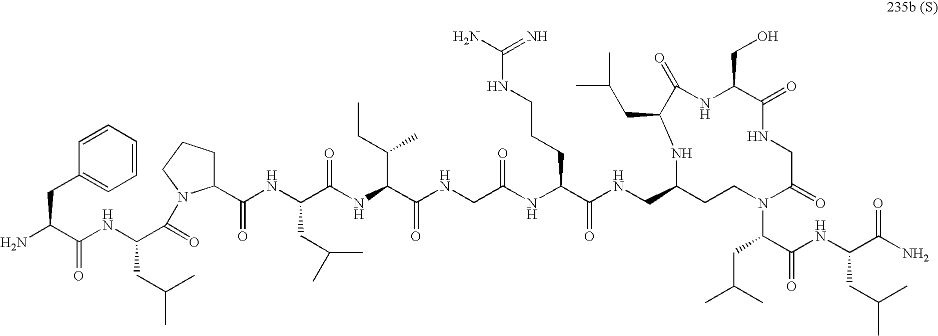

The Temporin A (S)-peptide mimetic 235b was assembled according to the General SPPS procedures using Rink amide MBHA resin (524 mg) and Fmoc-(S)-pentapeptide helix mimetic building block 227b (75 mg, 0.7 equiv). Resin cleavage gave the crude product (61 mg) as a mixture of the desired Temporin A (S)-peptide mimetic and a linear peptide with the building block absent (as only 0.7 equiv of pentapeptide building block was employed, with no capping acetylation). The benzyl ether protecting group of the serine alcohol group was still present following peptide cleavage from the resin. Initially hydrogenolysis of the crude product followed by final purification was attempted, but was not successful. A portion of this crude material (23 mg) was purified by preparative HPLC to give the purified Temporin A (S)-peptide mimetic (5 mg) containing the serine benzyl ether protecting group. To a solution of this material (5 mg) in ethanol (1.5 mL) was added aqueous hydrochloric acid (85 μL of 8.3 μM solution, 2 equiv relative to peptide) and palladium hydroxide on carbon (6 mg). The mixture was agitated under an atmosphere of hydrogen (40 p.s.i) using a Parr Hydrogenator for 4 days. The mixture was passed through a small filter unit (0.45 μm, 25 mm), which was washed by passing through a solution of buffer A:B (1:1 v:v). The resulting solution was lypholised to give the crude serine-deprotected product (11 mg) as a white solid. This material was purified using semi-preparative HPLC to give the pure Temporin A (S)-peptide mimetic 235b (2.1 mg) as a white solid.

Analytical HPLC (20-50% B over 30 mins, 1 mL/min, 214 nm): t_{R}=18.68 min

MS/MS carried out by MALDI analysis with DAN matrix: parent ion [M+H]⁺=1366.8433, main daughter 797.5433

¹H NMR (600 MHz, 9:1 H₂O:D₂O): consistent with product

### Example BD

### CD spectra: Galanin Linear Reference Pentapeptide A (230), (R)-Pentapeptide Helix Mimetic A (213a) and (S)-Pentapeptide Helix Mimetic A (213b)

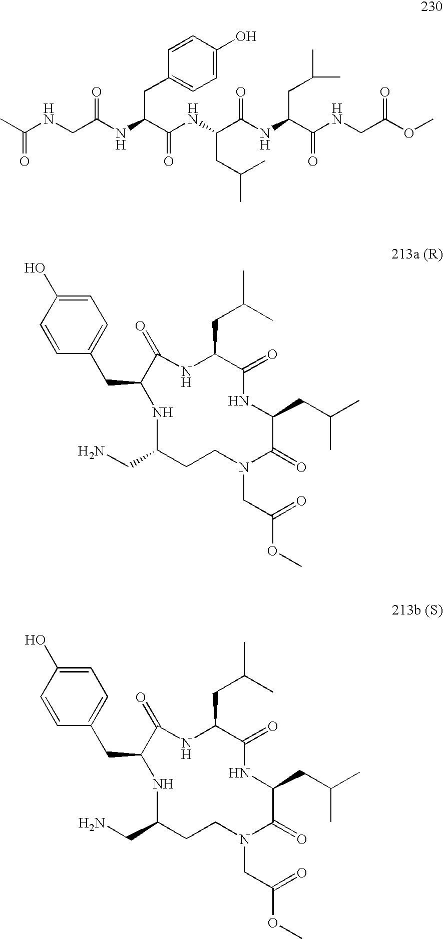

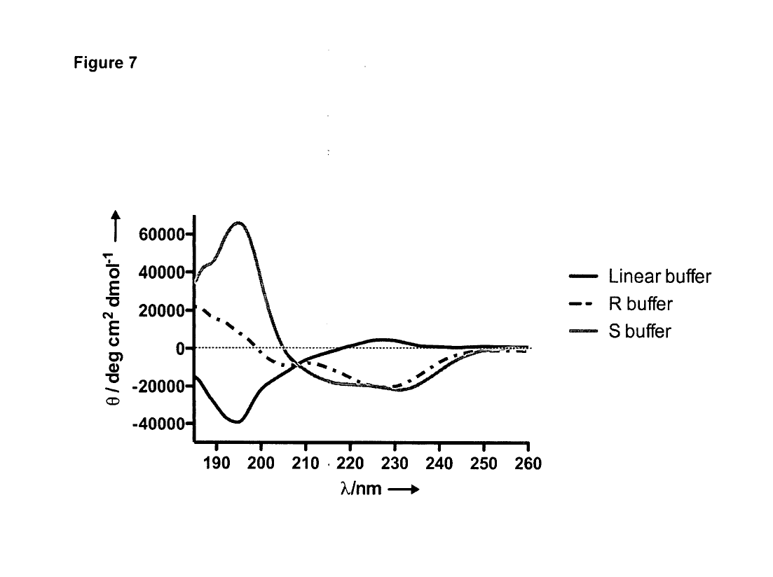

CD spectroscopy (as outlined in Example K) was conducted on the Galanin reference linear pentapeptide A (230) and the pentapeptide helix mimetics 213a and 213b in 10 mM phosphate buffer. CD spectra of both 213a and 213b show a double minima and positive absorbance at 195 nm characteristic of an α-helical structure whereas the linear analogue Ac-GYLLG-Me (230) showed random coil behaviour (see FIG. 7). In particular the CD spectrum of 213b shows a negative 208 nm and large positive 195 nm absorbance typical of an α-helical conformation, arising from the exciton splitting of the π→π transition that occurs when amides are aligned in an α-helix conformation.

### Example BE

### CD spectra: Galanin(1-16) Linear Reference Peptide (231) and Galanin(1-16) Analogs Incorporating (R)-Helix Pentapeptide Mimetic A (232a) and (S)-Helix Pentapeptide Mimetic A (232b)

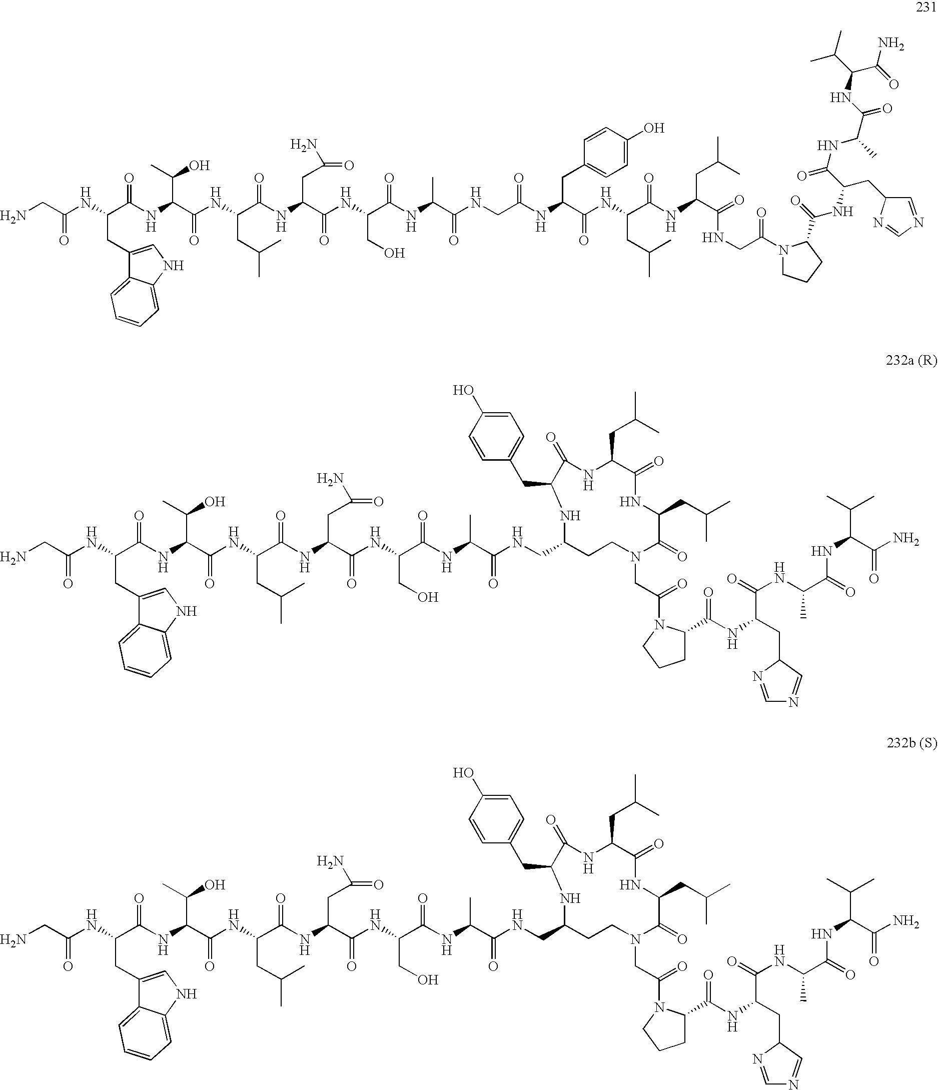

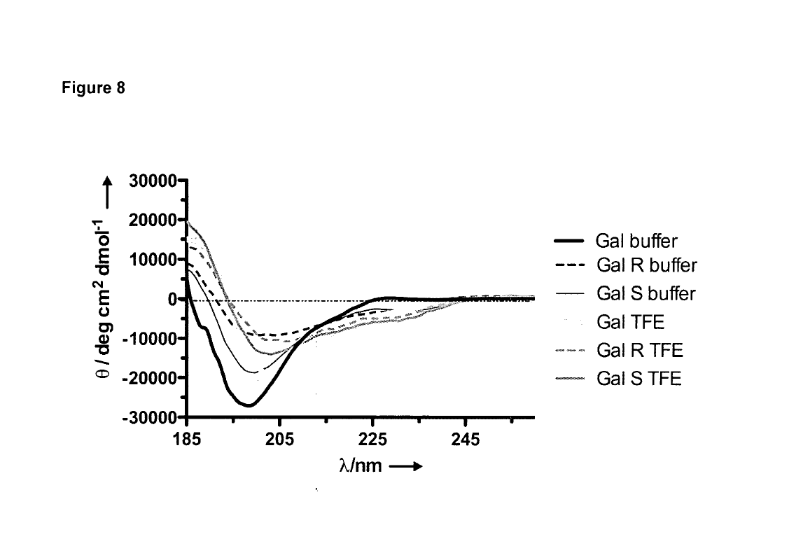

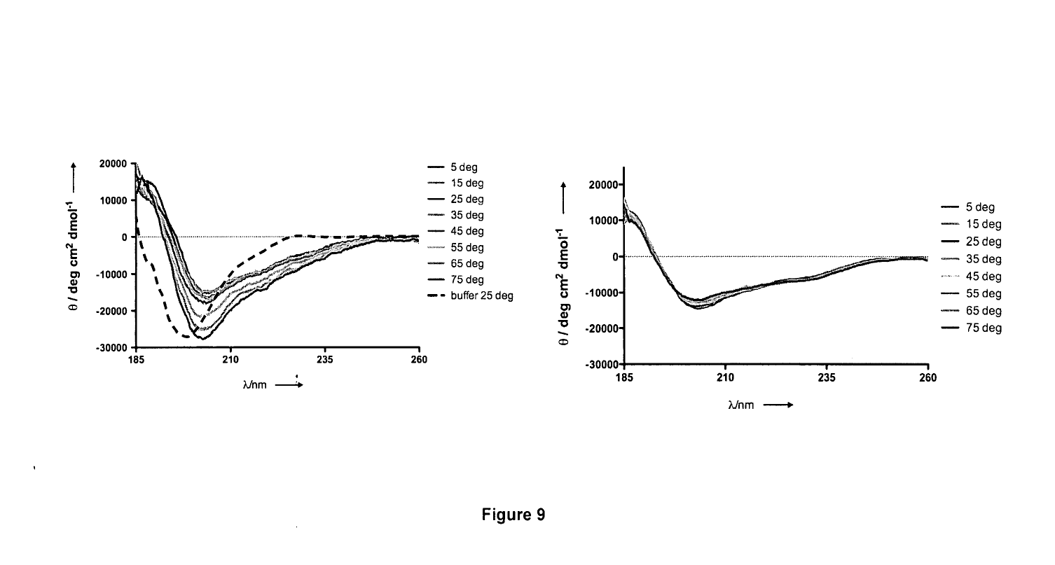

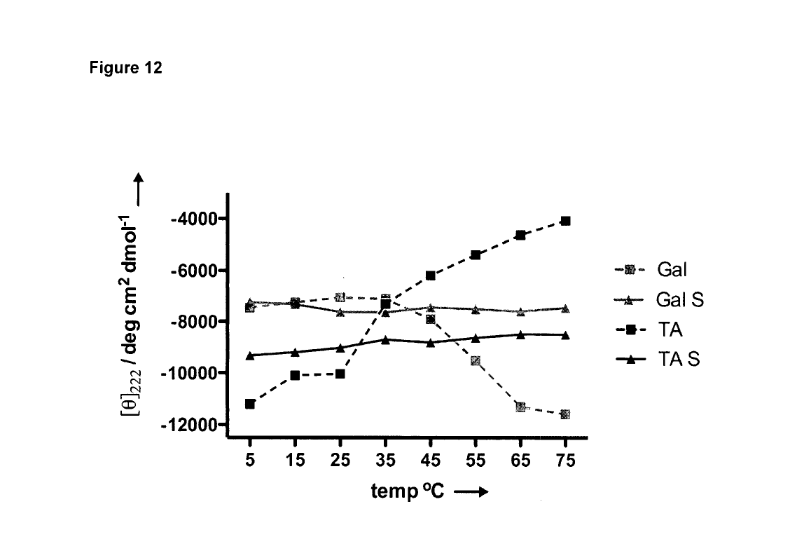

CD spectroscopy (as outlined in Example K) was conducted on the Galanin(1-16) reference linear peptide 231 and the Galanin(1-16) helix mimetics 232a and 232b in 10 mM phosphate buffer (pH 7.4) and in 10 mM phosphate buffer with 30% TFE added (see FIG. 8). TFE is known to enhance or stabilise peptide secondary structure, in particular canonical α-helix conformers
The spectrum of Gal(1-16) 231 in buffer shows a deep minimum at 195 nm that is typical of a random coil, and with added TFE shows a slight increase in helical structure. The CD spectra of 11a and 11b show a more helical structure than linear 10 in both buffer and buffer with TFE. Comparison of the diastereoisomers reveals subtle differences, with the S isomer 11b displaying more typical α-helical behaviour (negative 205, 222 nm) with a larger positive 190 nm transition, whereas the R isomer 11a reveals a structure that is barely altered by the addition of TFE. The CD spectra of 231, 232a, and 232b were also recorded in buffer/sodium dodecyl sulfate (SDS) solutions, with results comparable to those obtained in 30% TFE solutions.
Further CD experiments examined the thermal denaturation secondary structure stability of Gal(1-16) 231 and (S)-Gal(1-16) helix mimetic 232b were investigated by thermal denaturation experiments (FIG. 9). Variable temperature CD spectra of Gal(1-16) 231 show decreased peptide secondary structure with increased temperature (minima deepens and moves to the left). Remarkably different are the variable temperature CD spectra of H-bond mimetic S isomer 232b, which show little difference from 5-75° C. Thus 232b shows excellent structural stability at elevated temperatures. The differences in stability of 231 abd 232b are especially notable when the molar ellipticity at 222 nM is plotted (FIG. 12). The decreased 222 nm absorbance of 231 with increasing temperature is not representative of increased helicity as the 195 nm minima dramatically drops with increased temperature indicating loss of π→π* exiton splitting hence helical structure.

### Example BF

### CD spectra: Temporin A V8G/I12L Linear Reference Peptide (234) and Temporin A Analogs Incorporating (R)-Helix Pentapeptide Mimetic B (235a) and (S)-Helix Pentapeptide Mimetic B (235b)

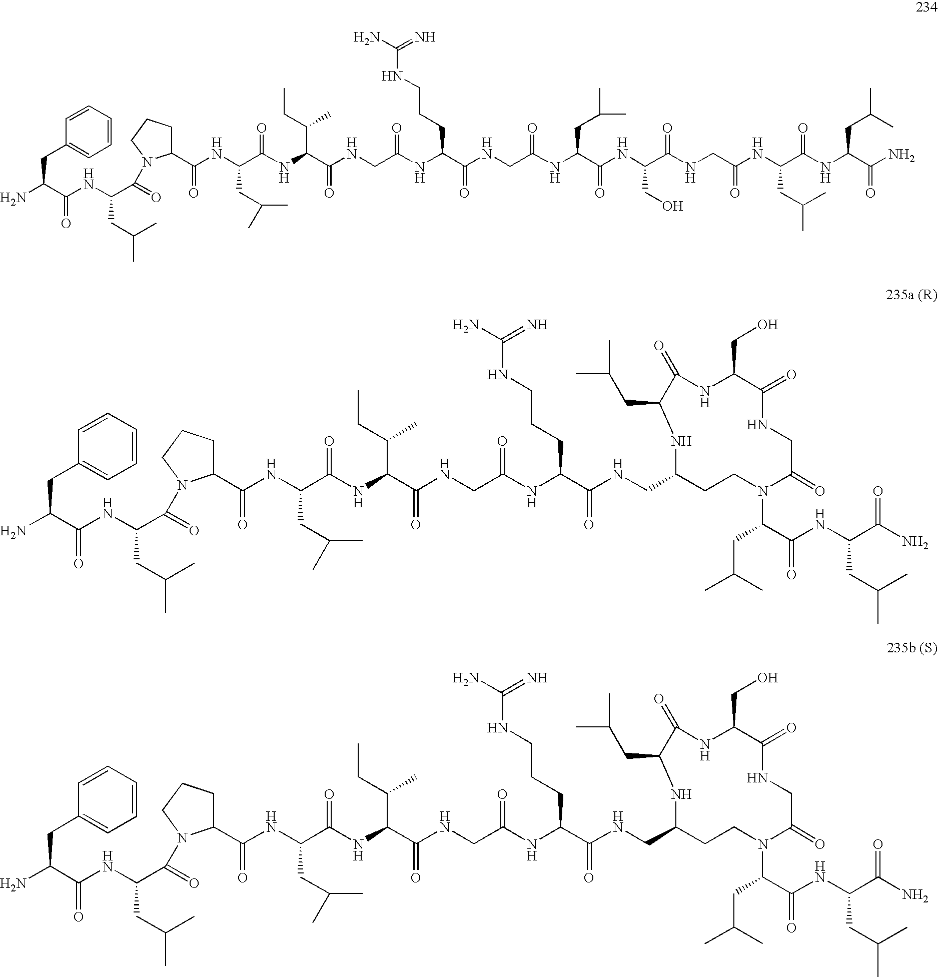

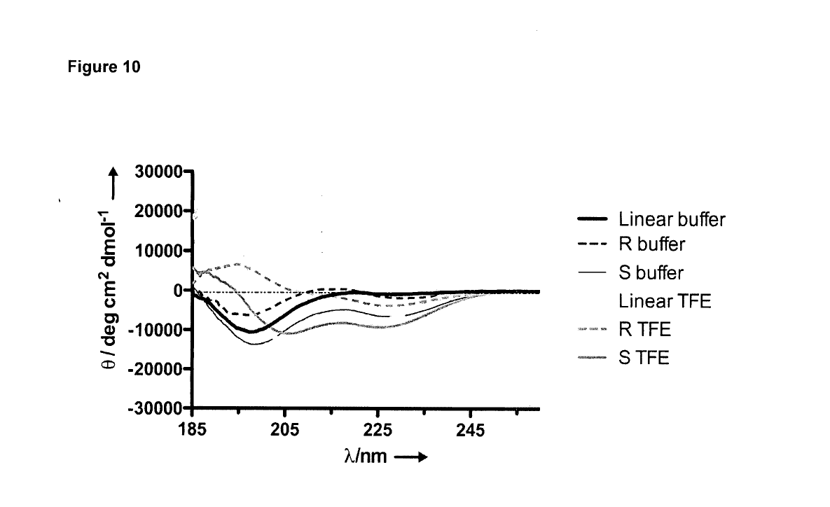

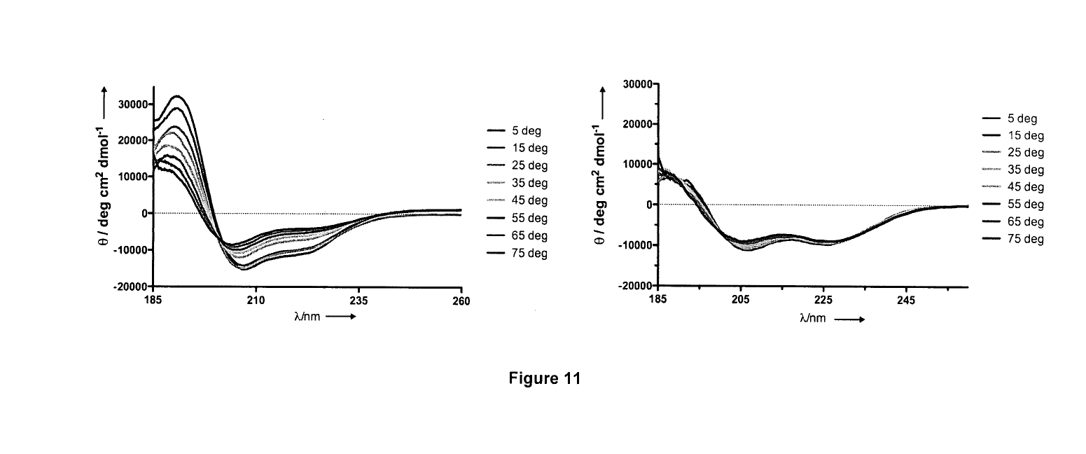

CD spectroscopy (as outlined in Example K) was conducted on the Temporin A V8G/I12L Linear Reference Peptide 234, Temporin A Analog Incorporating (R)-Helix Pentapeptide Mimetic B 235a and Temporin A Analog Incorporating (S)-Helix Pentapeptide Mimetic B 235b in 10 mM phosphate buffer (pH 7.4) and in 10 mM phosphate buffer with 30% TFE added (see FIG. 10).
The CD spectra of Temporin A linear reference peptide 234 and the Temporin A analog incorporating (R)-helix pentapeptide mimetic B 235a recorded in buffer show a relatively unordered structure, while some helicity was observed for Temporin A analog incorporating (S)-helix pentapeptide mimetic B 235b (FIG. 10). Upon addition of TFE the CD spectrum of 234 shows a large 190 nm maximum and double minima at approximately 208 nm and 222 nm, characteristic of α-helical structure. Addition of TFE to 235a significantly changed the peptide conformation, but not into an α-helical structure. The CD spectrum of 235b with added TFE shows a well-defined double minima indicating a helix, although this is not a typical α-helix motif as the 190 nm absorbance is moderate and the second minima is at 226 nm rather than 222 nm.
The secondary structure stability of linear Temporin A analog 234 and Temporin A helix mimetic 235b was investigated by thermal denaturation experiments (FIGS. 11 and 12). Linear Temporin A analog 234 shows an increase in 222 nm absorbance as temperature increases, indicative of the helix unwinding. In comparison Temporin A helix mimetic 235b shows a remarkable thermal stability, with the 222 nm absorbance almost constant throughout the 70° C. temperature range.

### Example BH

### Galanin Peptides Radioligand Binding Assay

Galanin(1-16) Linear Reference Peptide 231, Galanin(1-16) Analog Incorporating (R)-Helix Pentapeptide Mimetic A 232a and Galanin(1-16) Analog Incorporating (S)-Helix Pentapeptide Mimetic A 232b were tested for binding affinity to the Galanin-1 and Galanin-2 receptors using a radioligand binding competitive assay (Heuillet E, Bouaiche Z, Menager J, Dugay P, Munoz N, Dubois H, Amiranoff B, Crespo A, Lavayre J, Blanchard J C, Doble A; Eur J. Pharmacol., 1994, 269, 139-147. Bloomquist B T, Beauchamp M R, Zhelnin L, Brown S E, Biochem Biophys Res Comm., 1998, 243, 474-479.) Human recombinant HEK-293 cells were used as the source of Galanin GAL1, and samples tested against competitive binding with 0.012 nM ¹²⁵I Galanin (porcine). DMSO was used as the vehicle, and the incubation buffer consisted of 10 mM HEPES-NaOH, pH 7.4, 1 mM EDTA, 0.5% BSA, 0.01% Bacitracin. Human recombinant CHO-K1 cells were used as the source of Galanin GAL2, and samples tested against competitive binding with 0.02 nM ¹²⁵I Galanin (human). Native Gal 1-16 231 synthesised in this study gave comparable results to literature (see Table 2).

TABLE 2
Inhibition of ¹²⁵I Galanin Binding to GAL1 and GAL2 Receptors

| | GAL1 | | GAL2 | |
|---|---|---|---|---|
| | % inhib at | % inhib at | % inhib at | % inhib at |
| | 100 nM | 10 nM | 1 1000 nM | 100 nM |
| Gal (1-16) | 96 | 85 | 101 | 92 |
| 231 | | | | |
| Gal R 232a | 37 | 8 | 36 | 6 |
| Gal S 232b | 18 | 2 | 6 | 2 |

### Example BI

### Galanin Peptides Rat Plasma Stability

Rat plasma was obtained freshly from male Sprague Dawley rats. The stock solutions for the samples Galanin(1-16) Linear Reference Peptide 231, Galanin(1-16) Analog Incorporating (R)-Helix Pentapeptide Mimetic A 232a and Galanin(1-16) Analog Incorporating (S)-Helix Pentapeptide Mimetic A 232b were prepared in 1:1 v:v acetonitrile/water at a concentration of 1 mg/mL. This stock solution was further diluted using 1:1 v:v acetonitrile/water to give a spiking solution with a nominal concentration of 50 μg/mL. For each compound, an aliquot (1485 μL) of rat plasma (maintained at 37° C.) was spiked with 15 μL of the spiking stock to give a nominal plasma concentration of 500 ng/mL. Samples were incubated at 4° C. and 37° C. and duplicate samples were taken at various time points over 140 min. Samples were quenched by snap freezing in dry-ice. The concentration of the parent compound (Gal 231, Gal R 232a, Gal S 232b) was determined by LC/MS on a Micromass Quattro Premier triple-quadrupole instrument. The percentage of the parent compound remaining at each time point was calculated relative to the concentrations observed in the spiking control samples.

TABLE 3
Rat Plasma Stability of Galanin Peptides

| | Time | % Compound | % Compound |
|---|---|---|---|
| | (min) | remaining at 4° C. | remaining at 37° C. |
| Gal (1-16) 231 | 2 | — | 30.2 |
| | 30 | — | BL |
| | 240 | 6.2 | ND |
| Gal R 232a | 2 | — | 50.0 |
| | 30 | — | BL |
| | 240 | 31.3 | ND |
| Gal S 232b | 2 | — | 44.8 |
| | 30 | — | BL |
| | 240 | 31.3 | ND |

BL—below lower limit of quantitation. ND = not detected

Both the Gal(1-16) 231 and Gal(1-16) mimetics 232a and 232b degraded rapidly in rat plasma when incubated at 37° C., with concentrations below the limit of quantitation within 30 min of spiking. At 4° C., the rate of degradation was slower for all three compounds, and the percent remaining at the end of 240 min incubation was significantly higher for 11a and 11b than for 10 (see Table 3), indicating that the helix mimetic template provides protection from proteolysis when incorporated into a peptide.

### INDUSTRIAL UTILITY

As demonstrated in Examples BD to BI, compounds of the invention of Type IIIa show enhanced helical secondary structure and improved biological properties such as stabilisation against proteolytic degradation.

### REFERENCES

Abdel-Magid, A. F., K. G. Carson, et al. (1996). Journal of Organic Chemistry 61: 3849-3862.
Alkorta, I.; M. L. Suarez, et al. (1996). J. Mol. Model. 1: 16-25.
Arrhenius, T., R. A. Lerner, et al. (1987). The chemical synthesis of structured peptides using covalent hydrogen-bond mimics. In Protein Structure, Folding and Design 2 Ed. D. Oxender. Alan R. Liss, Inc. 453-465.
Ball, J. B. and P. F. Alewood (1990). Journal of Molecular Recognition 3(2): 55-64.
Ball, J. B., R. A. Hughes, et al. (1993). Tetrahedron 49(17): 3467-3478.
Basile, T., A. Bocoum, et al. (1994). Journal of Organic Chemistry 59: 7766-7773.
Bernotas, R. C. and R. V. Cube (1991). Tetrahedron Letters 32(2): 161-164.
Bocoum, A., C. Boga, et al. (1991). Tetrahedron Letters 32: 1367-1370.
Bodansky, M. and A. Bodansky (1984). The Practice of Peptide Synthesis. Berlin-Heidelberg, Springer-Verlag.
Borch, R. F., M. D. Bernstein, et al. (1971). J. Am. Chem. Soc. 93: 2897-2904.
Boutin, R. H. and H. Rapoport (1986). Journal of Organic Chemistry 51: 5320-5327.
Brown, H. C. (1975). Organic Syntheses Via Boranes. New York, Wiley.
Brown, H. C. and e. al. (1986). Tetrahedron 42: 5515.
Brown, H. C. and K. S. Bhat (1986). J. Am. Chem. Soc. 108: 293.
Brown, H. C. and P. K. Jadhav (1983). J. Am. Chem. Soc. 105: 2092-2093.
Brown, H. C. and P. K. Jadhav (1984). Journal of Organic Chemistry 49: 4089.
Brown, H. C. and Krishnamurthy (1979). Tetrahedron 35: 567-607.
Brown, H. C., S. U. Kulkarni, et al. (1980). Synthesis: 151.
Brown, H. C., R. S. Randad, et al. (1990). J. Am. Chem. Soc. 112: 2389.
Callahan, J. F., J. W. Bean, et al. (1992). J MedChem 35: 3970-3972.
Carlock, J. T. and M. P. Mack (1978). Tetrahedron Letters 52: 5153-5156.
Carpino, L. A., A. El-Faham, et al. (1994). Tetrahedron Letters 35: 2279-2280.
Carpino, L. A., D. Sadat-Aalaee, et al. (1990). J. Am. Chem. Soc. 112: 9651-9652.
Chalmers, D. K. and G. R. Marshall (1995). J. Am. Chem. Soc. 117(22): 5927-37.
Chen, S., R. A. Chrusciel, et al. (1992). PNAS 89(Biochemistry): 5872-5876.
Cupps, T. L., R. H. Boutin, et al. (1985). Journal of Organic Chemistry 50: 3972-3979.
Ehrlich, A., S. Rothemund, et al. (1993). Tetrahedron Letters 34: 4781-4784.
Farmer, P. S, and E. J. Ariëns (1982). Topics in Peptide Science: 362-365.
Fehrentz, J.-A. and B. Castro (1983). SYNTHESIS: 676-678.
Frigerio, M. and M. Sangostino (1994). Tetrahedron Letters 35: 8019-8022.
Früchtel, J. S, and G. Jung (1996). Angew. Chem. Int. Ed. Engl. 35: 17-42.
Galéotti, N., C. Montagne, et al. (1992). Tetrahedron Letters 33(20): 2807-2810.
Gallop, M. A., R. W. Barrett, et al., (1994). Journal of Medicinal Chemistry 37: 1233-1251.
Gardner, B., H. Nakanishi, et al. (1993). Tetrahedron 49(17): 3433-3448.
Giannis, A. and T. Kolter (1993). Angew. Chem., Int. Ed. Enql. 32: 1244-1267.
Gordon, E. M., R. W. Barrett, et al. (1994). Journal of Medicinal Chemistry 37: 1385-1401.
Greene, T. W. and P. G. Wuts (1991). Protective Groups. New York, John Wiley & Sons.
Gribble and Nutatits (1985). Org. Prep. Proc. Int. 17: 317.
Griffith, W. P. and S. V. Ley (1990). Aldrichimica Acta 23: 13-19.
Guilbourdenche, C., R. Lazaro, et al. (1994). Bull. Soc. Chim. Belg. 103(1): 1-8.
Henry, J. R., L. R. Marcin, et al. (1989). Tetrahedron Letters 30(42): 5709-5712.
Hirschmann, R., K. C. Nicolau, et al. (1993). J. Am. Chem. Soc. 115: 12550-12568.
Hirschmann, R., K. C. Nicolau, et al. (1992). J. Am. Chem. Soc. 114: 9217-9218.
Hirschmann, R., W. Yao, et al. (1996). Tetrahedron Letters 37: 5637-5640.
Hölzemann, G. (1991). Kontakte (Darmstadt): 3-12.
Hölzemann, G. (1991). Kontakte (Darmstadt): 55-63.
Hudlicky, M. (1990). Oxidations in Organic Chemistry. Washington, American Chemical Society.
Huffman, W. F., J. F. Callahan, et al. (1989). Mimics of Secondary Structural Elements of Peptides and Proteins. Synthetic Ppetides: Approaches to Biological Problems. Alan R. Liss, Inc. 257-266.
Huffman, W. F., J. F. Callahan, et al. (1988). Reverse turn mimics. Peptides: Chemistry and Biology, Proceedings of the Tenth American Peptide Symposium Ed.
G. R. Marshall. Leiden, The Netherlands, ESCOM. 105-108.
Hughes, D. L. (1992). Organic reactions 42: 335-656.
Humphries, M. J., P. M. Doyle, et al. (1994). Exp. Opin. Ther. Patents 4(3): 227-235.
Hutchins, R. O. and N. R. Natale (1979). Org. Prep. Proc. Int. 11: 203-241.
Jurczak, J. and A. Golebiowski (1989). Chem. Rev. 89: 149-164.
Kahn, M. (1993). SYNLETT: 821-826.
Kahn, M. (1996). Library of conformationally constrained reverse-turn peptides. 64 pp. PCT Int. Appl., Molecumetics, Ltd., USA.
Kelly, J. W., N. L. Eskew, et al. (1986). Journal of Organic Chemistry 51: 95-97.
Kessler, H., B. Diefenbach, et al. (1995). Letters in Peptide Science 2: 155-160.
Knapp, S., J. J. Hale, et al. (1992). Journal of Organic Chemistry 57: 6239-6256.
Koskinen, A. M. P. and H. Rapoport (1989). Journal of Organic Chemistry 54: 1859-1866.
Kramer, G. W. and H. C. Brown (1974). Journal of Organometallic Chemistry 73: 1-15.
Kramer, G. W. and H. C. Brown (1977). Journal of Organometallic Chemistry 132: 9-27.
Krstenansky, J. L., R. L. Baranowski, et al. (1982). Biochem. Biophys. Res. Commun. 109:1368-1374.
Kuntz, I. D. (1972). J. Am. Chem. Soc. 94: 4009-4012.
Lewis, P. N., F. A. Momany, et al. (1973). Biochim. Biophys. Acta 303: 211-229.
Ma, X., G. Passaro, et al. (1995). Protein and Peptide Letters: 347-350.
Meier, H. and K. Zeller (1975). Angew. Chem.,Int. Ed. Engi. 14: 32-43.
Milner-White, E. J. (1988). Journal of Molecular Biology 204: 777-782.
Nahm, S. and S. M. Weinreb (1981). Tetrahedron Letters 22(39): 3815-3818.
Nakanishi, H. and M. Kahn (1996). Design of Peptidomimetics. The Practice of Medicinal Chemistry. Academic Press Limited. Ch. 27, 571-590.
Newlander, K. A., J. F. Callahan, et al. (1993). JMedChem 36: 2321-2331.
Olson, G. L., D. R. Bolin, et al. (1993). Journal of Medicinal Chemistry 36: 3039-3049.
Pelter, Smith, et al. (1988). Borane Reagents. New York, Academic Press.
Pfister, J. R. (1984). Synthesis: 969-970.
Qabar, M., J. Urban, et al. (1996). Letters in Peptide Science 3: 25-30.
Racherla, U.S., Y. Liao, et al. (1992). Journal of Organic Chemistry 57: 6614-6617.
Ray, R. and D. S. Matteson (1980). Tetrahedron Letters 21: 449-450.
Richardson, J. S. (1981). Adv. Prot. Chem. 34: 167-339.
Robinson, P. L., C. N. Barry, et al. (1983). Journal of Organic Chemistry 48: 5396-5398.
Rose, G. D., L. M. Gierasch, et al. (1985). Advan. Protein Chem. 37: 1-109.
Sardina, F. J. and H. Rapoport (1996). Chem. Rev. 96: 1825-1872.
Soderquist, J. A. and M. R. Najafi (1986). Journal of Organic Chemistry 51: 1330.
Szeja, W. (1985). Synthesis: 983.
Thompson, L. A. and J. A. Ellman (1996). Chem. Rev. 96: 555-600.
Valle, G., M. Crisma, et al. (1989). International Journal of Peptide and Protein Research 33: 181-190.
VanRheenen, V., R. C. Kelly, et al. (1976). Tetrahedron Letters 23: 1973-1976.
Virgilio, A. A. and J. A. Ellman (1994). J. Am. Chem. Soc. 116(25): 11580-1.
Virgilio, A. A., S. C. Schürer, et al. (1996). Tett. Lett. 37: 6961-6964.
Wenschuh, H., M. Beyermann, et al. (1994). Journal of Organic Chemistry 59: 3275-3280.
Wilmont, C. M. and J. M. Thornton (1990). Protein Engineering 3: 479-493.
Wolf, J. and H. Rapoport (1989). Journal of Organic Chemistry 54: 3164-3173.
Yamamoto, Y. and N. Asao (1993). Chem. Rev. 93: 2207-2293.

1. A general mimetic of the structure 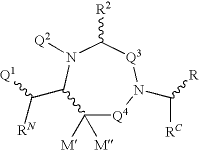
wherein:—
˜˜˜indicates a bond at a chiral centre of the structure which centre may be in the R or S configuration or a mixture thereof;
R and R² are each independently an amino acid side chain group which may be the same or different;
M′ and M″ may be the same or different and are selected from the group consisting of hydrogen, C₁-C₄ alkyl, chloro and C₁-C₄ alkoxy;
R^{N} is —N(Z′)Pg^{N} where Z′ is selected from the group consisting of hydrogen, methyl and part of a cyclic amino acid sidechain joined to Q¹, and wherein Pg^{N} is selected from the group consisting of a protecting group for an amine, a cleavable linker to a solid support, the solid support, hydrogen, R, C(O)R and part of the remaining N-terminal portion of the mimetic;
R^{C} is selected from the group consisting of a carboxy terminal part of the mimetic, hydrogen, R, and CH₂R;
Q¹=R¹ which has the same definition as R and R² above and Q²=Z where Z is selected from the group consisting of hydrogen, methyl, ethyl, formyl and acetyl, —CH₂R, and C(O)R or alternatively Z is part of a cyclic amino acid sidechain group joined to R²; or Q¹ and Q² taken together represent a cyclic group;
Q³ is selected from the group consisting of —C(O)NHCH(R)Y—, and —C(O)ENHCH(R)Y—, —wherein Y is C(O) and E is (AA)ₙ where n is 1 and M is an amino acid residue; and
Q⁴ is CH(M′),

2. A mimetic according to claim 1 wherein Q³ is —C(O)NHCH(R)Y—.

3. A mimetic according to claim 1 wherein Q³ is —C(O)ENHCH(R)Y—

4. A mimetic according to claim 3 wherein E is a group of the formula: 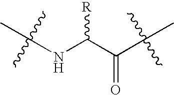
wherein R is an amino acid side chain group.

5. A mimetic according to claim 1 wherein Q² is H or Me.

6. A mimetic according to claim 1 wherein M′ and M″ are both H.

7. A mimetic according to claim 1 wherein R^{C} is C(O)Pg^{C} or CO₂H where Pg^{C} is a protecting group for carboxylic acid, a cleavable linker to a solid support, the solid support, hydroxy, NHR or the remaining C-terminal portion of the mimetic.

8. A mimetic as claimed in claim 7 wherein Pg^{C} is a protecting group for carboxylic acid selected from the group consisting of alkoxy, benzyloxy, allyloxy, fluorenylmethyloxy, and amines forming easily removable amides.

9. A mimetic as claimed in claim 8 wherein Pg^{C} is methoxy, ethoxy or butoxy.

10. A mimetic as claimed in claim 7 wherein Pg^{C} is the remaining C-terminal portion of the mimetic, wherein the remaining C-terminal portion of the mimetic is an amine, amino acid or peptide.

11. A mimetic as claimed in claim 1 wherein Q¹ is H

12. A mimetic as claimed in claim 1 wherein Z′=H

13. A mimetic as claimed in claim 1 wherein Pg^{N} is H, the remaining N-terminal portion of the mimetic or a protecting group for an amine.

14. A mimetic as claimed in claim 13 wherein the protecting group for an amine is selected from a group consisting of Boc, Fmoc, Cbz, Alloc, and trityl.

15. A mimetic as claimed in claim 13 wherein Pg^{N} is the remaining N-terminal portion of the mimetic, wherein the remaining N-terminal portion of the mimetic is an amino acid or peptide.

16. A mimetic as claimed in claim 1 wherein each R, R¹ and R² is independently selected from the group consisting of alkyl, optionally substituted alkyl, aryl, or optionally substituted aryl.
